SEQUENCE LISTING (22 sequences)

SEQ ID NO: 1 - DNA, 20 bp, Artificial Sequence
  Description of Artificial Sequence  Synthetic Primer
cgcttgagcgcagacatctt

SEQ ID NO: 2 - DNA, 20 bp, Artificial Sequence
  Description of Artificial Sequence  Synthetic Primer
ctgatatcgtcgatatcgat

SEQ ID NO: 3 - DNA, 22 bp, Artificial Sequence
  Description of Artificial Sequence  Synthetic Primer
ctccttcaaagaggcaaaaata

SEQ ID NO: 4 - DNA, 20 bp, Artificial Sequence
  Description of Artificial Sequence  Synthetic Primer
cacttcctccaggatgttgt

SEQ ID NO: 5 - DNA, 17 bp, Artificial Sequence
  Description of Artificial Sequence  Synthetic Primer
tggtacaagctagaggc

SEQ ID NO: 6 - DNA, 16 bp, Artificial Sequence
  Description of Artificial Sequence  Synthetic Primer
gcatggcacattgaac

SEQ ID NO: 7 - DNA, 23 bp, Artificial Sequence
  Description of Artificial Sequence  Synthetic Primer
cgggatcgtggaagggctaatga

SEQ ID NO: 8 - DNA, 25 bp, Artificial Sequence
  Description of Artificial Sequence  Synthetic Primer
cttcacgaagttgtcattgagggca

SEQ ID NO: 9 - DNA, 22 bp, Artificial Sequence
  Description of Artificial Sequence  Synthetic Primer
ccgagatgtggaactggcagag

SEQ ID NO: 10 - DNA, 22 bp, Artificial Sequence
  Description of Artificial Sequence  Synthetic Primer
cggagaggaggctgactttctc

SEQ ID NO: 11 - DNA, 21 bp, Artificial Sequence
  Description of Artificial Sequence  Synthetic Primer
ccacctcaatggacagaacat

SEQ ID NO: 12 - DNA, 22 bp, Artificial Sequence
  Description of Artificial Sequence  Synthetic Primer
ccatctttaggaagacacgggt

SEQ ID NO: 13 - DNA, 24 bp, Artificial Sequence
  Description of Artificial Sequence  Synthetic Primer
gaaagaggaacaactactgctggt

SEQ ID NO: 14 - DNA, 23 bp, Artificial Sequence
  Description of Artificial Sequence  Synthetic Primer
gaactgagggtacatgctggagc

SEQ ID NO: 15 - DNA, 24 bp, Artificial Sequence
  Description of Artificial Sequence  Synthetic Primer
cctacccccaatgtatccgttgtg

SEQ ID NO: 16 - DNA, 24 bp, Artificial Sequence
  Description of Artificial Sequence  Synthetic Primer
ggaggaatgggagttgctgttgaa

SEQ ID NO: 17 - DNA, 24 bp, Artificial Sequence
  Description of Artificial Sequence  Synthetic Primer
cttctgtctactgaacttcggggt

SEQ ID NO: 18 - DNA, 21 bp, Artificial Sequence
  Description of Artificial Sequence  Synthetic Primer
tggaactgatgagagggagcc

SEQ ID NO: 19 - DNA, 24 bp, Artificial Sequence
  Description of Artificial Sequence  Synthetic Primer
gagagacaagcaacgacaaaatcc

SEQ ID NO: 20 - DNA, 24 bp, Artificial Sequence
  Description of Artificial Sequence  Synthetic Primer
ttcccatcttcttctttgggtatt

SEQ ID NO: 21 - DNA, 19 bp, Artificial Sequence
  Description of Artificial Sequence  Synthetic Primer
ccaagccagacctcacagc

SEQ ID NO: 22 - DNA, 24 bp, Artificial Sequence
  Description of Artificial Sequence  Synthetic Primer
ccgataccactcttctgtttcttg

(19) United States
(12) Patent Application Publication
(10) Pub. No.: US 2009/0111812 A1
(43) Pub. Date: Apr. 30, 2009

(54) METHODS FOR TREATING INFLAMMATORY DISORDERS

(75) Inventors: Inderjit Singh, Mount Pleasant, SC (US); Avtar Singh, Mount Pleasant, SC (US)

Correspondence Address: FULBRIGHT & JAWORSKI L.L.P., 600 CONGRESS AVE., SUITE 2400, AUSTIN, TX, 78701, US

(73) Assignee: MUSC Foundation for Research Development, Charleston, SC (US)

(21) Appl. No.: 11/570,635

(22) PCT Filed: Jun. 13, 2005
(86) PCT No.: PCT/US2005/020664

§ 371 Date: Oct. 29, 2008

Related U.S. Application Data
(60) Provisional application No. 60/579,548, filed on Jun. 14, 2004.

(51) Int. Cl. A61K 31/5375; A61K 31/44; A61P 3/10

(52) U.S. Cl. 514/237.8; 514/348

(57) ABSTRACT

The present invention relates generally to the fields of molecular biology. More particularly, it concerns materials and methods for the treatment of nitric oxide and cytokind mediated disorders. In a preferred embodiment, PDMP may be used to inhibit the expression of iNOS and pro-inflammatory cytokines such as TNFα and IL1β.

The government owns rights in the present invention pursuant to grant number NS-22576, NS-34741, NS-37766, NS-40144, and NS-40810 from the National Institute of Health.

### BACKGROUND OF THE INVENTION

1. Field of the Invention

The present invention relates generally to the fields of molecular biology and medicine. More particularly, it concerns materials and methods for the inhibition of inflammatory and cytokine-mediated responses.

2. Description of Related Art

Excessive production of nitric oxide (NO) has been implicated in neuronal cell death and demyelination in a number of central nervous system diseases such as multiple sclerosis, Parkinson's, Alzheimer's, Krabbe's disease, bacterial/viral infections, cerebral ischemia and spinal cord injury (SCI) and in an inherited metabolic disorder of peroxisomes, X-Adrenoleukodystrophy (Dawson et al., 1993; Koprowski et al., 1993; Bo et al., 1994; Vodovotz et al., 1996; Wada et al., 1998a; Wada et al., 1998b; Akiyama et al., 2000; Gilg et al., 2000; Satake et al., 2000; Giri et al., 2002).

Of the three isoforms of nitric oxide synthase (NOS), two isoforms are calcium dependent and constitutively expressed (neuronal, nNOS & endothelial, eNOS). The third is a calcium independent and inducible isoform (iNOS). iNOS, once induced in response to a number of stress inducing factors such as pro-inflammatory cytokines, bacterial/viral components etc. produces high amounts of NO (Simmons and Murphy, 1992; Zielasek et al., 1992). The pathologically high levels of NO produced by iNOS in the CNS are associated with inhibition of mitochondrial functions, rapid glutamate release from both astrocytes and neurons, and excitotoxic death of neurons (Leist et al., 1997; Sequeira et al., 1997; Bal-Price and Brown, 2001). iNOS expression in reactive astrocytes has been implicated in the development of post-traumatic spinal cord cavitation and neurological impairment (Matsuyama et al., 1998; Suzuki et al., 2001). U.S. Pat. No. 6,511,800 describes methods for treating nitric oxide mediated diseases. Strategies for iNOS inhibition to improve neurological outcome are an active area of investigation in neuroinflammatory diseases.

Previously, an involvement of sphingolipids such as ceramide and psychosine in regulation of cytokine-mediated iNOS expression has been observed (Pahan et al., 1998b; Fern, 2001; Giri et al., 2002). However, no involvement of glycosphingolipids (GSL) such as Lactosylceramide (LacCer) in the regulation of cytokine-mediated iNOS gene expression has been demonstrated.

Reactive astrogliosis is another manifestation of trauma to the central nervous system (CNS). Traumatic injury to the adult CNS results in a rapid inflammatory response by the resident astrocytes, characterized mainly by hypertrophy, proliferation and increased expression of glial fibrillary acidic protein (GFAP) resulting in reactive astrogliosis (Mucke and Eddleston, 1993; Ridet et al., 1997; Dihne et al., 2001; Kernie et al., 2001). The glial scar formed as the result of gliosis has been suggested to be an attempt made by the CNS to restore homeostasis through isolation of the damaged area. Although many reasons have been put forward to explain the obvious lack of CNS regeneration following injury/neurotrauma, the robust formation of the glial scar may also interfere with any subsequent neural repair or CNS axonal regeneration (Ridet et al., 1997; Steeves and Tetzlaff, 1998). Thus, considerable effort is being directed toward understanding the mechanisms involved in astrocyte proliferation and reactivity in order to design therapeutic approaches to modulate gliosis which seems to be necessary for restoring homeostasis following and insult to the CNS at the same time is an impediment to neuronal recovery and axonal regeneration.

Following CNS injury, tumor necrosis factor-alpha (TNFα) has been identified as one of the first cytokines to appear following CNS injury and has been implicated in exacerbation of CNS injury by causing apoptosis of neurons and oligodendrocytes, recruitment of peripheral immune cells by way of upregulation adhesion molecule expression. TNFα induces proliferation of both primary astrocytes (Barna et al., 1990; Selmaj et al., 1990) and human astroglioma cell lines (Lachman et al., 1987; Bethea et al., 1990) and has also been tightly linked with the reactive transformation of astrocytes. While the activation of sphingomyelinases and the resulting sphingomyelin-ceramide pathway has been closely linked with TNFα-induced apoptosis in numerous cell types, TNFα is also known to activate sphingosine kinase resulting in sphingosine-1-phosphate (S1P) generation that is mitogenic for various cell types (Pettus et al., 2003). It is well accepted that ceramide once generated in a cell can be converted into other metabolites which could exert antagonistic effects. These antagonistic effects are regulated by enzymes that interconvert ceramide into its metabolites and vice-versa, thus leading to the proposal of a ‘sphingolipid rheostat’ which is critical in determining cell fate (Cuvillier et al., 2000). According to this hypothesis, it is not the absolute but the relative amounts of these antagonistic metabolites that regulate cell fate and might shift the balance from cell death to survival and vice-versa.

As stated above, the symptoms associated with neuroinflammatory diseases and injury to the central nervous system are severe, and limited approaches to inhibiting neuroinflammatory responses currently exist. These factors illustrate the need for new approaches for inhibiting neuroinflammatory responses.

### SUMMARY OF THE INVENTION

The present invention overcomes deficiencies in the art by demonstrating that inhibitors of glycosphingolipid metabolism, preferrably inhibitors of glucosylceramide synthase and/or GalT-2, can be used to treat and/or prevent inflammatory and cytokine mediated responses such as neuroinflammatory responses associated with injury to the central nervous system.

An aspect of the invention involves a method of treating a nitric oxide or cytokine mediated disorder in a subject, comprising administering a biologically effective amount of a glycosphingolipid inhibitor. The glycosphingolipid inhibitor may be an inhibitor of glucosylceramide synthase or GalT-2. In certain preferred embodiments, the subject is a mammal, preferably a human. The biologically effective amount may be administered to said mammal. The nitric oxide or cytokine mediated disorder may be sickle cell anemia, infections by gram-positive bacteria, common cold, vascular disorders, endothelial disorders, recreational drug abuse, or neurotoxin poisoning. In certain embodiments, the nitric oxide or cytokine mediated disorder is an inflammatory disease. The inflammatory disease may be stroke, meningitis, X-adenoleukodystrophy (X-ALD) or other leukodystrophies, multiple sclerosis, Alzheimer's disease, cancer, lupus, Landry-Guillain-Barre-Strohl syndrome, brain trauma, spinal cord disorders, viral encephalitis, acquired immunodeficiency disease (AIDS)-related dementia, septic shock, adult respiratory distress syndrome, myocarditis, amyotrophic lateral sclerosis, cystic fibrosis, ischemia or ischemia-reperfusion injury, arthritis or an autoimmune disease. The inflammatory disease may be an inflammatory bowel disease, an inflammatory lung disorder, an inflammatory eye disorder, a chronic inflammatory gum disorder, a chronic inflammatory joint disorder, a skin disorder, a bone disease, a heart disease or kidney failure. In certain preferred embodiments, the inflammatory disease is a neuroinflammatory disorder. The neuroinflammatory disorder may be Alzheimer's disease, Parkinson's disease, Landry-Guillain-Barre-Strohl syndrome, multiple sclerosis, stroke, Alzheimer's disease, viral encephalitis, cerebral palsy, acquired immunodeficiency disease (AIDS)-related dementia amyotrophic lateral sclerosis, brain trauma, spinal cord disorders, reactive astrogliosis or spinal cord trauma.

The glycosphingolipid inhibitor may be, in certain preferred non-limiting embodiments, a PDMP derivative, N-butyldeoxynojirimycin, Miglustat, or PDMP. The PDMP derivative may be D-threo-3′,4′-ethylenedioxy-1-phenyl-2-palmitoylamino-3-pyrrolidino-1-propanol or D-threo-4′-hydroxy-1-phenyl-2-palmitoylamino-3-pyrrolidino-1-propanol. The glycosphingolipid inhibitor may be an inhibitor of sphingosine kinase or sphingosine-1-phosphate phosphatase. In certain preferred embodiments, the PDMP is in a pharmaceutically acceptable excipient. The PDMP may be administered with a second pharmaceutical preparation. The second pharmaceutical preparation may enhances intracellular cAMP. The second pharmaceutical preparation may be Rolipram or GM1. The second pharmaceutical preparation may comprise an inhibitor of mevalonate synthesis, an inhibitor of the farnesylation of Ras, an antioxidant, an enhancer of intracellular cAMP, an enhancer of protein kinase A (PKA), an inhibitor of NF-κ.beta. activation, an inhibitor of Ras/Raf/MAP kinase pathway, an inhibitor of mevalonate pyrophosphate decarboxylase or an inhibitor of farnesyl pyrophosphate.

Another aspect of the present invention involves a method of making a glycosphingolipid inhibitor comprising: providing in a cell or cell-free system a glycosphingolipid enzyme polypeptide, contacting the glycosphingolipid enzyme with a candidate substance, selecting an inhibitor of the glycosphingolipid enzyme by assessing the effect of said candidate substance on glycosphingolipid enzyme activity, and manufacturing the inhibitor. The glycosphingolipid enzyme may be glucosylceramide synthase or GalT-2. Said candidate substance may be a protein, a nucleic acid or an organo-pharmaceutical. The protein may be an antibody that binds immunologically to glucosylceramide synthase or GalT-2. The nucleic acid may be an antisense molecule. The nucleic acid is an siRNA molecule. Said assessing may comprise evaluating production of LacCer or GluCer.

Another aspect of the present invention involves a method of inhibiting an inflammatory or cytokine-mediated response in a cell comprising administering to the cell an effective amount of an inhibitor manufactured according to any one of the methods disclosed herein, to inhibit the enzymatic activity of glucosylceramide synthase or GalT-2. Said inhibitor may inhibit the enzymatic activity of glucosylceramide synthase and GalT-2. Said cell may be in a mammal, preferably in a human. Said cell may be a cell of the central nervous system or the peripheral nervous system. Said cell may be a neuron or an astrocyte. The inhibitor may be a protein, a nucleic acid or an organo-pharmaceutical. The protein may be an antibody that binds immunologically to glucosylceramide synthase or GalT-2. The nucleic acid may be an antisense molecule, a short interfering nucleic acid (siNA), or an siRNA.

It is contemplated that any embodiment discussed in this specification can be implemented with respect to any method or composition of the invention, and vice versa. Furthermore, compositions of the invention can be used to achieve methods of the invention.

“Ischemia-reperfusion injury” may be, in a non-limiting embodiment, the result damage to an organ that is stored or transplanted into a subject. The subject is preferrably a mammal, more preferrably human. In preferred non-limiting embodiments, the organ is a heart, kidney, liver, or pancreas.

The use of the word “a” or “an” when used in conjunction with the term “comprising” in the claims and/or the specification may mean “one,” but it is also consistent with the meaning of “one or more,” “at least one,” and “one or more than one.”

The use of the term “or” in the claims is used to mean “and/or” unless explicitly indicated to refer to alternatives only or the alternatives are mutually exclusive.

Throughout this application, the term “about” is used to indicate that a value includes the inherent variation of error for the device, the method being employed to determine the value, or the variation that exists among the study subjects.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-B: LacCer regulates the LPS/IFNγ-induced NO production and iNOS gene expression in rat primary astrocytes. Effect of PDMP (10, 25 and 50 μM) on NO production and the induction of iNOS mRNA and protein expression was examined after 6 h (for iNOS mRNA level) or 24 h (for iNOS protein and NO levels) after LPS/IFNγ (1 μg/ml; 10 U/ml) stimulation (FIG. 1A). The cells were pretreated with PDMP for 0.5 h before LPS/IFNγ stimulation. The effect of LacCer on PDMP-mediated inhibition of iNOS gene expression in astrocytes was also examined. The cells were pretreated with PDMP (50 μM) and/or LacCer (5 and 10 μM) for 0.5 h before LPS/IFNγ stimulation. NO production and iNOS mRNA and protein levels were quantified, 6 h and 24 h after LPS/IFNγ stimulation, respectively (FIG. 1B). Levels of GAPDH were used as an internal standard for mRNA levels. The procedures for measurement of mRNA and of protein and NO are described in Example 1. Data are represented as mean±S.D from three independent experiments. ***p<0.001 in (FIG. 1A & FIG. 1B) as compared with unstimulated control; **p<0.01 and #p<0.001 in (FIG. 1A) as compared with LPS/IFNγ stimulated cells. #p<0.001 in (FIG. 1B) as compared with LPS/IFNγ-stimulated cells; *p<0.001 and **p<0.001 in (FIG. 1B) as compared with PDMP treated cells.

FIGS. 2A-E: Effect of various metabolites of the glycosphingolipid pathway on PDMP-mediated inhibition of LPS-induced NO production. Primary astrocytes were pretreated with PDMP and Glucer (FIG. 2A), GalCer (FIG. 2B), GM₁ (FIG. 2C), GM₃ (FIG. 2D) or GD₃ (FIG. 2E) all at individual concentrations of 5 and 10 μM for 0.5 h prior to stimulation with LPS/IFNγ. NO production was assayed at 24 h following LPS/IFNγ stimulation as described in FIGS. 1A-B.

FIGS. 3A-E: The effect of LPS/IFNγ stimulation on the biosynthesis of LacCer. Primary astrocytes were treated with [¹⁴C]galactose overnight. Upon pretreatment with PDMP 0.5 h before LPS/IFNγ stimulation, cells were harvested at the time points indicated and LacCer was analyzed by HPTLC as described in Example 1 (FIG. 3A). The enzyme activity of LacCer synthase (GalT-2) was assayed as described in Example 1 using cell lysates derived from cells stimulated with LPS/IFNγ for various durations as shown (FIG. 3B). For the silencing of GalT-2 gene, the cells were transfected with either GalT-2 antisense DNA oligomer or its sequence-scrambled DNA oligomer (Scr) as described in Example 1. At 48 h after transfection the protein levels of GalT-2 as well as [¹⁴C]LacCer synthesis was done as described earlier (FIG. 3C). 48 hrs following transfection GalT-2 protein level were analysed by immunoblot analysis and [¹⁴C]LacCer synthesis was examined in transfected and non-transfected cells (FIG. 3C). 48 hrs following transfection with AS oligonucleotides, cells were stimulated with LPS/IFNγ and NO production (FIG. 3D) and the mRNA levels of iNOS, TNFα and IL-1β (FIG. 3E) were measured as described earlier. Data are represented as mean±S.D of three independent experiments. ***p<0.001 in (FIG. 3A) and ***p<0.001 in (FIG. 3B) compared with unstimulated control. ***p<0.001 in (FIG. 3D) compared with stimulated, untransfected cells; #p<0.001 in (FIG. 3D) compared with transfected cells without LacCer.

FIGS. 4A-C: LacCer-mediated regulation of LPS/IFNγ-induced iNOS gene expression is ROS mediated. Effect of NAC (5, 10 mM) and PDTC (50 and 100 μM) pretreatment 1 h before LPS/IFN-stimulation was analyzed on NO production and the induction of iNOS mRNA and protein expression was examined after 6 h (for iNOS mRNA) or 24 h (for iNOS protein and NO levels) after LPS/IFNγ (1 μg/ml; 10 U/ml) stimulation (FIG. 4A). The effect of LacCer on NAC- and PDTC-mediated inhibition of iNOS gene expression was also analyzed. The cells were pretreated with NAC (10 mM) or PDTC (100 μM) for 1 h before LPS/IFNγ and LacCer-stimulation. NO production, iNOS protein and mRNA levels (FIG. 4B) were quantified at 24 h and 6 h after LPS/IFNγ stimulation, respectively. NAC and PDTC were pretreated 1 h and PDMP/LacCer 0.5 h before LPS/IFNγ stimulation following which NO production and iNOS protein and mRNA levels were analyzed (FIG. 4C). Data are represented mean±S.D of three independent experiments. ***p<0.001 in (FIG. 4A) compared with LPS/IFN-stimulated cells without NAC or PDTC.

FIGS. 5A-G: The involvement of small GTPase Ras and ERK1/2 in LacCer-mediated regulation of LPS-induced iNOS gene expression in primary astrocytes. Dominant negative Ras (DN-Ras) was transiently transfected in primary astrocytes followed by stimulation with LPS/IFNγ and/or LacCer. NO production and iNOS protein and mRNA levels analyzed as described previously (FIG. 5A). Constitutively active Ras (CA-Ras) was transiently transfected followed by PDMP pretreatment 0.5 h before LPS/IFNγ stimulation. NO production, iNOS protein and mRNA expression is shown (FIG. 5B). Following transient transfection with DN-Ras and CA-Ras, synthesis of [¹⁴C]LacCer upon LPS/IFNγ stimulation of primary astrocytes was analyzed as described in Example 1 (FIG. 5C). Ras activation was examined using GST tagged Raf-1 Ras binding domain (GST-RBD) as described in Example 1. Ras activation was checked following LPS/IFNγ stimulation for different durations of time. Following pretreatment with LacCer and/or PDMP (50 μM) for 0.5 h followed by LPS/IFNγ stimulation for 5 min, cell lysates were used to assay levels of activated Ras which is also represented as a graph following densitometric analysis of the autoradiograph (FIG. 5D). Following pretreatment with NAC (10 mM) or PDTC (100 μM) for 1 h followed by LacCer stimulation for 5 min, cell lysates were used to assay levels of activated Ras which is also represented as a graph following densitometry of the autoradiograph (FIG. 5E). ERK1/2 activation was assayed upon pretreatment of cells with LacCer and/or PDMP for 0.5 h followed by stimulation with LPS/IFNγ for 20 min, immunoblot analysis using anti-phosphorylated ERK1/2 antibodies as described in Example 1 (FIG. 5F). To examine MEK/ERK pathway involvement, upon pretreatment for 0.5 h with PD98059, (a MEK1/2 inhibitor), followed by stimulation with LPS/IFNγ for 24 h, NO production and iNOS protein levels were assayed (FIG. 5G).

FIGS. 6A-C: Involvement of LacCer in LPS/IFNγ-mediated NF-κB activation and iNOS gene expression. 24 h after transient transfection of cells with κB-luciferase gene construct, cells were pre-treated with PDMP, 0.5 h prior to stimulation with LPS/IFNγ. The cellular luciferase activity was measured as described in Example 1 (FIG. 6A). The NF-κB DNA binding activity was detected by gel shift assay using 10 μg of nuclear extract from cells pretreated for 0.5 h with LacCer and/or increasing doses of PDMP followed by stimulation with LPS/IFNγ for 45 min (FIG. 6B). The cytoplasmic extract was used to detect the levels of phosphorylated IκB and total IκB levels by immunoblot using antibodies against phosphorylated IκB and total IκB (FIG. 6C). Data are represented as mean±SD of three independent experiments

FIGS. 7A-P: Histology and myelin content examination of spinal cord sections from the lesion epicenter of Sham and SCI rats. (FIGS. 7A-H) shows H&E examination of spinal cord sections from VHC-treated Sham (FIG. 7A), VHC-treated SCI (FIG. 7B) and PDMP-treated Sham (FIG. 7C) and SCI (FIGS. 7D-H). (FIGS. 7I-P) shows LFB-PAS staining for myelin in VHC-treated Sham (FIG. 7I) SCI (FIG. 7J) and PDMP-treated Sham (FIG. 7K) and SCI (FIGS. 7L-P) 24 h post-SCI. PDMP was administered i.p at the indicated time (10 min, 30 min, 1 h, 2 h and 12 h) following SCI and tissue sections were extracted and analyzed at Day 1 (24 h) post-SCI.

FIGS. 8A-M: Locomotor function of PDMP- and VHC-treated rats post-SCI. BBB locomotor scores of PDMP- and VHC-treated SCI animals at various days after contusion injury (FIG. 8A). 21 represents normal locomotion, 0 represents no observable movement. Increase in BBB score reflects gain in hind limb function and recovery. Histology and myelin content examination of spinal cord sections from the lesion epicenter of Sham and SCI rats at Days 2 and 3 post-SCI. (FIGS. 8B-D) shows H&E examination of spinal cord sections from VHC-treated Sham (FIG. 8B), VHC-treated SCI at Day 2 (FIG. 8C) and VHC-treated SCI at Day 3 post SCI (FIG. 8D). (FIGS. 8E-G) shows H&E examination of spinal cord sections from PDMP-treated Sham (FIG. 8E), PDMP-treated SCI at Day 2 (FIG. 8F) and PDMP-treated SCI at Day 3 post SCI (FIG. 8G). (FIGS. 8H-J) shows LFB-PAS staining for myelin in VHC-treated Sham (FIG. 8H), VHC-treated SCI at Day 2 (FIG. 8I) and VHC-treated SCI at day 3 post-SCI (FIG. 8J). (FIGS. 8K-M) shows LFB-PAS staining for myelin in PDMP-treated Sham (FIG. 8K), PDMP-treated SCI at Day 2 (FIG. 8L) and PDMP-treated SCI at Day 3 post-SCI (FIG. 8M). Dose 1 of PDMP was administered 10 min post-SCI, dose 2 at Day 1 (24 h), Dose 3 at Day 2 (48 h) and Dose 4 at Day 3 (72 h) post-SCI. Tissue sections were extracted and analyzed at Day 2 (48 h) and Day 3 (72 h) post-SCI. Data are represented mean±S.D. ***p<0.001 in (FIG. 8A) compared with VHC-treated SCI at day 3, #p<0.001 in (FIG. 8A) as compared with VHC-treated SCI at Day 15 post-SCI.

FIGS. 9A-N: iNOS mRNA and protein expression at the lesion epicenter following SCI. iNOS mRNA levels were quantified by real time PCR analysis (FIG. 9A) and protein levels by immunoblot analysis (FIG. 9B) from RNA and protein samples derived from spinal cords sections of VHC- or PDMP-treated Sham operated or SCI rats. Data are represented as mean±SD. ***p<0.001 in (FIG. 9A) as compared to VHC treated Sham; #p<0.001 as compared to VHC treated 12 h. Double immunofluorescence staining of spinal cord sections from the lesion epicenter for iNOS/GFAP co-localization. Immunofluorescent microscopy images of spinal cord sections from Sham and SCI rats, stained with antibodies to iNOS (green) and GFAP (red) as described in Example 1. (FIGS. 9C-E) shows GFAP (FIG. 9C), iNOS (FIG. 9D) and their co-localization (FIG. 9E) in VHC-treated Sham. (FIGS. 9F-H) shows GFAP (FIG. 9F), iNOS (FIG. 9G) and their co-localization (FIG. 9H) in VHC-treated SCI. (FIGS. 9I-K) shows GFAP (FIG. 9I), iNOS (FIG. 9J) and their co-localization (FIG. 9K) in PDMP treated Sham. (FIG. 9L-N) shows GFAP (FIG. 9L), iNOS (FIG. 9M) and their co-localization (FIG. 9N) in PDMP-treated SCI rats.

FIGS. 10A-J: TNFα and IL-1β mRNA and protein expression at the lesion epicenter following SCI. TNFα (FIG. 10A) and IL-1β (FIG. 10B) mRNA levels were quantified by real time PCR analysis at various durations post-SCI. Immunofluorescent microscopy images of spinal cord sections from Sham and SCI rats, stained with antibodies to TNFα (FIGS. 10C-F) showing VHC-treated Sham (FIG. 10C), VHC-treated SCI (FIG. 10D) and PDMP-treated Sham (FIG. 10E) and −SCI (FIG. 10F) extracted 1 h post-SCI. Immunofluorescent microscopy images of spinal cord sections from Sham and SCI rats, stained with antibodies to IL-1β as described in Example 1 (FIGS. 10G-J) shows VHC-treated Sham (FIG. 10G), VHC-treated SCI (FIG. 10H) and PDMP-treated Sham (FIG. 10I) and −SCI (FIG. 10J) extracted 4 h post-SCI. Data are represented as mean±SD. ***p<0.001 in (A and B) as compared to VHC treated Sham; #p<0.001 in (FIG. 10A) as compared to VHC treated 1 h; #p<0.001 in (FIG. 10B) as compared to VHC treated 4 h.

FIGS. 11A-L: Double immunofluorescence staining of spinal cord sections from the lesion epicenter for TUNEL positive nuclei and Neuron specific marker (NeuN): Immunofluorescent microscopy images of spinal cord sections taken 24 h post-SCI from Sham and SCI rats stained for TUNEL positive cells (green) using APOPTAG detection kit and antibodies to a neuron specific marker NeuN (red) as described in Example 1. (FIGS. 11A-C) shows NeuN (FIG. 11A), TUNEL (FIG. 11B) and their co-localization (FIG. 11C) in VHC-treated Sham. (FIGS. 11D-F) shows NeuN (FIG. 11D), TUNEL (FIG. 11E) and their co-localization (FIG. 11F) in VHC-treated SCI. (FIGS. 11G-H) shows NeuN (FIG. 11G), TUNEL (FIG. 11H) and their co-localization (FIG. 11I) in PDMP-treated Sham. (FIGS. 11J-L) shows NeuN (FIG. 11J), TUNEL (FIG. 11K) and their co-localization (FIG. 11L) in PDMP-treated SCI rats.

FIG. 12: Schematic representation of the model for LacCer mediated regulation of LPS/IFNγ-induced iNOS gene expression in rat primary astrocytes.

FIGS. 13A-F: LacCer regulates TNFα-induced proliferation and GFAP gene expression in rat primary astrocytes. Effect of TNFα on astrocyte proliferation, assayed by BrdU incorporation, was examined 18 h following stimulation with increasing concentrations of TNFα (0, 0.1, 1 and 5 ng/ml) (FIG. 13A). Effect of PDMP (10, 25 and 50 μM) on cell proliferation was assayed. The cells were pretreated with PDMP for 0.5 h before TNFα (1 ng/ml) treatment (FIG. 13B). The mitogenic effect of increasing concentration of LacCer (1, 5 and 10 μM) and GluCer (1, 5 and 10 μM) was assayed 18 h following stimulation with LacCer and GluCer by BrdU incorporation (FIG. 13C). The ability of LacCer or GluCer to reverse PDMP-mediated inhibition of TNFα-induced cell proliferation was examined. The cells were pretreated with PDMP (25 μM) and/or LacCer (10 μM)/GluCer (10 μM) for 0.5 h before TNFα-stimulation (FIG. 13D). The involvement of PDMP and LacCer in TNFα-induced GFAP expression was examined. PDMP (25 μM) and/or LacCer (5 μM) were pretreated for 0.5 h followed by stimulation with TNFα (1 ng/ml). GFAP mRNA levels were examined by real time PCR analysis 8 h following stimulation with TNFα (FIG. 13E). GFAP mRNA levels were normalized with GAPDH mRNA levels. GFAP protein levels were detected 18 h following TNFα-stimulation by immunoblot analysis (FIG. 13F).The procedures for real time PCR and protein analysis are described in Example 1. Data are represented as mean±S.D from three independent experiments. ***p<0.001 in (FIG. 13A, FIG. 13B, FIG. 13C, FIG. 13D and FIG. 13E) as compared with unstimulated control; ^{#p<}0.001 in (FIG. 13B and FIG. 13D) as compared with TNFα-stimulated, ^{@p<}0.001 in (FIG. 13D) as compared to PDMP-treated, ^{#p<}0.001 in (FIG. 13E) as compared to unstimulated, ^{&}p<0.001 in (FIG. 13E) as compared to TNFα-stimulated and ^{%}p<0.001 in (FIG. 13E) as compared to PDMP-treated.

FIGS. 14A-D: Effect of various metabolites of the glycosphingolipid pathway on PDMP-mediated inhibition of TNFα-induced astrocyte proliferation. Primary astrocytes were pretreated with PDMP and/or GalCer (FIG. 14A), GM₁ (FIG. 14B), GM₃ (FIG. 14C) and GD₃ (FIG. 14D) all at individual concentrations of 1, 5 and 10 μM for 0.5 h prior to stimulation with TNFα. Cell proliferation was assayed at 18 h following TNFα-stimulation as described in FIGS. 13A-F.

FIGS. 15A-F: The effect of TNFα-stimulation on the biosynthesis of LacCer. Primary astrocytes were treated with [¹⁴C]galactose overnight. Following TNFα-stimulation (1 ng/ml), cells were harvested at the time points indicated and [¹⁴C]LacCer was analyzed by HPTLC as described in Example 1 (FIG. 15A). The enzyme activity of LacCer synthase (GalT-2) was assayed as described in Example 1 using cell lysates derived from cells stimulated with TNFα for various durations as shown (FIG. 15B). For the silencing of GalT-2 gene, the cells were transfected with either GalT-2 antisense DNA oligomer or its sequence-scrambled DNA oligomer (Scr) as described in Example 1. At 48 h after transfection the protein levels of GalT-2 as well as [¹⁴C]LacCer synthesis was done as described earlier (FIG. 15C). 48 hrs following transfection with AS oligonucleotides, cells were stimulated with TNFα and cell proliferation (FIG. 15D), GFAP mRNA (FIG. 15E) and protein (FIG. 15F) levels were assayed as described earlier. Data are represented as mean±S.D of three independent experiments. ***p<0.001 in (FIG. 15A) as compared with unstimulated control. ***p<0.001 in (FIG. 15C, FIG. 15D and FIG. 15E) compared with stimulated, untransfected cells; #p<0.001 in (FIG. 15D and FIG. 15E) compared with AS-transfected cells without LacCer.

FIGS. 16A-F: The involvement of small GTPase Ras and ERK1/2 in LacCer mediated regulation of TNFα-induced proliferation and GFAP gene expression in primary astrocytes. Dominant negative Ras was transiently co-transfected with pEGFP (transfection marker) in primary astrocytes followed by stimulation with TNFα and/or LacCer. Cell cycle status of GFP gated cell population was assayed by FACS analysis (FIG. 16A) and GFAP mRNA and protein expression (FIG. 16B) was assayed in DN-Ras and mock transfected primary astrocytes. Ras activation was examined using GST tagged Raf-1 Ras binding domain as described in Example 1. Following pretreatment with LacCer and/or PDMP (25 μM) for 0.5 h followed by TNFα-stimulation for 5 min, cell lysates were used to assay levels of activated Ras which is represented as a graph following densitometric analysis of the autoradiograph (FIG. 16C). To examine MEK/ERK pathway involvement, upon pretreatment for 0.5 h with PD98059 (30 μM) and or LacCer (10 μM), (a MEK1/2 inhibitor), followed by stimulation with TNFα for 18 h, cell proliferation was assayed (FIG. 16D). ERK1/2 activation was assayed upon pretreatment of cells with LacCer and/or PDMP or PD98059 for 0.5 h followed by stimulation with TNFα for 20 min, immunoblot using phosphorylated ERK1/2 as described in Example 1 (FIG. 16E). To examine MEK/ERK pathway involvement, upon pretreatment for 0.5 h with PD98059, (a MEK1/2 inhibitor), followed by stimulation with TNFα, GFAP mRNA and protein levels were assayed (FIG. 16F).

FIGS. 17A-H: The involvement of PI-3K in TNFα-induced cell proliferation and GFAP gene expression in primary astrocytes. Pretreatment with LY294002, a specific PI-3K inhibitor, for 0.5 h was followed by stimulation with TNFα and cell proliferation was assayed (FIG. 17A). A kinase deficient PI-3K catalytic subunit, p110Δkin, was transiently co-transfected with pEGFP (transfection marker) in primary astrocytes followed by stimulation with TNFα and/or LacCer. Cell cycle status of GFP gated cell population was assayed by FACS analysis (FIG. 17B). Following pretreatment with LY294002 and TNFα-simulation, [¹⁴C]LacCer production (FIG. 17C) and GalT-2 enzyme activity (FIG. 17D) was assayed at different time points as described in material and methods . Ras activation was examined using GST tagged Raf-1 Ras binding domain as described in Example 1. Following pretreatment with LacCer and/or LY294002 (30 μM) for 0.5 h followed by TNFα-stimulation, cell lysates were used to assay levels of activated Ras which is represented as a graph following densitometric analysis of the autoradiograph (FIG. 17E). To examine MEK/ERK pathway involvement, upon pretreatment for 0.5 h with LY294002 (30 μM) and or LacCer (10 μM), ERK1/2 activation was assayed by immunoblot using phosphorylated ERK1/2 as described in Example 1 (FIG. 17F). To examine PI-3K involvement in GFAP gene expression, upon pretreatment for 0.5 h with LY294002, followed by stimulation with TNFα, GFAP mRNA (FIG. 17G) and protein levels were assayed (FIG. 17H).

FIGS. 18A-J: TNFα-induced activation of PI-3K resulting in astrocyte proliferation is mediated by S1P. Increasing concentrations of S1P induce proliferation of primay astocytes (FIG. 18A). Pretreatment with increasing doses of dimethylsphingosine (10, 30 and 50 μM) inhibits TNFα-induced proliferation (FIG. 18B). Exogenous supplementation of S1P reverses DMS mediated inhibition of TNFα-induced proliferation and GFAP expression however SIP has no effect on LY mediated inhibition (FIG. 18C and FIG. 18E). Furthermore, exogenous supplementation of S1P could not reverse PDMP and PD98059-mediated inhibition of TNFα-induced astrocyte proliferation and GFAP expression (FIG. 18D and FIG. 18F). DMS or LY294002 pretreatment inhibits TNFα-induced ERK1/2 expression, however exogenous supplementation of S1P only reverses DMS-induced inhibition and not LY294002 (FIG. 18G). PDMP or PD98059 pretreatment inhibits TNFα-induced ERK1/2 activation and neither is reversed by S1P supplementation (FIG. 18H). Pretreatment with DMS (30 mM) for 0.5 h followed by TNFα-stimulation inhibitis PI-3K activity assayed as described in Example 1. However exogenous supplementation of S1P reverses DMS mediated inhibiton of PI-3K activity. Exogenously supplemented Laccer has no effect on DMS mediated inhibiton of TNFα-induced activity of PI-3K (FIG. 18I). DMS pretreatment for 0.5 h followed by TNFα-stimulation inhibits ras activation which is reversed by exogenous supplementation of S1P (FIG. 18J).

FIGS. 19A-C: ERK1/2 activation and GFAP mRNA and protein expression at the lesion epicenter following SCI. phosphorylated ERK1/2 levels were assayed by immunoblot analysis from protein samples derived from spinal cords sections of vehicle (VHC) or PDMP-treated Sham operated or SCI rats. The ratio of pERK/ERK is depicted as well (FIG. 19A). GFAP mRNA levels were quantified by real time PCR analysis (FIG. 19B) and protein levels by immunoblot analysis (FIG. 19C) from RNA and protein samples derived from spinal cords sections of vehicle (VHC) or PDMP treated Sham operated or SCI rats. Data are represented as mean±SD. ***p<0.001 in (FIG. 19A) as compared to VHC treated Sham; #p<0.001 as compared to VHC treated 12 h.

FIGS. 20A-L: Double immunofluorescence staining of spinal cord sections from the lesion epicenter for pERK/GFAP co-localization. Immunofluorescent microscopy images of spinal cord sections from Sham and SCI rats, stained with antibodies to pERK (green) and GFAP (red) as described in Example 1. (FIGS. 20A-C) shows GFAP (FIG. 20A), pERK (FIG. 20B) and their co-localization (FIG. 20C) in VHC-treated Sham. (FIGS. 20D-F) shows GFAP (FIG. 20D), pERK (FIG. 20E) and their co-localization (FIG. 20F) in VHC-treated SCI. (FIGS. 20G-I) shows GFAP (FIG. 20G), pERK (FIG. 20H) and their co-localization (FIG. 20I) in PDMP treated Sham. (FIGS. 20J-L) shows GFAP (FIG. 20J), pERK (FIG. 20K) and their co-localization (FIG. 20L) in PDMP treated SCI rats.

### DESCRIPTION OF ILLUSTRATIVE EMBODIMENTS

The present invention overcomes deficiencies in the art by demonstrating that inhibitors of glycosphingolipid metabolism, preferrably inhibitors of glucosylceramide synthase and/or GalT-2, can be used to treat and/or prevent inflammatory and cytokine mediated responses such as neuroinflammatory responses associated with injury to the central nervous system.

A. Lactosylceramide (LacCer)

Inhibitors of lactosylceramide (LacCer) synthesis may be used in preferred embodiments of the present invention to treat and/or prevent inflammatory and cytokine mediated responses. LacCer is a glycosphingolipid (GSL) which has been implicated in several important cellular functions including intracellular signaling and the progression of certain forms of cancer. In endothelial tissues and aortic muscle cells, LacCer is associated with the production of superoxide radicals. In umbilical vein endothelial cells, LacCer stimulated the endogenous generation of superoxide radicals (Bhunia et al., 1998). It has been hypothesized that these superoxide radicals are responsible for the proliferation of human aortic smooth muscle cells (Chatterjee, 1998).

LacCer is synthesized from ceramide. GSL biosynthesis is initiated by transfer of glucose from UDP-glucose onto ceramide by the action of glucosylceramide synthase to form glucosylceramide (GluCer). LacCer is generated from GluCer and UDP-galactose by the action of LacCer synthase (also referred to as “lactosylceramide synthase” or “GalT-2”). LacCer is a precursor for complex GSL including gangliosides.

B. GSL Biosynthesis Inhibitors

Inhibitors of GSL biosynthesis, preferably inhibitors of GluCer and/or LacCer synthesis, may be used with the present invention to inhibit inflammatory and cytokine-mediated responses. Inhibitors of GluCer and/or LacCer synthesis include N-butyldeoxynojirimycin, N-butyldeoxygalactonojirimycin, and N-nonyldeoxynojirimycin; 1-phenyl-2-decanoylamino-3-morpholino-1-propanol, D-threo-1-phenyl-2-decanoylamino-3-morpholino-1-propanol (PDMP), and structurally related analogues thereof. Other compounds that inhibit GluCer and LacCer synthesis may also be used with the present invention.

Inhibition of glucosylceramide synthesis can also be achieved by “knockdown” of the expression of the glucosylceramide synthesis gene using techniques including antisense, small interfering nucleic acids (siNA), and small inhibitory RNA (siRNA). Techniques to “knockdown” the expression of a gene of interest generally include exposing a cell to a specific nucleic acid sequence, and the nucleic acid sequence may be delivered via a pharmaceutically acceptable carrier (e.g., liposomes) or via a viral delivery system (e.g., adenoviral delivery). A combination of any of the above approaches can be used.

### 1. D-threo-1-Phenyl-2-decanoylamino-3-morpholino-1-propanol.HCI (PDMP or D-threo PDMP)

PDMP is a glucosylceramide synthase and lactosylceramide synthase inhibitor. The molecular formula for PDMP is C₂₃H₃₈N₂₀₃HCI. D-PDMP includes a molecular weight of 427.1 and is soluble in water. The chemical formula for PDMP

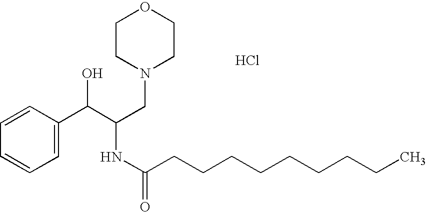

is:

It is contemplated that PDMP can be used alone, or in combination with the other compounds disclosed in the specification, to treat or prevent inflammatory diseases and conditions.

PDMP specifically inhibits the glucosulceramide synthase and GalT-2 enzymes, which are necessary for glucosylceramide biosynthesis. PDMP thus reduces intracellular content of all GSL which are produced starting with glucosylceramide (Inokuchi and Radin, 1987).

PDMP has been observed to affect several cellular events. PDMP can suppress the extension of neurite (Uemura et al., 1991; Mendez-Otero and Cavalcante, 2003), and it has also been reported to suppress synaptic function, an effect which was inhibited by addition of the ganglioside GQ1b (Mizutani et al., 1996). In contrast with the findings of the present invention, PDMP increased IL-1β stimulated nitric oxide release in rat aortic vascular smooth muscle cells (Weber et al., 1998).

PDMP may also be useful for treating cancer. Because PDMP can also reduce the ability of neuroblastoma tumours to escape from host immune destruction, and this effect appears to be due to the ability of PDMP to block the shedding of gangliosides by cancerous cells (Li et al., 1996) and by inhibition of glial proliferation.

Derivatives of PDMP can also be used in preferred embodiments of the present invention. “PDMP derivatives” can be defined as compounds with structural similarity to PDMP that inhibit the function of glycosylceramide synthase and/or GalT-2. Examples of derivatives of PDMP that may be used with the present invention include D-threo-3′,4′-ethylenedioxy-1-phenyl-2-palmitoylamino-3-pyrrolidino-1-propanol and D-threo-4′-hydroxy-1-phenyl-2-palmitoylamino-3-pyrrolidino-1-propanol (Abe et al., 2001). Other derivatives of PDMP that may be used with the present invention include 1-phenyl-2-hexadecanoylamino-3-morpholino-1-propanol (PPMP). U.S. Pat. Nos. 6,569,889, 5,707,649 and 5,041,441 and U.S. applications US 2002/0198240, US 2003/0073680, and US 2001/0041735 also describe additional PDMP derivatives that may be useful with the present invention.

L-threo PDMP is an optical enantiomer of D-threo PDMP; although structurally similar, these two compounds function very differently. Evidence has suggested that, in contrast to D-threo PDMP, L-threo PDMP can accelerate the biosynthesis of GSL (Inokuchi et al., 1989; U.S. Pat. No. 5,707,649). The LD₅₀ values in mice were higher for L-threo PDMP, as compared do D-threo PDMP (U.S. Pat. No. 5,707,649). Thus, the use of L-threo PDMP is less preferred and could produce deleterious effects in certain embodiments of the present invention.

L-threo PDMP and D-threo PDMP have also shown opposite effects on neurite outgrowth; in primary cultured rat neocortical explants, while D-threo PDMP inhibited both neurite outgrowth and GSL biosynthesis, L-threo PDMP stimulated both neurite outgrowth and GSL biosynthesis (Yamagishi et al., 2003). Several publications have reported that L-threo PDMP can produce beneficial effects, such as improvement in spatial cognition deficits, after ischemia (Yamagishi et al., 2003; Kubota et al., 2000; Inokuchi et al., 1998).

### 2. N-butyldeoxynojirimycin

The imino sugar N-butyldeoxynojirimycin (NB-DNJ) is a potent inhibitor of alpha-glucosidase 1, an enzyme involved in N-glycan synthesis, and an even more potent inhibitor of glucosylceramide glucosyltransferase. U.S. patent application 2003/0069200 describes the use of certain GSL inhibitors including NB-DNJ to treat brain cancer. NB-DNJ may be used with the present invention to treat an inflammatory disease or cytokine disorder.

Derivatives of NB-DNJ may also be used alone, or in combination with the other compounds disclosed in the specification, to treat or prevent inflammatory diseases and conditions. For example, U.S. Pat. No. 6,117,447 describes several NB-DNJ derivatives. Other NB-DNJ derivatives include butyl-deoxygalactonojirimycin.

### 3. 1,5-(butylimino)-1,5-dideoxy-D-glucitol (Miglustat)

1,5-(butylimino)-1,5-dideoxy-D-glucitol (Miglustat) is an inhibitor of glucosylceramide synthase—a glucosyl transferase enzyme that plays a role in the synthesis of many glycosphingolipids. Miglustat is soluble in water. The molecular formula for Miglustat is C₁₀H₂₁NO₄ and has a molecular weight of 219.28. The chemical formula for Miglustat is:

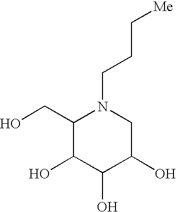

It is contemplated that Miglustat can be used alone, or in combination with the other compounds disclosed in the specification, to treat or prevent inflammatory diseases and conditions.

C. Second Generation Compounds

In addition to the compounds described above, the inventor also contemplates that other sterically similar compounds may be formulated to mimic the key portions of these compounds. Such mimic compounds may be used in the same manner, for example, as an inhibitor of glucosylceramide synthase and/or lactosylceramide synthase.

The generation of further structural equivalents or mimetics may be achieved by the techniques of modeling and chemical design known to those of skill in the art. The art of computer-based chemical modeling is now well known. Using such methods, a chemical compounds acting in a similar manner as an inhibitor of glucosylceramide synthase and/or lactosylceramide synthase can be designed and synthesized. It will be understood that all such sterically similar constructs and second generation molecules fall within the scope of the present invention.

D. GM1 Ganglioside

GM1 is a specific ganglioside; gangliosides are GSL that contain sialic acid. Gangliosides have been reported to be involved in several critical biological functions, including maintenance of membrane integrity and intracellular signal-transmission. Quantitative and qualitative changes in gangliosides are observed during development, aging and disease of the central nervous system (Mendez-Otero and Cavalcante, 2003; Rosner, 2003).

Gangliosides can be found in the central and peripheral nervous systems of mammals. In general, ganglioside concentrations in the gray matter of the brain is higher than in the white and in peripheral nervous tissue. Neurons also usually show higher concentrations of gangliosides than astroglia. Gangliosides are mainly found in the plasma membrane and, in lower concentrations, on the endoplasmic reticulum, the Golgi apparatus, the lysosomes and the nuclear membrane. In the adult brain, the gangliosides GM1, GD1a, GD1b and GT1b account for 80-90% of the total ganglioside content, whereas GD3, a main component of the developing brain, is present only in traces. Certain gangliosides may be useful for the treatment of neurodegenerative disorders such as Alzheimer's and acute brain lesions such as cerebral ischemia (Kracun et al., 1995).

GM1 ganglioside may be used with the present invention to treat inflammatory and/or cytokine-mediated diseases. Preliminary clinical trials have shown that neurodegenerative processes seen with Parkinson's disease, stroke and spinal cord injuries seem to improve by treating patients with GM1 ganglioside (Alter, 1998; Schneider, 1998; Geisler, 1998).

E. Nitric Oxide and Proinflammatory Cytokines

Nitric oxide (NO) is a potent pleiotropic mediator of physiological processes such as smooth muscle relaxation, neuronal signaling, inhibition of platelet aggregation and regulation of cell mediated toxicity. It is a diffusible free radical which plays many roles as an effector molecule in diverse biological systems including neuronal messenger, vasodilation and antimicrobial and antitumor activities (Nathan, 1992; Jaffrey et al., 1995). NO appears to have both neurotoxic and neuroprotective effects and may have a role in the pathogenesis of stroke and other neurodegenerative diseases and in demyelinating conditions (e.g., multiple sclerosis, experimental allergic encephalopathy, X-adrenoleukodystrophy) and in ischemia and traumatic injuries associated with infiltrating macrophages and the production of proinflamatory cytokines (Mitrovic et al., 1994; Bo et al., 1994; Merrill et al., 1993; Dawson et al., 1991, Kopranski et al., 1993; Bonfoco et al., 1995). A number of pro-inflammatory cytokines and endotoxin (bacterial lipopolysaccharide, LPS) also induce the expression of iNOS in a number of cells, including macrophages, vascular smooth muscle cells, epithelial cells, fibroblasts, glial cells, cardiac myocytes as well as vascular and non-vascular smooth muscle cells. Although monocytes/macrophages are the primary source of iNOS in inflammation, LPS and other cytokines induce a similar response in astrocytes and microglia (Hu et al., 1995; Galea et al., 1992).

During inflammation, reactive oxygen species (ROS) are generated by various cells including activated phagocytic leukocytes; for example, during the neutrophil “respiratory burst”, superoxide anion is generated by the membrane-bound NADPH oxidase. ROS are also believed to accumulate when tissues are subjected to inflammatory conditions including ischemia followed by reperfusion. Superoxide is also produced under physiological conditions and is kept in check by superoxide dismutates. Excessively produced superoxide overwhelms the antioxidant capacity of the cell and reacts with NO to form peroxynitrite, ONOO⁻, which may decay and give rise to hydroxyl radicals, OH (Marietta, M., 1989; Moncada et al., 1989; Saran et al., 1990; Beckman et al. 1990). NO, peroxynitrite and OH are potentially toxic molecules to cells including neurons and oligodendrocytes that may mediate toxicity through modification of biomolecules including the formation of iron-NO complexes of iron containing enzyme systems (Drapier et al., 1988), oxidation of protein sulfhydryl groups (Radi et al., 1991), nitration of proteins and nitrosylation of nucleic acids and DNA strand breaks (Wink et al., 1991).

There is now substantial evidence that iNOS plays an important role in the pathogenesis of a variety of diseases. In addition, it is now thought that excess NO production may be involved in a number of conditions, including conditions that involve systemic hypotension such as septic and toxic shock and therapy with certain cytokines. Circulatory shock of various etiologies is associated with profound changes in the body's NO homeostasis. In animal models of endotoxic shock, endotoxin produces an acute release of NO from the constitutive isoform of nitric oxide synthase in the early phase, which is followed by induction of iNOS. NO derived from macrophages, microglia and astrocytes has been implicated in the damage of myelin producing oligodendrocytes in demyelinating disorders like multiple sclerosis and neuronal death during neuronal degenerating conditions including brain trauma (Hu et al., 1995; Galea et al., 1992; Koprowski et al., 1993; Mitrovic et al., 1994; Bo et al., 1994; Merrill et al., 1993).

NO is synthesized from L-arginine by the enzyme nitric oxide synthase (NOS) (Nathan, 1992). Nitric oxide synthases are classified into two groups. One type, constitutively expressed (cNOS) in several cell types (e.g., neurons, endothelial cells), is regulated predominantly at the post-transcriptional level by calmodulin in a calcium dependent manner (Nathan, 1992; Jaffrey et al., 1995). In contrast, the inducible form (iNOS), synthesized de novo in response to different stimuli in various cell types including macrophages, hepatocytes, myocytes, neutrophils, endothelial and messangial cells, is independent of calcium. Astrocytes, the predominant glial component of brain have also been shown to induce iNOS in response to bacterial lipopolysaccharide (LPS) and a series of proinflammatory cytokines including interleukin-1β (IL-1β), tumor necrosis factor-α (TNF-α), interferon-γ (IFN-γ) (Hu et al., 1995; Galea et al., 1992).

Cytokines associated with extracellular signaling are involved in the normal process of host defense against infections and injury, in mechanisms of autoimmunity and in the pathogenesis of chronic inflammatory diseases. It is believed that nitric oxide (NO), synthesized by nitric oxide synthetase (NOS) mediates deleterious effects of the cytokines (Nathan, 1987; Zang et al., 1993; Kubes et al., 1991). For example, NO as a result of stimuli by cytokines (e.g., TNF-α, IL-1 and interleukin-6 (IL-6) is implicated in autoimmune diseases such as multiple sclerosis, rheumatoid arthritis, osteoarthritis (Zang et al., 1993; McCartney-Francis et al., 1993). The NO produced by iNOS is associated with bactericidal properties of macrophages (Nathan, 1992; Stuehr et al., 1989). Recently, an increasing number of cells (including muscle cells, macrophages, keratinocytes, hepatocytes and brain cells) have been shown to induce iNOS in response to a series of proinflammatory cytokines including IL-1, TNF-α, interferon-γ (IFN-γ) and bacterial lipopolysaccharides (LPS) (Zang et al., 1993; Busse et al., 1990; Geng et al., 1995).

F. Inflammatory Diseases

NO generated by iNOS has been implicated in the pathogenesis of inflammatory diseases. In experimental animals hypotension induced by LPS or TNF-alpha can be reversed by NOS inhibitors and reinitiated by L-arginine (Kilbourn et al., 1990). Conditions which lead to cytokine-induced hypotension include septic shock, hemodialysis (Beasley and Brenner, 1992) and IL-2 therapy in cancer patients (Hibbs et al., 1992). Studies in animal models have suggested a role for NO in the pathogenesis of inflammation and pain and NOS inhibitors have been shown to have beneficial effects on some aspects of the inflammation and tissue changes seen in models of inflammatory bowel disease (Miller et al., 1990) and cerebral ischemia and arthritis (Ialenti et al., 1993; Stevanovic-Racic et al., 1994).

Inflammation, iNOS activity and/or cytokine production has been implicated in a variety of diseases and conditions, including psoriasis (Ruzicka et al., 1994; Kolb-Bachofen et al., 1994; Bull et al., 1994); uveitis (Mandia et al., 1994); type 1 diabetes (Eisieik and Leijersfam, 1994; Kroncke et al., 1991; Welsh et al., 1991); septic shock (Petros et al., 1991; Thiemermann & Vane, 1992; Evans et al., 1992; Schilling et al., 1993); pain (Moore et al., 1991; Moore et al, 1992; Meller et al., 1992; Lee et al., 1992); migraine (Olesen et al., 1994); rheumatoid arthritis (Kaurs and Halliwell, 1994); osteoarthritis (Stadler et al., 1991); inflammatory bowel disease (Miller et al., 1993a; Miller et al., 1993b); asthma (Hamid et al., 1993; Kharitonov et al., 1994); Koprowski et al., 1993); immune complex diseases (Mulligan et al., 1992); multiple sclerosis (Koprowski et al., 1993); ischemic brain edema (Nagafuji et al., 1992; Buisson et al., 1992; Trifiletti et al., 1992); toxic shock syndrome (Zembowicz and Vane, 1992); heart failure (Winlaw et al., 1994); ulcerative colitis (Boughton-Smith et al., 1993); atherosclerosis (White et al., 1994); glomerulonephritis (Muhl et al., 1994); Paget's disease and osteoporosis (Lowick et al., 1994); inflammatory sequelae of viral infections (Koprowski et al., 1993); retinitis, (Goureau et al., 1992); oxidant induced lung injury (Berisha et al., 1994); eczema (Ruzica et al., 1994); acute allograft rejection (Devlin, J. et al., 1994); and infection caused by invasive microorganisms which produce NO (Chen and Rosazza, 1994).

In the central nervous system, apoptosis may play an important pathogenetic role in neurodegenerative diseases such as iscehmic injury and white matter diseases (Thompson, 1995; Bredesen, 1995). Both X-linked adrenoleukodystrophy (X-ALD) and multiple sclerosis (MS) are demyelinating diseases with the involvement of proinflammatory cytokines in the manifestation of white matter inflammation. The presence of immunoreactive tumor necrosis factor a (TNF-α) and interleukin 1 (IL-1β) in astrocytes and microglia of X-ALD brain has indicated the involvement of these cytokines in immunopathology of X-ALD and aligned X-ALD with MS, the most common immune-mediated demyelinating disease of the CNS in man (Powers, 1995; Powers et al., 1992; McGuinnes et al., 1995; McGuiness et al., 1997). Several studies demonstrating the induction of proinflammatory cytokines at the protein or mRNA level in cerebrospinal fluid and brain tissue of MS patients have established an association of proinflammatory cytokines (TNF-α, IL-1β, IL-2, IL-6, and IFN-γ) with the inflammatory loci in MS (Maimone et al., 1991; Tsukada et al., 1991; Rudick and Ransohoff, 1992).

X-linked adrenoleukodystrophy (X-ALD), an inherited, recessive peroxisomal disorder, is characterized by progressive demyelination and adrenal insufficiency (Singh, 1997; Moser et al., 1984). It is the most common peroxisomal disorder affecting between 1/15,000 to 1/20,000 boys and manifests with different degrees of neurological disability. The onset of childhood X-ALD, the major form of X-ALD, is between the age of 4 to 8 and then death within the next 2 to 3 years. Although X-ALD presents as various clinical phenotypes, including childhood X-ALD, adrenomyeloneuropathy (AMN), and Addison's disease, all forms of X-ALD are associated with the pathognomonic accumulation of saturated very long chain fatty acids (VLCFA) (those with more than 22 carbon atoms) as a constituent of cholesterol esters, phospholipids and gangliosides (Moser et al., 1984) and secondary neuroinflammatory damage (Moser et al., 1995). The necrologic damage in X-linked adrenoleukodystrophy may be mediated by the activation of astrocytes and the induction of proinflammatory cytokines. Due to the presence of similar concentration of VLCFA in plasma and as well as in fibroblasts of X-ALD, fibroblasts are generally used for both prenatal and postnatal diagnosis of the disease (Singh, 1997; Moser et al., 1984).

The deficient activity for oxidation of lignoceroyl-CoA ligase as compared to the normal oxidation of lignoceroyl-CoA in purified peroxisomes isolated from fibroblasts of X-ALD indicated that the abnormality in the oxidation of VLCFA may be due to deficient activity of lignoceroyl-CoA ligase required for the activation of lignoceric acid to lignoceroyl-CoA (Hashmi et al., 1986; Lazo et al., 1988). While these metabolic studies indicated lignoceroyl-CoA ligase gene as a X-ALD gene, positional cloning studies led to the identification of a gene that encodes a protein (ALDP), with significant homology with the ATP-binding cassette (ABC) of the super-family of transporters (Mosser et al., 1993). The normalization of fatty acids in X-ALD cells following transfection of the X-ALD gene (Cartier et al., 1995) supports a role for ALDP in fatty acid metabolism; however, the precise function of ALDP in the metabolism of VLCFA is not known at present.

Similar to other genetic diseases affecting the central nervous system, the gene therapy in X-ALD does not seem to be a real option in the near future and in the absence of such a treatment a number of therapeutic applications have been investigated (Singh, 1997; Moser, 1995). Adrenal insufficiency associated with X-ALD responds readily with steroid replacement therapy, however, there is as yet no proven therapy for neurological disability (Moser, 1995). Addition of monoenoic fatty acid (e.g., oleic acid) to cultured skin fibroblasts of X-ALD patients causes a reduction of saturated VLCFA presumably by competition for the same chain elongation enzyme (Moser, 1995). Treatment of X-ALD patients with trioleate resulted in 50% reduction of VLCFA. Subsequent treatment of X-ALD patients with a mixture of trioleate and trieruciate (popularly known as Lorenzo's oil) also led to a decrease in plasma levels of VLCFA (Moser, 1995; Rizzo et al., 1986; Rizzo et al., 1989). Unfortunately, the clinical efficacy has been unsatisfactory since no proof of favorable effects has been observed by attenuation of the myelinolytic inflammation in X-ALD patients (Moser, 1995). Moreover, the exogenous addition of unsaturated VLCFA induces the production of superoxide, a highly reactive oxygen radical, by human neutrophils (Hardy et al., 1994). Since cerebral demyelination of X-ALD is associated with a large infiltration of phagocytic cells to the site of the lesion (Powers et al., 1992), treatment with unsaturated fatty acids may even be toxic to X-ALD patients. Bone marrow therapy also appears to be of only limited value because of the complexicity of the protocol and of insignificant efficacy in improving the clinical status of the patient (Moser, 1995).

Experimental allergic encephalomyelitis (EAE) is an inflammatory demyelinating disease of the central nervous system (CNS) that serves as a model for the human demyelinating disease, multiple sclerosis (MS). Studies have shown that the majority of the inflammatory cells constitute of T-lymphocytes and macrophages (Merrill and Benveniste, 1996). These effector cells and astrocytes have been implicated in the disease pathogenesis by secreting number of molecules that act as inflammatory mediators and/or tissue damaging agents such as nitric oxide (NO). NO is a molecule with beneficial as well as detrimental effects. In neuroinflammatory diseases like EAE, high amounts of NO produced for longer durations by inducible nitric oxide synthase (iNOS) acts as a cytotoxic agent towards neuronal cells. Previous studies have shown NO by itself or it's reactive product (ONOO⁻) may be responsible for death of oligodendrocytes, the myelin producing cells of the CNS, and resulting in demyelination in the neuroinflammatory disease processes (Merrill et al., 1993; Mitrovic et al., 1994).

Infiltrating T-lymphocytes in EAE produce pro-inflammatory cytokines such as IL-12, TNF-α and IFN-γ (Merrill and Benveniste, 1996). In addition to T-cells and macrophages, astrocytes have also been shown to produce TNF-α (Shafer and Murphy, 1997). Convincing evidence exists to support a role for both TNF-α and IFN-γ in the pathogenesis of EAE (Taupin et al., 1997; Villarroya et al., 1996; Issazadeh et al., 1995). Investigations with antibodies against TNF-α have shown that in mice these antibodies protect against active and adaptively transferred EAE disease (Klinkert et al., 1997). The expression of TNF-α and IFN-γ during EAE disease could result in the upregulation of iNOS in macrophage and astrocytes because TNF-α and IFN-γ have been shown to be potent inducers of iNOS in macrophages and astrocytes in culture (Xie et al., 1994). This induction of iNOS could result in the production of NO, which if produced in large amounts may lead to cytotoxic effects. Peroxynitrite (ONOO⁻) has been identified in both MS and EAE CNS (Hooper et al., 1997; van der Veen et al., 1997). The role of peroxynitrite in the pathogenesis of EAE is supported by the beneficial effects of uric acid, a peroxynitrite scavenger, against EAE and by a subsequent survey documenting that MS patients had significantly lower serum uric acid levels than those of controls (Hooper et al., 1998). However, aggravation of EAE by inhibitors of NOS activity (Ruuls et al., 1996) and in an animal model of iNOS gene knockout (Fenyk-Melody et al., 1998) indicate that NO may not be the only pathological mediator in EAE disease process. In addition to NO other free radicals such as reactive oxygen intermediates (O₂⁻, H₂O₂, and OH⁻) can also be stimulated by cytokines (Merrill and Benveniste, 1996). Reactive oxygen intermediates (ROI) and NO are believed to be key mediators of pathophysiological changes that take place during inflammatory disease process. ROI's such as superoxide anion, hydroxy radicals and hydrogen peroxide can also be stimulated by TNF-α (Merrill and Benveniste, 1996). Therefore, it is likely that both the direct modulation of cellular functions by proinflammatory cytokines and toxicity of the ROI and reactive nitrogen species may play a role in the pathogenesis of EAE disease.

Several studies on protein and/or mRNA levels in plasma, cerebrospinal fluid (CSF), brain tissue, and cultured blood leukocytes from MS patients have established an association of proinflammatory cytokines (TNF-α, IL-1 and IFN-γ) with MS (Taupin et al., 1997; Villarroya et al., 1996; Issazadeh et al., 1995). The mRNA for iNOS has also been detectable in both MS as well as EAE brains (Bagasra et al., 1995; Koprowski et al., 1993). Semiquantitative RT-PCR™ for iNOS mRNA in MS brains shows markedly higher expression of iNOS mRNA in MS brains than control brains (Bagasra et al., 1995). Analysis of CSF from MS patients has also shown increased levels of nitrite and nitrate compared with normal control (Merrill and Benveniste, 1996). Peroxynitrite, ONOO— is a strong nitrosating agent capable of nitrosating tyrosine residues of proteins to nitrotyrosine. Increased levels of nitrotyrosine have been found in demyelinating lesions of MS brains as well as spinal cords of mice with EAE (Hooper et al., 1998; Hooper et al., 1997). A strong correlation exists between CSF levels of cytokines, disruption of blood-brain barrier, and high levels of circulating cytokines in MS patients (Villarroya et al., 1996; Issazadeh et al., 1995). Increase in TNF-α and IFN-γ levels seems to predict relapse in MS and the number of circulating IFN-γ positive blood cells correlates with severity of disability. These observations support the view that in both MS and EAE, induction of proinflammatory cytokines and production of NO through iNOS play roles in the pathogenesis of these diseases.

Alzheimer 's disease (AD) is the most common degenerative dementia affecting primarily the elderly population. The disease is characterized by the decline of multiple cognitive functions and a progressive loss of neurons in the central nervous system. Deposition of beta-amyloid peptide has also been associated with AD. Over the last decade, a number of investigators have noted that AD brains contain many of the classical markers of immune mediated damage. These include elevated numbers of microglia cells, which are believed to be an endogenous CNS form of the peripheral macrophage, and astrocytes. Of particular importance, complement proteins have been immunohistochemically detected in the AD brain and they most often appear associated with beta-amyloid containing pathological structures known as senile plaques (Rogers et al., 1992; Haga et al., 1993).

These initial observations which suggest the existence of an inflammatory component in the neurodegeneration observed in AD has been extended to the clinic. A small clinical study using the nonsteroidal anti-inflammatory drug, indomethacin, indicated that indomethacin significantly slowed the progression of the disease (Rogers et al., 1993). In addition, a study examining age of onset among 50 elderly twin pairs with onsets of AD separated by three or more years, suggested that anti-inflammatory drugs may prevent or delay the initial onset of AD symptoms (Breitner et al., 1994).

Over the years numerous therapies have been tested for the possible beneficial effects against EAE or MS disease but with mixed results (Cross et al., 1994; Ruuls et al., 1996). Though aminoguandine (AG) has been described as a competitive inhibitor of iNOS and a suppressor of its expression (Corbett and McDaniel, 1996; Joshi et al., 1996), to date few compounds which inhibit iNOS are of potential therapeutic value have been identified. This deficiency is particularly troubling given the significant cellular damage which can arise as a result of iNOS-mediated nitric oxide toxicity, especially in chronic inflammatory disease states. There is a present need for therapeutic agents which will inhibit or even prevent cytotoxic concentrations of NO from occurring in individuals suffering from diseases and conditions to which NO toxicity or an undesired production of proinflammatory cytokines is linked.

G. Optimization in Therapy

A compound identified as having the ability to treat or prevent an inflammatory disease in a subject can be assayed by its optimum therapeutic dosage alone or in combination with another such compound. Such assays are well known to those of skill in the art, and include tissue culture or animal models for various disorders that are treatable with such agents.

Examples of such assays include those described herein and in U.S. Pat. No. 5,696,109. For instance, an assay to determine the therapeutic potential of molecules in brain ischemia (stroke) evaluates an agent's ability to prevent irreversible damage induced by an anoxic episode in brain slices maintained under physiological conditions. An animal model of Parkinson's disease involving iatrogenic hydroxyl radical generation by the neurotoxin MPTP (Chiueh et al., 1992, incorporated herein by reference) may be used to evaluate the protective effects of iNOS or pro-inflammatory cytokine induction inhibitors. The neurotoxin, MPTP, has been shown to lead to the degeneration of dopaminergic neurons in the brain, thus providing a good model of experimentally induced Parkinson's disease (e.g., iatrogenic toxicity). An animal model of ischemia and reperfusion damage is described using isolated iron-overloaded rat hearts to measure the protective or therapeutic benefits of an agent. Briefly, rats receive an intramuscular injection of an iron-dextran solution to achieve a significant iron overload in cardiac tissue. Heart are then isolated and then subjected to total global normothermic ischemia, followed by reperfusion with the perfusion medium used initially. During this reperfusion, heart rate, and diastolic and systolic pressures were monitored. Cardiac tissue samples undergo the electron microscopy evaluation to measure damage to mitochondria such as swelling and membrane rupture, and cell necrosis. Comparison of measured cardiac function and cellular structural damage with or without the agent or iron-overloading after ischemia/reoxygenation is used to determine the therapeutic effectiveness of the agent.

H. Pharmaceutical Compositions

Pharmaceutical compositions of the present invention comprise an effective amount of one or more glycosphingolipid inhibitor, preferably a glucosylceramide synthase and/or GalT-2 inhibitor, or additional agent dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of an pharmaceutical composition that contains at least one glycosphingolipid inhibitor, preferably a glucosylceramide synthase and/or GalT-2 inhibitor, or additional active ingredient will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the therapeutic or pharmaceutical compositions is contemplated.

The glycosphingolipid inhibitor, preferably a glucosylceramide synthase and/or GalT-2 inhibitor, may comprise different types of carriers depending on whether it is to be administered in solid, liquid or aerosol form, and whether it need to be sterile for such routes of administration as injection. The present invention can be administered intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, intraperitoneally, subcutaneously, subconjunctival, intravesicularlly, mucosally, intrapericardially, intraumbilically, intraocularally, orally, topically, locally, inhalation (e.g. aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

The actual dosage amount of a composition of the present invention administered to an animal patient can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

In any case, the composition may comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

The glycosphingolipid inhibitor, preferably a glucosylceramide synthase and/or GalT-2 inhibitor, may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine.

In embodiments where the composition is in a liquid form, a carrier can be a solvent or dispersion medium comprising but not limited to, water, ethanol, polyol (e.g., glycerol, propylene glycol, liquid polyethylene glycol, etc), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. In many cases, it will be preferable to include isotonic agents, such as, for example, sugars, sodium chloride or combinations thereof.

In other embodiments, one may use eye drops, nasal solutions or sprays, aerosols or inhalants in the present invention. Such compositions are generally designed to be compatible with the target tissue type. In a non-limiting example, nasal solutions are usually aqueous solutions designed to be administered to the nasal passages in drops or sprays. Nasal solutions are prepared so that they are similar in many respects to nasal secretions, so that normal ciliary action is maintained. Thus, in preferred embodiments the aqueous nasal solutions usually are isotonic or slightly buffered to maintain a pH of about 5.5 to about 6.5. In addition, antimicrobial preservatives, similar to those used in ophthalmic preparations, drugs, or appropriate drug stabilizers, if required, may be included in the formulation. For example, various commercial nasal preparations are known and include drugs such as antibiotics or antihistamines.

In certain embodiments the glycosphingolipid inhibitor, preferably a glucosylceramide synthase and/or GalT-2 inhibitor, is prepared for administration by such routes as oral ingestion. In these embodiments, the solid composition may comprise, for example, solutions, suspensions, emulsions, tablets, pills, capsules (e.g., hard or soft shelled gelatin capsules), sustained release formulations, buccal compositions, troches, elixirs, suspensions, syrups, wafers, or combinations thereof. Oral compositions may be incorporated directly with the food of the diet. Preferred carriers for oral administration comprise inert diluents, assimilable edible carriers or combinations thereof. In other aspects of the invention, the oral composition may be prepared as a syrup or elixir. A syrup or elixir, and may comprise, for example, at least one active agent, a sweetening agent, a preservative, a flavoring agent, a dye, a preservative, or combinations thereof.

In certain preferred embodiments an oral composition may comprise one or more binders, excipients, disintegration agents, lubricants, flavoring agents, and combinations thereof. In certain embodiments, a composition may comprise one or more of the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc.; or combinations thereof the foregoing. When the dosage unit form is a capsule, it may contain, in addition to materials of the above type, carriers such as a liquid carrier. Various other materials may be present as coatings or to otherwise modify the physical form of the dosage unit. For instance, tablets, pills, or capsules may be coated with shellac, sugar or both.

Additional formulations which are suitable for other modes of administration include suppositories. Suppositories are solid dosage forms of various weights and shapes, usually medicated, for insertion into the rectum, vagina or urethra. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The preparation of highly concentrated compositions for direct injection is also contemplated, where the use of DMSO as solvent is envisioned to result in extremely rapid penetration, delivering high concentrations of the active agents to a small area.

The composition must be stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein.

In particular embodiments, prolonged absorption of an injectable composition can be brought about by the use in the compositions of agents delaying absorption, such as, for example, aluminum monostearate, gelatin or combinations thereof.

I. Screening for Modulators of the Protein Function

The present invention further comprises methods for identifying modulators of the function of glucosylceramide synthase and/or GalT-2. These assays may comprise random screening of large libraries of candidate substances; alternatively, the assays may be used to focus on particular classes of compounds selected with an eye towards structural attributes that are believed to make them more likely to modulate the function of glucosylceramide synthase and/or GalT-2.

By function, it is meant that one may assay for the production of GluCer and/or LacCer.

To identify a glucosylceramide synthase and/or GalT-2 modulator, one generally will determine the function of glucosylceramide synthase and/or GalT-2 in the presence and absence of the candidate substance, a modulator defined as any substance that alters function. For example, a method generally comprises:
(a) providing a candidate modulator;
(b) admixing the candidate modulator with an isolated compound or cell, or a suitable experimental animal;
(c) measuring one or more characteristics of the compound, cell or animal in step (c); and
(d) comparing the characteristic measured in step (c) with the characteristic of the compound, cell or animal in the absence of said candidate modulator,
wherein a difference between the measured characteristics indicates that said candidate modulator is, indeed, a modulator of the compound, cell or animal.
   Assays may be conducted in cell free systems, in isolated cells, or in organisms including transgenic animals.

It will, of course, be understood that all the screening methods of the present invention are useful in themselves notwithstanding the fact that effective candidates may not be found. The invention provides methods for screening for such candidates, not solely methods of finding them.

1. Modulators

As used herein the term “candidate substance” refers to any molecule that may potentially inhibit or enhance glucosylceramide synthase and/or GalT-2 activity. The candidate substance may be a protein or fragment thereof, a small molecule, or even a nucleic acid molecule. It may prove to be the case that the most useful pharmacological compounds will be compounds that are structurally related to PDMP. Using lead compounds to help develop improved compounds is know as “rational drug design” and includes not only comparisons with know inhibitors and activators, but predictions relating to the structure of target molecules.

The goal of rational drug design is to produce structural analogs of biologically active polypeptides or target compounds. By creating such analogs, it is possible to fashion drugs, which are more active or stable than the natural molecules, which have different susceptibility to alteration or which may affect the function of various other molecules. In one approach, one would generate a three-dimensional structure for a target molecule, or a fragment thereof. This could be accomplished by x-ray crystallography, computer modeling or by a combination of both approaches.

It also is possible to use antibodies to ascertain the structure of a target compound activator or inhibitor. In principle, this approach yields a pharmacore upon which subsequent drug design can be based. It is possible to bypass protein crystallography altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-idiotypes may be generated using the methods described herein for producing antibodies, using an antibody as the antigen.

On the other hand, one may simply acquire, from various commercial sources, small molecule libraries that are believed to meet the basic criteria for useful drugs in an effort to “brute force” the identification of useful compounds. Screening of such libraries, including combinatorially generated libraries (e.g., peptide libraries), is a rapid and efficient way to screen large number of related (and unrelated) compounds for activity. Combinatorial approaches also lend themselves to rapid evolution of potential drugs by the creation of second, third and fourth generation compounds modeled of active, but otherwise undesirable compounds.

Candidate compounds may include fragments or parts of naturally-occurring compounds, or may be found as active combinations of known compounds, which are otherwise inactive. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds. Thus, it is understood that the candidate substance identified by the present invention may be peptide, polypeptide, polynucleotide, small molecule inhibitors or any other compounds that may be designed through rational drug design starting from known inhibitors or stimulators.

Other suitable modulators include antisense molecules, ribozymes, and antibodies (including single chain antibodies), each of which would be specific for the target molecule. Such compounds are described in greater detail elsewhere in this document. For example, an antisense molecule that bound to a translational or transcriptional start site, or splice junctions, would be ideal candidate inhibitors.

In addition to the modulating compounds initially identified, the inventors also contemplate that other sterically similar compounds may be formulated to mimic the key portions of the structure of the modulators. Such compounds, which may include peptidomimetics of peptide modulators, may be used in the same manner as the initial modulators.

An inhibitor according to the present invention may be one which exerts its inhibitory or activating effect upstream, downstream or directly on glucosylceramide synthase and/or GalT-2. Regardless of the type of inhibitor or activator identified by the present screening methods, the effect of the inhibition or activator by such a compound results in decreases in the production of GluCer and/or LacCer as compared to that observed in the absence of the added candidate substance.

2. In Vitro Assays

A quick, inexpensive and easy assay to run is an in vitro assay. Such assays generally use isolated molecules, can be run quickly and in large numbers, thereby increasing the amount of information obtainable in a short period of time. A variety of vessels may be used to run the assays, including test tubes, plates, dishes and other surfaces such as dipsticks or beads.

One example of a cell free assay is a binding assay. While not directly addressing function, the ability of a modulator to bind to a target molecule in a specific fashion is strong evidence of a related biological effect. For example, binding of a molecule to a target may, in and of itself, be inhibitory, due to steric, allosteric or charge-charge interactions. The target may be either free in solution, fixed to a support, expressed in or on the surface of a cell. Either the target or the compound may be labeled, thereby permitting determining of binding. Usually, the target will be the labeled species, decreasing the chance that the labeling will interfere with or enhance binding. Competitive binding formats can be performed in which one of the agents is labeled, and one may measure the amount of free label versus bound label to determine the effect on binding.

A technique for high throughput screening of compounds is described in WO 84/03564. Large numbers of small peptide test compounds are synthesized on a solid substrate, such as plastic pins or some other surface. Bound polypeptide is detected by various methods.

3. In Cyto Assays

The present invention also contemplates the screening of compounds for their ability to modulate glucosylceramide synthase and/or GalT-2 in cells. Various cell lines can be utilized for such screening assays, including cells specifically engineered for this purpose.

Depending on the assay, culture may be required. The cell is examined using any of a number of different physiologic assays. Alternatively, molecular analysis may be performed, for example, looking at protein expression, mRNA expression (including differential display of whole cell or polyA RNA) and others.

4. In Vivo Assays

In vivo assays involve the use of various animal models, including transgenic animals that have been engineered to have specific defects, or carry markers that can be used to measure the ability of a candidate substance to reach and effect different cells within the organism. Due to their size, ease of handling, and information on their physiology and genetic make-up, mice are a preferred embodiment, especially for transgenics. However, other animals are suitable as well, including rats, rabbits, hamsters, guinea pigs, gerbils, woodchucks, cats, dogs, sheep, goats, pigs, cows, horses and monkeys (including chimps, gibbons and baboons). Assays for modulators may be conducted using an animal model derived from any of these species.

In such assays, one or more candidate substances are administered to an animal, and the ability of the candidate substance(s) to alter one or more characteristics, as compared to a similar animal not treated with the candidate substance(s), identifies a modulator. The characteristics may be any of those discussed above with regard to the function of a particular compound (e.g., enzyme, receptor, hormone) or cell (e.g., growth, tumorigenicity, survival), or instead a broader indication such as behavior, anemia, immune response, etc.

The present invention provides methods of screening for a candidate substance that inhibits glucosylceramide synthase and/or GalT-2. In these embodiments, the present invention is directed to a method for determining the ability of a candidate substance to inhibit the production of GluCer and/or LacCer, generally including the steps of: administering a candidate substance to the animal; and determining the ability of the candidate substance to reduce one or more characteristics of inhibiting the production of GluCer and/or LacCer, preferably resulting in the reduction of inflammatory and/or cytokine mediated responses.

Treatment of these animals with test compounds will involve the administration of the compound, in an appropriate form, to the animal. Administration will be by any route that could be utilized for clinical or non-clinical purposes, including but not limited to oral, nasal, buccal, or even topical. Alternatively, administration may be by intratracheal instillation, bronchial instillation, intradermal, subcutaneous, intramuscular, intraperitoneal or intravenous injection. Specifically contemplated routes are systemic intravenous injection, regional administration via blood or lymph supply, or directly to an affected site.

Determining the effectiveness of a compound in vivo may involve a variety of different criteria. Also, measuring toxicity and dose response can be performed in animals in a more meaningful fashion than in in vitro or in cyto assays.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

### Materials and Methods

### Reagents.

Recombinant rat interferon gamma (IFNγ) and recombinant rat tumor necrosis factor-alpha (TNFα) was obtained from Calbiochem (CA). N-Acetyl cysteine (NAC), pyrrolidine dithiocarbamate (PDTC) and Lipopolysaccharide, (from Escherichia coli Serotype 0111:B4) was from Sigma (MO). DMEM and FBS were from Life Technologies Inc. Glucosylceramide (GluCer), lactosylceramide (LacCer), galactosylceramide (GalCer), gangliosides, N,N-Dimethylsphingosine, sphingosine-1-phosphate, and D-threo-1-Phenyl-2-decanoylamino-3-morpholino-1-propanol.HCI (PDMP) were from Matreya Inc (PA). [¹⁴C]Galactose and [³H]UDP-Galactose were obtained from American Radiolabeled Chemicals (MO). 2-(4-Morpholinyl)-8-phenyl-4H-1-benzopyran-4-one (LY294002) and PD98059 were obtained from BIOMOL research Laboratories (PA). PI, phosphatidylserine, and lipid standards were purchased from Matreya (U.S.A.). [γ-32P] ATP (3,000 Ci/mmol) was from Amersham Pharmacia Biotech (U.S.A.).

### Cell Culture.

Primary astrocyte-enriched cultures were prepared from the whole cortex of one day old Sprague-Dawley rats as described earlier (Pahan et al., 1998b). Briefly, the cortex was rapidly dissected in ice-cold calcium/magnesium free Hanks Balanced Salt Solution (HBSS) (Gibco, Grand Island, N.Y.) at pH 7.4 as described previously (Won et al., 2001). The tissue was then minced, incubated in HBSS containing trypsin (2 mg/ml) for 20 min and washed twice in plating medium containing 10% FBS and 10 μg/ml gentamicin, and then disrupted by triturating through a Pasteur pipette following which cells were seeded in 75-cm² culture flasks (Falcon, Franklin, N.J.). After incubation at 37° C. in 5% CO₂ for 1 day, the medium was completely changed to the culture medium (DMEM containing 5% FBS and 10 μg/ml gentamicin). The cultures received half exchanges with fresh medium twice a week. After 14-15 days the cells were shaken for at least 24 h on an orbital shaker to remove the microglia and then seeded on multi-well tissue culture dishes. The cells were incubated with serum-free DMEM for 24 h prior to the incubation with drugs.

C6 rat glioma cells obtained from ATCC were maintained in Dulbecco's modified Eagle's medium (DMEM) (GIBCO, CA) containing 10% fetal bovine serum (FBS) (GIBCO) and 10 μg/ml gentamicin. All the cultured cells were maintained at 37° C. in 5% CO₂. At 80% confluency, the cells were incubated with serum free DMEM medium for 24 h prior to the incubation with LPS/IFNγ and other chemicals.

### BrdU Incorporation Assay.

Proliferation of primary astrocytes was assayed by using the Cell proliferation ELISA, BrdU calorimetric assay kit (Roche, Germany) according to manufacturer's protocol. Briefly, cells were seeded in 96 well plates in quadruplicate and following overnight serum starvation were stimulated with mitogenic stimulants. 2 h before termination of proliferation assay, BrdU (10 μM) was added to each well following which cells were fixed and levels of incorporated BrdU were assayed by using a conjugated anti-BrdU enzyme. Colorimetric analysis was done by measuring absorbance at 370 nm using a spectramax MAX 190 (Molecular devices) multi-well plate reading spectrophotometer.

### Assay for NO Production.

Cells were cultured in 12-well plastic tissue culture plates. Following appropriate treatment, production of NO was determined by an assay of the culture supernatant for nitrite (Green et al., 1982). Briefly, 100 μl of culture supernatant was allowed to react with 100 μl of Griess reagent. The optical density of the assay samples was measured spectrophotometrically at 570 nm. Nitrite concentrations were calculated from a standard curve derived from the reaction of NaNO₂ in fresh media.

### Western Blot Analysis.

For iNOS protein, the cells were washed with cold Tris buffered saline (TBS; 20 mM Trizma base, and 137 mM NaCl, pH 7.5), lysed in 1×SDS sample loading buffer (62.5 mM Trizma base, 2% w/v SDS, 10% glycerol), following sonication and centrifugation at 15,000×g for 5 min, the supernatant was used for the iNOS western immunoblot assay. The protein concentration of samples was determined with the detergent compatible protein assay reagent (Bio-Rad Laboratories, CA) using bovine serum albumin (BSA) as the standard. Sample was boiled for 3 min with 0.1 volumes of 10% β-mercaptoethanol and 0.5% bromophenol blue mix. Fifty μg of total cellular protein was resolved by electrophoresis on 8 or 12% polyacrylamide gels, electro-transferred to polyvinylidene difluoride (PVDF) filter and blocked with Tween 20 containing Tris-buffered saline (TBST; 10 mM Trizma base-pH 7.4, 1% Tween 20, and 150 mM NaCl) with 5% skim milk. After incubation with antiserum against iNOS (BD PharMingen, CA), rat GalT-2 (Abjent Inc., CA), H-Ras (Upstate Biotechnology, CA), GFAP (Santa Cruz Biotech, CA), or phospho-specific ERK1/2 (Cell signaling Tech Inc., MA), or pIκ-B (Signal Transduction), in PVDF buffer for 2 h at room temperature, the filters were washed 3 times with TBST buffer and then incubated with horseradish peroxidase conjugated anti-rabbit or mouse IgG for 1 h. The membranes were detected by autoradiography using ECL-plus (Amersham Pharmacia Biotech) after washing with TBST buffer.

### Nuclear Extraction and Electrophoretic Mobility Shift Assay.

Nuclear extracts from cells (1×10⁷ cells) were prepared using a previously published method (Dignam et al., 1983) with slight modifications. Cells were harvested, washed twice with ice-cold TBS, and lysed in 400 μl of buffer A containing 10 mM KCl, 2 mM MgCl₂, 0.5 mM dithiothreitol, protease inhibitor cocktail (Sigma), and 0.1% Nonidet P-40 in 10 mM HEPES, pH 7.9 for 10 min on ice. Following centrifugation at 5,000×g for 10 min, the pelleted nuclei were washed with buffer A without Nonidet P-40, and resuspended in 40 μl of buffer B containing 25% (v/v) glycerol, 0.42M NaCl, 1.5 mM MgCl₂, 0.2 mM EDTA, 0.5 mM dithiothreitol, and Complete™ protease inhibitor cocktail (Roche) in 20 mM HEPES, pH 7.9 for 30 min on ice. The lysates were centrifuged at 15,000×g for 15 min and the supernatants containing the nuclear proteins were stored at −70° C. until use. Ten μg of nuclear proteins was used for the electrophoretic mobility shift assay for detection of NF-κB DNA binding activities. DNA-protein binding reactions were carried out at room temperature for 20 min in 10 mM Trizma base (pH 7.9), 50 mM NaCl, 5 mM MgCl₂, 1 mM EDTA, 1 mM dithiothreitol, 1 μg poly (dI-dC), 5% (v/v) glycerol, and approximately 0.3 pmol of NF-κB probe (Santa Cruz Biotech) labeled with DIG-ddUTP using terminal deoxynucleotidyl transferase (Roche). Protein-DNA complexes were resolved from protein-free DNA in 5% polyacrylamide gels at room temperature in 50 mM Tris, pH 8.3, 0.38M glycine, and 2 mM EDTA, and electroblotted onto positively charged nylon membranes. The chemiluminescent autoradiography detection was performed as suggested by the manufacturer (Roche Molecular Biochemicals), using an alkaline phosphatase conjugated anti-DIG F_{ab} fragment (Roche Molecular Biochemicals) and CSPD (Roche Molecular Biochemicals).

### Plamids and Transient Transfections and Reporter Gene Assay.

Dominant negative and constitutively active ras expression vector (pCMVrasN17 and pCMVrasv12) and KB repeat luciferase reporter construct (pNF-κB-Luc) were purchased from BD Biosciences. 3×10⁵ cells/well were cultured in 6-well plates for one day before the transfection. Transfection was performed with plasmid concentration constant (2.5 μg/transfection) and 8 μl of Fugene transfection reagent (Roche Molecular Biochemicals). 24 h after transfection, the cells were placed in serum free media for overnight. Following appropriate treatment, the cells were washed with phosphate buffered saline (PBS), scraped, and then resuspended with 100 μl of lysis buffer (40 mM of Tricine pH 7.8, 50 mM of NaCl, 2 mM of EDTA, 1 mM of MgSO₄, 5 mM of dithiothreitol, and 1% of Triton X-100). After incubation at room temperature for 15 min with occasional vortexing, the samples were centrifuged. The luciferase and β-galactosidase activities were measured by using luciferase assay kit (Stratagene, CA) and β-gal assay kit (Invitrogen, CA) respectively. The emitted light and optical absorbance was measured using Spectra Max/Gemini XG (Molecular Device, CA) and SpectraMax 190 (Molecular Device).

### Quantification of Ras Activation.

After stimulation, primary astrocytes in 6-well plates were washed with ice cold PBS and lysed in membrane lysis buffer (MLB; 0.5 ml of 25 mM HEPES, pH 7.5, 150 mM NaCl, 1% Igepal CA-630, 0.25% sodium deoxycholate, 10% glycerol, 10 mM MgCl₂, 1 mM EDTA, 25 mM NaF, 1 mM of sodium orthovanadate, and EDTA free Complete™ protease inhibitor cocktail). After centrifugation (5,000×g) at 4° C. for 5 min, supernatant was used for Ras activation assay. One hundred μg of supernatant was used for binding with agarose conjugated Ras-binding domain (RBD) of Raf-1 which was expressed in BL21 (Invitrogen), Escherichia coli strain, transformed by pGEX-2T-GST-RBD in the presence of 0.1 mM of IPTG as described previously (Herrmann et al., 1995). The binding reaction was performed at 4° C. for 30 min in MLB. Following washing with MLB three times, Ras-RBD complex were denatured by adding of 2×SDS sample buffer. Ras protein was identified by western blot analysis with Ras antibodies from Upstate Biotechnology.

### PI-3 Kinase Activity Assay.

Cells after stimulation in serum-free DMEM/F-12 were lysed with ice-cold lysis buffer containing 1% (vol/vol) NP-40,100 mM NaCl, 20 mM Tris (pH 7.4), 10 mM iodoacetamide, 10 mM NaF, 1 mM sodium orthovanadate, 1 mM phenylmethylsulfonylchloride, 1 μg/ml leupeptin, 1 μg/ml antipain, 1 mg/ml aprotinin, and 1 μg/ml pepstatin A. Lysates were incubated at 4° C. for 15 min, followed by centrifugation at 13,000 g for 15 min. The supernatant was pre-cleared with protein G-Sepharose beads (Pharmacia Biotech) for 1 h at 4° C. followed by the addition of 1 μg/ml p85α monoclonal antibody. After 2 h of incubation at 4° C., protein G-Sepharose beads were added and the resulting mixture was further incubated for 1 h at 4° C. The immunoprecipitates were washed twice with lysis buffer, once with phosphate-buffered saline (PBS), once with 0.5 M LiCl and 100 mM Tris (pH 7.6), once in water, and once in kinase buffer (5 mM MgCl2, 0.25 mM EDTA, 20 mM HEPES, pH 7.4). PI-3kinase activity was determined as already described (Ward et al., 1992; Pahan et al., 1999) using a lipid mixture of 100 μl of 0.1 mg/ml PI and 0.1 mg/ml phosphatidylserine dispersed by sonication in 20 mM HEPES (pH 7.0) and 1 mM EDTA. The reaction was initiated by the addition of 20 μCi of [γ-32P] ATP (3,000 Ci/mmol; DuPont NEN) and 100 μM ATP and terminated after 15 min by the addition of 80 μl of 1 M HCl and 200 μl of chloroform/methanol (1:1). Phospholipids were separated by TLC and visualized by exposure to iodine vapor and autoradiography.

### Measurement of LacCer Synthesis.

Cultured cells were incubated in growth medium containing [¹⁴C]galactose (5 μCi/ml) for 24 h as described previously. The medium was removed, and the cell monolayer was washed with sterile PBS. After the stimulation with LPS/IFNγ (1 μg/ml; 10 U/ml) for various durations, cells were then harvested and washed with ice cold PBS and lysed by sonication. 200% g of protein was used for extraction of lipids using Chloroform:Methanol:HCl (100:100:1). The organic phase was dried under nitrogen. Glycosphingolipids were resolved by high performance thin layer chromatography using chloroform/methanol/0.25% KCl (70:30:4, v/v/v) as the developing solvent. The gel area corresponding to LacCer was scraped, and radioactivity was measured employing “liquiscint” (NEN Life Science Products) as a scintillating fluid.

### Identification and Analysis of Purified LacCer.

LacCer from LPS treated cells was resolved by a silica gel-60 TLC plate. Fatty acid methyl ester (FAME) was prepared as described earlier (Khan et al., 1998; Pahan et al., 1998b). FAME was analyzed by gas chromatography (Shimadzu, GC 17A gas chromatograph) on silica capillary column and quantified as a percentage of total fatty acids identified. Mass spectrometry data were recorded as Finnegan LCQ classic (ion trap quadrupole) mass spectrometer.

### GalT-2 Activity Assay.

The activity of GalT-2 was measured using [³H]UDP-galactose as the galactose donor and GlcCer as the acceptor as described previously (Yeh et al., 2001). Briefly, cells were harvested in PBS and cell pellets were suspended in Triton X-100 lysis buffer. Cell lysates were sonicated and following protein quantification, 100 μg of cell lysate was added to reaction mixture containing 20 μM of cacodylate buffer (pH 6.8), 1 mM Mn/Mg, 0.2 mg/ml Triton X-100 (1:2 v/v), 30 mmol of GluCer and 0.1 mmol of UDP-[³H]galactose in a total volume of 100 μl. The reaction was terminated by adding 10 μl of 0.25M EDTA, 10 μl of 0.5M KCl and 500 μl of Chloroform/Methanol (2:1 v/v) and the products were separated by centrifugation. The lower phase was collected and dried under nitrogen. Following resolution on HPTLC plates, the gel was cut out and radioactivity was measured in a scintillation counter. Assay without exogenous GluCer served as blank and their radioactivity counts were subtracted from all respective data points.

### Gal T-2 Antisense Oligonucleotides.

A 20-mer antisense oligonucleotide of the following sequence (5′-CGC TTG AGC GCA GAC ATC TT-3′, SEQ ID NO:1) targeted against rat lactosylceramide synthase (GalT-2) were synthesized by Integrated DNA Technology. A scrambled oligonucleotide (5′-CTG ATA TCG TCG ATA TCG AT-3′, SEQ ID NO:2) was also synthesized and used as control. Cells were counted and plated a day before transfection and the following day were treated with Oligofectamine (Invitrogen)-oligonucleotide complexes (200 nM oligo) under serum free conditions. 48 h following transfection, the protein levels of GalT-2 were analyzed using polyclonal antibodies raised against rat GalT-2 (Abjent Inc., CA). The transfected cells were stimulated with LPS/IFNγ (1 g/ml) and levels of nitric oxide were checked 24 h following stimulation. iNOS mRNA and protein levels were checked at 6 h and 24 h, respectively, following stimulation of transfected cells.

### RT-PCR Amplification.

Following total RNA extraction using TRIzol (GIBCO) as per manufacturer's protocol, single stranded cDNA was synthesized from total RNA. 5 μg total RNA was treated with 2U DNAse I (bovine pancreas, Sigma) for 15 min at room temperature in 18 μl volume containing 1×PCR buffer and 2 mM MgCl₂. It was then inactivated by incubation with 2 μl of 25 mM EDTA at 65° C. for 15 min. 2 μl of random primers were added and annealed to the RNA according to the manufacturer's protocol. cDNA was synthesized in a 50 μl reaction containing 5 μg of total RNA and 50-100U reverse transcriptase by incubating the tubes at 42° C. for 60 min. PCR amplification was conducted in 25 μl of reaction mixture with 1.0 μl of cDNA, 0.5 mM of each primer and under the manufacturer's Taq polymerase conditions (Takara, Takara Shuzo Co. Ltd, Japan). The sequence of primers used for PCR amplification are as follows; iNOS, (Forward-5′ ctccttcaaagaggcaaaaata 3′, SEQ ID NO:3; Reverse-5′ cacttcctccaggatgttgt 3′, SEQ ID NO:4), GalT-2 (Forward-5′ tggtacaagctagaggc 3′, SEQ ID NO:5; Reverse-5′ gcatggcacattgaa C-3′, SEQ ID NO:6), GAPDH (Forward-5′ cgggatcgtggaagggctaatga 3′, SEQ ID NO:7; Reverse-5′ cttcacgaagttgtcattgagggca3′, SEQ ID NO:8), TNFα (Forward-5′ ccgagatgtggaactggcaga g-3′, SEQ ID NO:9; Reverse-5′cggagaggaggctgactttctc-3′, SEQ ID NO:10) and IL-1β (Forward-5′ccacctcaatggacagaacat-3′, SEQ ID NO:11; reverse-5′ccatctttaggaagacacgggt-3′, SEQ ID NO:12). The PCR program included preincubation at 95° C. for 4 min, amplification for 30 cycles at 94° C. for 1 min plus 50° C. annealing for 1 min plus 74° C. extension for 1 min and a final 74° C. for 10 min extension. 10 μl of the PCR products were separated on 1.2% agarose gel and visualized under UV.

### Plasmids, Transient Transfection and FACS Analysis.

Dominant negative ras expression vector (pCMVrasN17) was purchased from BD Biosciences. pEGFP expression plasmid was purchased from Clontech. p110*Δkin, a kinase deficient version of p110 [the catalytic subunit of PI-3K] was obtained from the Tanti et al (Tanti et al., 1996). 3×10⁵ cells/well were cultured in 6-well plates for one day before the transfection. Transfection was performed with plasmid concentration constant (2.5 μg/transfection) and 8 μl of Fugene transfection reagent (Roche Molecular Biochemicals). 24 h after transfection, the cells were placed in serum free media for overnight. Following stimulation for 18 h, the cells were trypsinized, pelleted and the cells pellets were washed cold phosphate buffered saline (PBS) and finally resuspended in 100 μl PBS. The cells were fixed in 70% ethanol at 4° C. for 1 h. Following fixation, cells were pelleted, the cells pellets were washed with PBS three times. The DNA was stained with 7-AAD. Cell cycle analysis was done. Events were acquired using a Becton Dickinson FACS Calibur equipped with a 488 nM argon laser and CellQuest software. pEGFP was acquired using 515-545 nM bandpass filter (FL1) and 7-AAD was acquired using a 670 nM longpass filter (FL3). DNA histograms were generated using Modfit LT software. The collected data were gated for doublet discrimination and pEGFP positive events.

RNA Extraction and cDNA Synthesis.

Following total RNA extraction using TRIzol (GIBCO) as per manufacturer's protocol, single stranded cDNA was synthesized from total RNA. Five μg total RNA was treated with 2U DNAse I (bovine pancreas, Sigma) for 15 min at room temperature in 18 μl volume containing 1×PCR buffer and 2 mM MgCl₂. It was then inactivated by incubation with 2 μl of 25 mM EDTA at 65° C. for 15 min. 2 μl of random primers were added and annealed to the RNA according to the manufacturer's protocol. cDNA was synthesized in a 50 μl reaction containing 5 μg of total RNA and 50-100 U reverse transcriptase by incubating the tubes at 42° C. for 60 min. The sequence of primers used for PCR amplification are as follows; GAPDH (Forward-5′ cgg gat cgt gga agg gct aat ga-3′, Reverse 5′-ctt cac gaa gtt gtc att gag ggc a-3′). The PCR program included preincubation at 95° C. for 4 min, amplification for 30 cycles at 94° C. for 1 min plus 50° C. annealing for 1 min plus 74° C. extension for 1 min and a final 74° C. for 10 min extension. 10 μl of the PCR products were separated on 1.2% agarose gel and visualized under UV.

### Real-Time PCR.

Total RNA isolation from rat spinal cord sections was performed using TRIzol (GIBCO, BRL) according to the manufacturer's protocol. Real-time PCR was conducted using Biorad iCycler (iCycler iQ Multi-Color Real Time PCR Detection System; Biorad, Hercules, Calif., USA). Single stranded cDNA was synthesized from total RNA as described. The primer sets for use were designed (Oligoperfect™ designer, Invitrogen) and synthesized from Integrated DNA technologies (IDT, Coralville, Iowa, USA). The primer sequences for iNOS (Forward-5′gaaagaggaacaactactgct ggt-3′, SEQ ID NO:13; Reverse-5′gaactgagggtacatgctggagc-3′, SEQ ID NO:14), GAPDH (forward-5′cctacccccaatgtatccgttgtg-3′, SEQ ID NO:15; reverse-5′-ggaggaatgggagttgctgttgaa-3′, SEQ ID NO:16), TNFα (forward-5′cttctgtctactgaacttcggggt-3′, SEQ ID NO:17; Reverse-5′tgg aac tga tga gag gga gcc-3′, SEQ ID NO:18), IL-1β (Forward-5′gagagacaagca acgacaaaatcc-3′, SEQ ID NO:19; Reverse-5′ttcccatcttcttctttgggt att-3′, SEQ ID NO:20), and GFAP (Forward 5′-cca agc cag acc tca cag c-3′, SEQ ID NO:21; Reverse 5′-ccg ata cca ctc ttc tgt ttc ttg-3′, SEQ ID NO:22). IQTM SYBR Green Supermix was purchased from BIORAD (BIORAD Laboratories, Hercules, Calif.). Thermal cycling conditions were as follows: activation of DNA polymerase at 95° C. for 10 min, followed by 40 cycles of amplification at 95° C. for 30 sec and 58.3° C. for 30 sec. The normalized expression of target gene with respect to GAPDH was computed for all samples using Microsoft Excel data spreadsheet.

### Induction of SCI in Rats.

Sprague-Dawley female rats (225-250 g weight) were purchased (Harlan laboratories, Durham, N.C.) for induction of SCI. All rats were given water and food pellets ad libitum and maintained in accordance with the ‘Guide for the Care and Use of Laboratory Animals’ of the US Department of Health and Human Services (National Institutes of Health, Bethesda, Md., USA). The inventors have used a clinically relevant weight-drop device for the induction of SCI in rats as described earlier (Gruner, 1992). Briefly, rats were anesthetized by intraperitoneal (i.p.) administration of ketamine (80 mg/kg) plus xylazine (10 mg/kg) followed by laminectomy at T12. While the spine was immobilized with a stereotactic device, injury (30 g/cm force) was induced by dropping a weight of 5 gm from a height of 6 cm onto an impounder gently placed on the spinal cord. Sham operated animals underwent laminectomy only. Upon recovery from anesthesia, animals were evaluated neurologically and monitored for food and water intake. However, no prophylactic antibiotics or analgesics were used in order to avoid their possible interactions with the experimental therapy of SCI.

### Treatment of SCI.

Rats received the glycosphingolipid inhibitor, PDMP (Matreya Inc, Pleasant Gap, Pa.) at various time points post-SCI. PDMP was dissolved in 5% Tween 80 in saline and diluted with sterile saline (0.85% NaCl) at the time of intraperitoneal (i.p.) administration to Sham and SCI rats. Animals (six per group) were randomly selected to form 4 different groups: vehicle (5% Tween 80 in saline) treated sham (laminectomy only) and SCI, and PDMP (20 mg/kg in 5% Tween 80) treated sham and SCI. The first dose of PDMP was administered (10 min, 30 min, 1 h, 2 h and 12 h) post-SCI followed by the second dose at 24 h (Day 1), third dose at 48 h (Day 2) and fourth dose at 72 h (Day 3) post-SCI. Animals were sacrificed under anesthesia 1 h, 4 h, 12 h, 24 h, 48 h and 72 h following treatment.

Assessment of neurological (functional) recovery was performed by an open-field test using the 21-point Basso, Beattie, Bresnahan (BBB) locomotor rating scale (Basso et al., 1996) until Day 15 post-SCI. The animals were observed by a blinded observer before assignment of grade.

### Preparation of Spinal Cord Sections.

Rats were anesthetized and sacrificed by decapitation. Spinal cord sections with the site of injury as the epicenter (lesion epicenter) were carefully extracted from VHC-treated sham and SCI as well as PDMP-treated sham and SCI animals. Tissue targeted to be used for RNA and protein extraction was immediately homogenized in TRIzol (GIBCO, BRL), snap frozen in liquid nitrogen and stored at −80° C. until further use. Total RNA was extracted as per manufacturer's protocol and used for cDNA synthesis as described earlier. Sections of spinal cord to be used for histological examination as well immunohistochemistry were fixed in 10% neutral buffered formalin (Stephens Scientific, Riverdale, N.J.) The tissues were embedded in paraffin and sectioned at 4 μM thickness.

### Immunohistochemical Analysis.

Spinal cord sections were deparaffinized and sequentially rehydrated in graded alcohol. Slides were then boiled in antigen unmasking fluids (Vector Labs, Burlingame, Calif.) for 10 min, cooled in the same solution for another 20 min and then washed 3 times for 5 min each in Tris-sodium buffer (0.1M Tris-HCL, pH-7.4, 0.15M NaCl) with 0.05% Tween 20 (TNT). Sections were treated with Trypsin (0.1% for 10 min) and immersed for 10 min in 3% hydrogen peroxide to eliminate endogenous peroxidase activity. Sections were blocked in Tris sodium buffer with 0.5% blocking reagent (TNB) (supplied with TSA-Direct kit, NEN Life Sciences, Boston Mass.) for 30 min to reduce non-specific staining. For immunofluorescent labeling, sections were incubated overnight with anti-iNOS, TNFα or IL-1β antibody (1:100, mouse monoclonal, Santa Cruz, Calif.) followed by antibodies against the astrocyte marker, GFAP (1:100, rabbit polyclonal, DAKO, Japan) for 1 h (in case of double staining). Anti-iNOS was visualized using fluorescein-isothiocyanate (FITC) conjugated anti-mouse IgG (1:100, Sigma) and GFAP using tetramethylrhodamine isothiocyanate (TRITC) conjugated anti-rabbit IgG (1:100, Sigma). The sections were mounted in mounting media (EMS, Fort Washington, Pa.) and visualized by immunofluorescence microscopy (Olympus) using Adobe Photoshop software. Rabbit polyclonal IgG was used as control primary antibody. Sections were also incubated with conjugated FITC anti-rabbit IgG (1:100, Sigma, St. Louis, Mo.), or TRITC conjugated IgG (1:100) without the primary antibody as negative control. H&E staining was carried out as described by (Kiernan, 1990). Luxol fast blue (LFB) PAS was carried out according to (Lassmann and Wisniewski, 1979).

For immunofluorescent double-labeling, sections were incubated overnight with anti-pERK1/2 antibody (1:100, mouse monoclonal, Cell signaling, CA) followed by antibodies against the astrocyte marker, GFAP (1:100, rabbit polyclonal, DAKO, Japan) for 1 h. Anti-GFAP was visualized using fluorescein-isothiocyanate (FITC) conjugated anti-mouse IgG (1:100, Sigma) and pERK using tetramethylrhodamine isothiocyanate (TRITC) conjugated anti-rabbit IgG (1:100, Sigma). The sections were mounted in mounting media (EMS, Fort Washington, Pa.) and visualized by immunofluorescence microscopy (Olympus) using Adobe Photoshop software. Rabbit polyclonal IgG was used as control primary antibody. Sections were also incubated with conjugated FITC anti-rabbit IgG (1:100, Sigma, St. Louis, Mo.), or TRITC conjugated IgG (1:100) without the primary antibody as negative control.

### Fluorescent TUNEL Assay.

TUNEL assay was carried out using APOPTAG Fluorescein In Situ Apoptosis Detection Kit (Serological Corporation, Norcross, Ga.) according to manufacturer's protocol. For double labeling, sections were incubated with mouse anti-neuronal nuclei 1:100 (NeuN, Chemicon, USA). Sections were incubated with TRITC conjugated mouse IgG 1:100 (Sigma), mounted in mounting media and visualized by fluorescence microscopy.

### Statistical Analysis.

All values shown in the figures are expressed as the means ±SD of obtained from at least three independent experiments. The results were examined by one- and two-way ANOVA; then individual group means were compared with the Bonferroni test. A p value<0.05 was considered significant.

### EXAMPLE 2

### Lactosylceramide is Involved in Gene Expression of iNOS and Other Inflammatory Mediators

In this example, the inventors identified a novel role of LacCer which mediates lipopolysaccharide (LPS) and interferon-γ (IFNγ) induced iNOS gene expression through the Ras/ERK1/2 and Iκ-B/NF-κB pathways. The possible role of GSL and the advantage of inhibition of their synthesis in suppressing inflammation following CNS trauma was demonstrated by observing an inhibition of iNOS, TNFα and IL-1β gene expression and reactive astrogliosis by a GSL biosynthesis inhibitor, D-threo-1-Phenyl-2-decanoylamino-3-morpholino-1-propanol.HCI (PDMP) in a rat model of SCI. Furthermore, PDMP treatment improved the neurological outcome post-SCI and also attenuated SCI-induced neuronal apoptosis. Histological examination of the spinal cord tissue showed marked decrease in SCI-induced white matter vacuolization as well as loss of myelin upon PDMP treatment. This example establishes the role of LacCer as a key signaling modulator in the regulation of iNOS gene expression via regulation of Ras/ERK1/2 and NF-κB pathway. It further demonstrates the effectiveness of PDMP in attenuation of inflammation-mediated secondary damage for amelioration of CNS pathology as in SCI.

LPS/IFNγ-Induced NO Production and iNOS Gene Expression is Mediated by GSL.

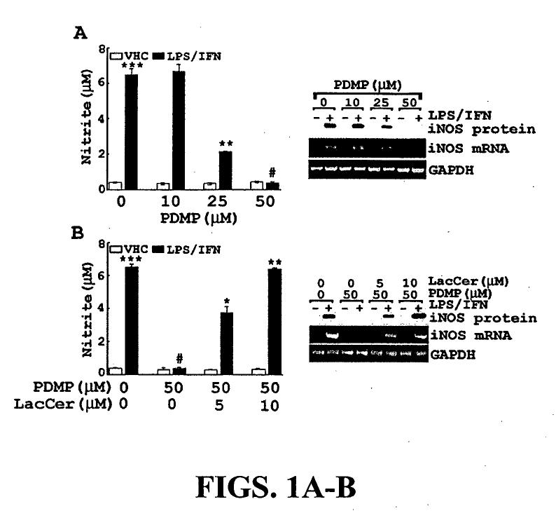

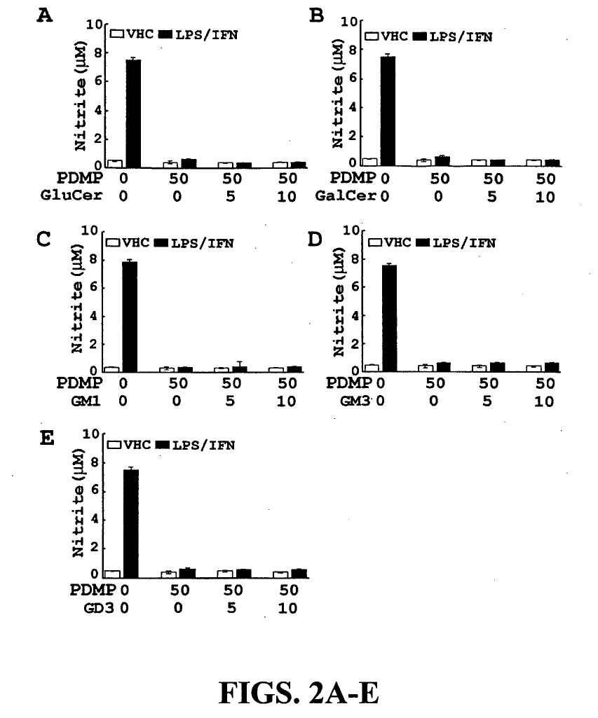

LPS/IFNγ-stimulation of primary astrocytes resulting in iNOS gene expression is a complex multi-step process. In the present example, the possibility that GSL is involved in iNOS induction was tested. Primary astrocytes pretreated for 0.5 h with several concentrations of the glycosphingolipid inhibitor, PDMP (0, 10, 25 and 50 μM), followed by stimulation with LPS/IFNγ (1 μg/ml; 10 U/ml) showed a dose dependent decrease in production of NO as well as mRNA and protein levels of iNOS (FIG. 1A). However, in the presence of increasing doses of LacCer, PDMP-mediated inhibition of NO production and iNOS gene expression (FIG. 1B) was blunted. To prove that this was a LacCer specific effect, other glycosphingolipid derivatives were also exogenously supplemented. However, the presence of GluCer (FIG. 2A), GalCer (FIG. 2B) or gangliosides-GM₁ (FIG. 2C), GM₃ (FIG. 2D) and GD₃ (FIG. 2E) did not reverse PDMP mediated inhibition of LPS/IFNγ-induced NO production as LacCer, provided exogenously, did. These studies indicate that a metabolite of the glycosphingolipid pathway, LacCer, may play a role in the regulation of LPS/IFNγ-mediated induction of iNOS gene expression and NO production.

### LPS/IFNγ-Stimulation Results in Increased Synthesis and Altered Fatty Acid Composition of LacCer.

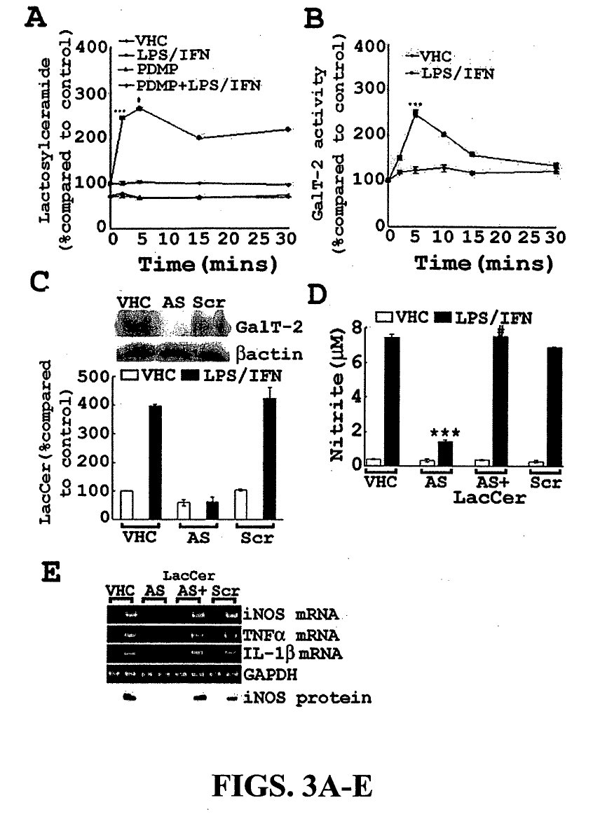

To understand the mechanism of LPS/IFNγ-induced iNOS gene expression by LacCer the in situ levels of lactosylceramide were quantified. [¹⁴C]LacCer was resolved and characterized by Rf value using commercially available standard LacCer by HPTLC as described in Example 1. As shown in FIG. 3A, a sharp increase in [¹⁴C]LacCer levels was observed within 2-5 min following stimulation with LPS/IFNγ. Upon LPS/IFNγ-stimulation, LacCer levels increased ˜1.5 fold of those observed in unstimulated cells. Inhibition of LacCer synthase (GalT-2, enzyme responsible for LacCer biosynthesis) by PDMP inhibited this increase in [¹⁴C]LacCer biosynthesis following LPS/IFNγ-stimulation. Additionally, when GalT-2 activity was assayed following LPS/IFNγ-stimulation, a rapid increase in enzyme activity with peak at 5 min following LPS/IFNγ stimulation was observed (FIG. 3B). The role of GalT-2 and its product LacCer in iNOS gene regulation was further confirmed by silencing GalT-2 gene using antisense (AS) DNA oligomers against rat GalT-2 mRNA and a sequence-scrambled (Scr) oligomer as a control. As shown in FIG. 3C, diminished protein levels of GalT-2 by AS GalT-2 oligonucleotides correlated with diminished synthesis of [¹⁴C]LacCer upon LPS/IFNγ-stimulation. Silencing of GalT-2 with AS oligomers decreased the LPS/IFNγ-mediated NO production (FIG. 3D) and iNOS mRNA and protein levels (FIG. 3E) whereas supplementing LacCer exogenously reversed the inhibition, presumably because the signaling events downstream of LacCer could be triggered upon addition of LacCer. In addition to iNOS, inhibition of LacCer synthesis upon LPS/IFNγ-stimulation by using AS oligonucleotides also suppressed the mRNA expression of two other potent pro-inflammatory cytokines, TNFα and IL-1β (FIG. 3E) both of which are known to be critical players in causing secondary damage by inducing inflammation in neurological disorders (Andersson et al., 1992; Renno et al., 1995; Saklatvala et al., 1996; Perry et al., 2001). The AS-mediated inhibition of the expression of these inflammatory cytokines was also reversed by exogenous supplementation of LacCer suggestive of the fact that along with iNOS expression, LacCer may exacerbate inflammation in general by mediating expression of potent inflammatory mediators.

Furthermore, to investigate the possible role of LacCer and GalT-2 in iNOS gene regulation, the LacCer that was isolated and purified in the above experiments was also investigated for its compositional and structural study. The structure of LacCer obtained from LPS/IFNγ-stimulated cells or from unstimulated cells was studied by its mass spectrometric (MS) analysis. LacCer consisting of 18:0 had the diagnostic peak at m/z 889 (M, 1.1%), m/z 890 (M+H, 1.4%) and m/z 740 (M-[5×OH+2×CH₃OH], 41.6%). Similarly, 16:0 species of LacCer had the significant peaks present at m/z 861 (M+, 0.8%), 862 (M+H, 1.2%), m/z 860 9M—H, 1.1%) and m/z 711 (860-[5×OH+2×CH₃OH], 51.9%). The species of LacCer consisting of oleic acid (18:1) had a significant peak present at m/z 888 (M+H, 1.8%) and m/z 739 (888-[5×OH+2×CH₃OH], 100%). Two more important peaks present were at m/z 342 (M-sphingolipid backbone, 4.4%) and m/z 529 (M-LacCer backbone-H₂O, 1.5%). In addition, LPS/IFNγ-stimulated cells had the altered fatty acid profile measured as % of total fatty acids and compared with the levels of same fatty acid unstimulated cells. GC analysis identified 3 major fatty acids (18:0, 56.2%; 18:1, 26.4%; 16:0, 12.9%) in LPS/IFNγ-stimulated cells. Furthermore, LPS/IFNγ-stimulated cells had increased levels (measured as % of total fatty acids) of saturated fatty acids including 14:0 (167%), 16:0 (65.8%), 18:0 (7.3%) and 20:0 (5.7%) when compared with unstimulated cells. Taken together, the data from the GC and MS confirmed that LacCer from LPS/IFNγ-stimulated cells has 3 major species consisting of stearic, oleic and palmitic acids.

LacCer-Mediated Regulation of LPS/IFNγ-Induced iNOS Gene Expression is ROS Dependent.

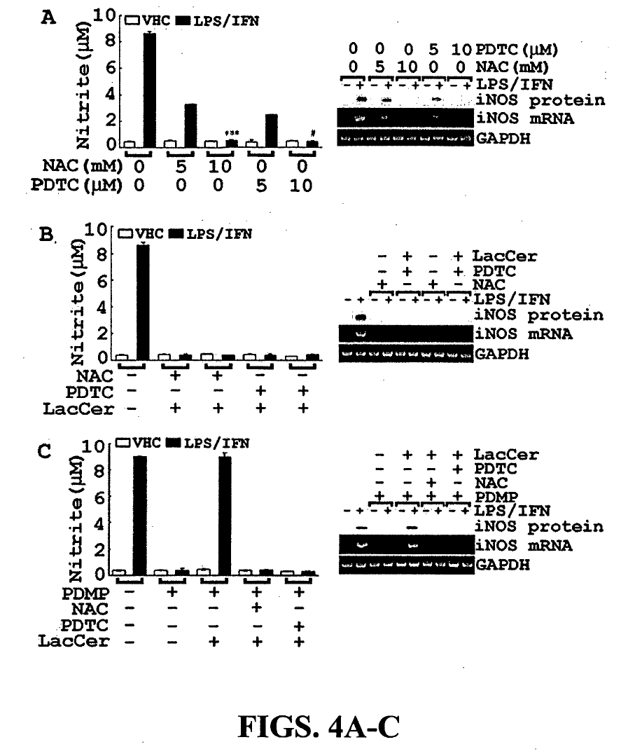

To further elucidate the mechanism of LacCer-mediated regulation of LPS/IFNγ-induced cellular signaling for induction of iNOS expression, the inventors investigated whether these events are reactive oxygen species (ROS) mediated. An earlier report by Pahan et al. (1995) has shown LPS/cytokine-induced iNOS gene expression and NO production to be ROS (e.g H₂O₂, O₂⁻ and OH) mediated, however the source of ROS production has not been clearly defined so far. Furthermore a number of reports have shown that LacCer can stimulate superoxide production and generate oxidative stress in endothelial cells and neutrophils (Bhunia et al., 1997; Iwabuchi and Nagaoka, 2002). In primary astrocytes, pretreatment with increasing concentrations of the membrane-permeant antioxidant N-acetyl cysteine (NAC), a ROS scavenger and precursor for glutathione (Pahan et al., 1998a), and pyrrolidine dithiocarbamate (PDTC), another antioxidant, blocked LPS/IFNγ-induced NO production and iNOS protein and mRNA expression (FIG. 4A). As shown in FIG. 4B, in spite of supplementing LacCer exogenously the NAC- and PDTC-mediated inhibition of LPS/IFNγ-stimulated iNOS gene expression was not reversed. Furthermore, although LacCer could effectively reverse PDMP-mediated inhibition of iNOS gene expression and NO production, however in the presence of NAC and PDTC LacCer was not able to reverse PDMP-mediated inhibition of iNOS expression (FIG. 4C). These results clearly indicate that LacCer regulates LPS/IFNγ-induced iNOS gene expression through a ROS dependent mechanism since in the presence of antioxidants, the signaling cascade is blocked as is iNOS expression.

Activation of Small GTPase Ras and ERK1/2 is Involved in LacCer-Mediated Regulation of LPS/IFNγ-Induced iNOS Gene Expression and is ROS Dependent.

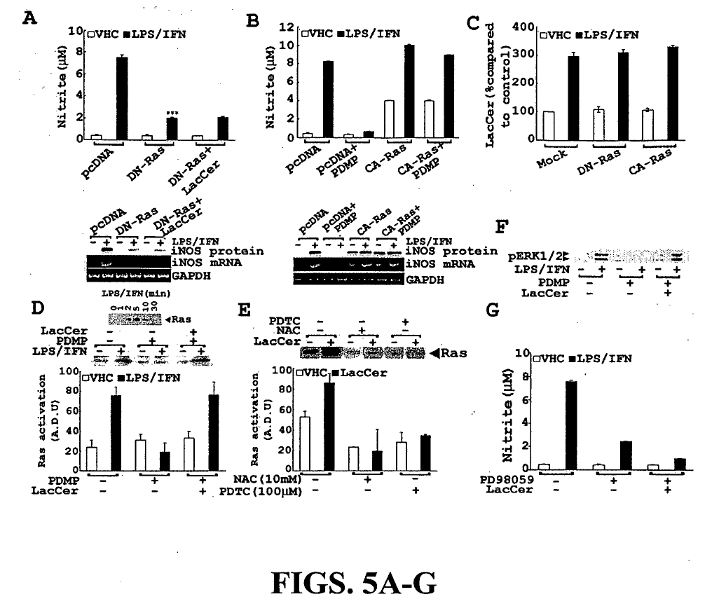

As a recent study indicated that small GTPase Ras is critical for LPS/IFNγ-induced iNOS gene expression (Pahan et al., 2000) compounded with the fact that this protein is redox sensitive (Lander et al., 1995), the role of Ras in LacCer-mediated regulation of iNOS expression was investigated. Transient transfection with dominant negative Ras; DN-Ras (hras N17 mutant) inhibited LPS/IFNγ-mediated iNOS gene expression which could not be reversed by supplementation of exogenous LacCer. The inability of exogenous LacCer to bypass the inhibition by DN-Ras demonstrated that Ras is necessary for LacCer-mediated iNOS gene expression and suggests that Ras is downstream of LacCer in the signaling cascade that induces iNOS expression and NO production (FIG. 5A). Moreover, transient transfection with constitutively active Ras; CA-Ras (bras G12V mutant) completely bypassed PDMP-mediated inhibition of iNOS gene expression and NO production (FIG. 5B) which further substantiated the conclusion that functional Ras downstream of LacCer is critical for mediating induction of iNOS expression. Since neither DN-Ras nor CA-Ras had any effect of LPS/IFN-stimulated [¹⁴C]LacCer synthesis the possible role of Ras in regulating LacCer synthesis was ruled out (FIG. 5C).

As expected the stimulation of cells with LPS/IFNγ enhanced the activation of Ras (maximal activation was detected within 5 min after LPS/IFN treatment; FIG. 5D). This LPS/IFNγ-mediated activation of Ras was reduced by the pretreatment with the GSL inhibitor, PDMP, which was fully reversed by addition of LacCer indicating that LacCer mediates iNOS gene expression by activation of Ras. Furthermore, LacCer-mediated Ras activation was inhibited upon pretreatment with NAC and PDTC thus showing that LacCer-mediated Ras activation is ROS mediated (FIG. 5E). In addition to Ras, activation of ERK1/2 (which are downstream targets of Ras) was also observed upon LPS/IFNγ-stimulation. Pretreatment with PDMP inhibited the LPS/IFNγ-induced phosphorylation of ERK1/2 which was reversed in the presence of exogenous LacCer (FIG. 5F). Additionally, inhibition of a kinase responsible for ERK phosphorylation and activation, MEK1/2, by PD98059 resulted in inhibition of NO production and iNOS expression, further proving the involvement of ERK pathway in iNOS gene expression (FIG. 5G). Supplementation of exogenous LacCer had no effect on PD98059-mediated inhibition of iNOS gene expression thus placing LacCer upstream of the MEK/ERK cascade as a second messenger molecule mediating regulation of LPS/IFNγ-induced iNOS gene expression through this pathway. These findings suggest that LacCer regulates iNOS gene expression through ROS-mediated activation of the small GTPase Ras/MEK/ERK pathway.

The Role of NF-κB in LacCer Mediated Regulation of iNOS Gene Expression.

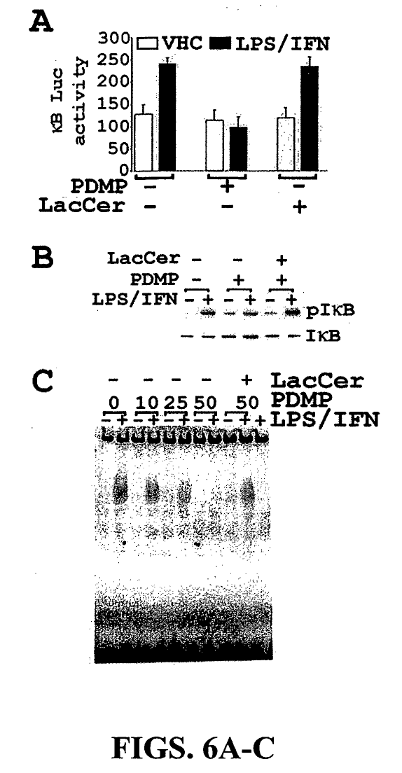

As the activation of NF-κB is necessary for the induction of iNOS ((Xie et al., 1994), and Ras is involved in NF-κB activation resulting in iNOS expression (Pahan et al., 2000), the observed inhibition of LPS/IFNγ-mediated iNOS gene expression by PDMP in rat primary astrocytes may be due to the inhibition of NF-κB. To demonstrate this possibility, the effect of PDMP on luciferase activity was observed in κB-repeat luciferase transfected cells. LPS/IFNγ-induced luciferase activity was abolished upon PDMP pretreatment and was effectively bypassed by exogenously supplemented LacCer (FIG. 6A). As shown in FIG. 6B, NF-κB DNA binding activity tested by electrophoresis mobility shift assay was inhibited by increasing doses of PDMP but was reversed in the presence of exogenous LacCer. Specificity of NF-κB probe binding was proven by using 50× cold probe, which out-competed labeled NF-κB binding activity. As IκB phosphorylation and degradation is required for NF-κB activation and translocation to the nucleus, phosphorylated IκB levels were also examined. Decreased phosphorylation of IκB was observed in the presence PDMP. However, when LacCer was added, the levels of phosphorylated IκB were increased which correlated with increased NF-κB nuclear translocation and DNA binding activity (FIG. 6C). These results show that LacCer may mediate transcriptional regulation of LPS/IFNγ-induced iNOS gene expression through the IκB/NF-κB pathway.

Attenuation of Tissue Destruction and Demyelination by Treatment with PDMP Post-SCI.

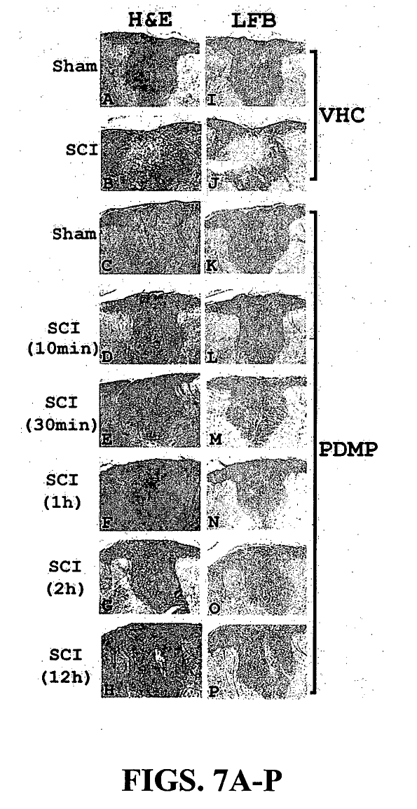

To test the physiological relevance of our observations and further investigate the role of LacCer in the induction of iNOS in neuro-inflammatory diseases, the inventors examined the effect of PDMP in the rat SCI model. PDMP (20 mg/kg) was administered at various time points (10 min, 15 min, 30 min, 1 h, 2 h and 12 h) following SCI and the spinal cord tissue was fixed and analyzed 24 h post-SCI. SCI induced white matter vacuolization and tissue necrosis (FIG. 7B) observed by histological examination of injured rat spinal cord sections was markedly decreased in tissue sections of PDMP-treated SCI rats. Treatment with PDMP 10 min (FIG. 7D), 30 min (FIG. 7E), 1 h (FIG. 7F) and 2 h (FIG. 7F) was efficacious in protecting against tissue damage as compared with VHC-treated SCI (FIG. 7B). Treatment with PDMP 12 h post-SCI showed some damage but was still able to provide a substantial amount of protection against tissue destruction as compared to VHC-treated SCI (FIG. 7H). The weight-drop injury is known to also result in loss of myelin resulting in locomotor dysfunction of the hindlimbs (Suzuki, et al., 2001). LFB staining of spinal cord sections for myelin from VHC-treated SCI rats showed profound demyelination (FIG. 7J) which was also attenuated by PDMP treatment until 12 h post-SCI (FIG. 7P). Taken together these results document that treatment with PDMP protects against white matter vacuolization, tissue destruction and demyelination following SCI and is effective when administered within minutes or until 12 h post-SCI.

### PDMP Treatment Post-SCI Shows Improved Locomotor Function.

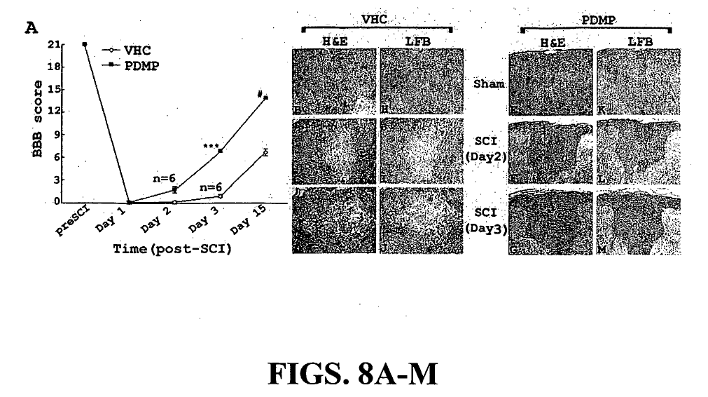

Necrosis and apoptosis which develop in a delayed fashion are reported to play an important role in secondary injury after SCI especially because neurological deficit to a large extent is determined by the lesion size in the white matter (Wrathall, 1992; Wrathall et al., 1996). The locomotor function of rats post SCI was assessed based on the 21 point Basso, Beattie and Bresnahan scale (BBB score) that evaluates various criteria of hind limb mobility post-SCI (Basso et al., 1996). The first dose of PDMP was administered 10 min following SCI, second dose at 24 h (Day 1) post SCI, third dose at 48 h (Day 2) post-SCI and the last dose at 72 h (Day 3) post-SCI. Day 4 until Day 15 post-SCI, the rats were cared for, without treatment and monitored for locomotor functions until Day 15. As shown in FIG. 8A, all animals started with a normal score of 21 pre-spinal cord injury (pre-SCI). The score plummeted to 0 at Day 1 with bilateral hind limb paralysis in all animals following SCI. However, PDMP-treated animals regained hind limb function much sooner than the VHC-treated animals. PDMP-treated rats showed a score of 6.9±0.2 at Day 3 post-SCI which reflects extensive movement of hip, knee and ankle, however, the VHC-treated rats showed profound hind limb paralysis with a score of 0.9±0.2 with no observable hind limb movement. Even when PDMP treatment was stopped at day 3 post-SCI, the PDMP-treated SCI rats steadily gained hind limb function. At day 15 post-SCI, PDMP-treated rats had a BBB score of 13.9±0.1 demonstrating consistent weight supported plantar steps and fore limb-hind limb (FL-HL) coordination. The improved locomotor functions at Day 2 and 3 upon PDMP treatment also correlated with reduced tissue necrosis (FIG. 8F and FIG. 8G; respectively) and demyelination (FIG. 8 L and FIG. 8M; respectively) at the lesion epicenter. Spinal cord sections from VHC-treated rats at Day 2 and 3 post-SCI showed a large necrotic core at the lesion epicenter (FIG. 8C and FIG. 8D; respectively) and profound demyelination (FIG. 8I and FIG. 8J; respectively). The VHC-treated SCI rats showed very slow recovery of hind limb motor function as well with a BBB score of 6.7±0.4 at Day 15 post-SCI. These results clearly demonstrate the efficacy of PDMP in reducing SCI-induced pathology possibly through attenuation of post-SCI inflammation resulting in improved functional outcome.

Efficacy of PDMP in Controlling Inflammation and iNOS Induction in SCI.

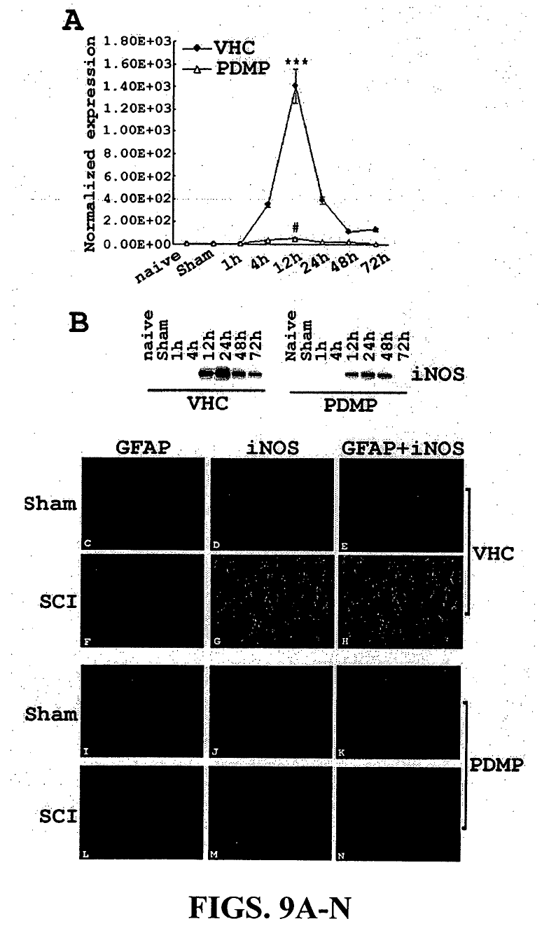

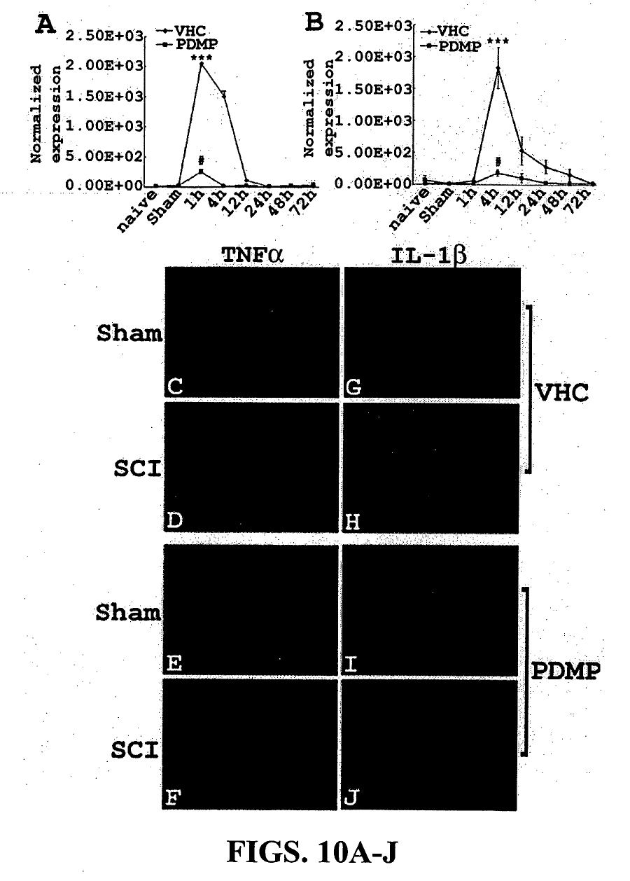

Secondary damage as a result of inflammation in response to primary injury is widely believed to exacerbate the impact of the primary injury and impede neuronal recovery. Inflammation comprising of pro-inflammatory cytokine expression and iNOS, TNFα and IL-1β gene expression resulting in NO production by reactive astrocytes and macrophages significantly contributes to apoptosis, axonal destruction and functional deficit in SCI (Wada et al., 1998a; Wada et al., 1998b). To demonstrate the possibility that protection against white matter destruction and demyelination by PDMP might be through attenuation of iNOS expression, iNOS expression was analyzed post-SCI. As shown in FIG. 9, a robust induction of iNOS mRNA measured by real time PCR (FIG. 9A) and protein expression (FIG. 9B) is observed 12 h following SCI in VHC-treated SCI group as compared to the Naïve or Sham operated animals. PDMP treatment post-SCI markedly suppressed this increase in iNOS gene expression. Double immunofluorescence analysis of spinal cord sections from the lesion epicenter of VHC-treated SCI rats showed a significant increase in GFAP; a marker for reactive astrogliosis (FIG. 9F) and iNOS (FIG. 9G) levels and their co-localization (FIG. 9H) 24 h post-SCI, whereas PDMP-treated SCI rats showed significantly reduced GFAP (FIG. 9L) as well as iNOS (FIG. 9M) expression and their co-localization (FIG. 9N), thus demonstrating the efficacy of PDMP in vivo in attenuating iNOS gene expression as well as reactive astrogliosis. In addition to iNOS induction, treatment with PDMP was equally effective in suppressing the expression of pro-inflammatory cytokines such as TNFα and IL-1β. A robust increase in mRNA levels of TNFα at 1 h (FIG. 10A) and IL-1β 4 h (FIG. 10B) post-SCI was observed which was markedly inhibited upon PDMP treatment. Immunofluorescence detection showed increased protein levels of TNFα (FIG. 10D) and IL-1β (FIG. 10H) post-SCI in the VHC-treated SCI group which was significantly suppressed upon PDMP treatment (FIG. 10F and FIG. 10J, respectively). These studies demonstrate the efficacy of PDMP in attenuating iNOS expression by reactive astrocytes at the site of lesion in an in vivo model of SCI. In addition to iNOS, PDMP also attenuated the production of pro-inflammatory cytokines such as TNFα and IL-1β, both of which initiate deadly cascades causing neuronal apoptosis and massive secondary injury in SCI. The observed anti-inflammatory potential of PDMP finds critical relevance in a number of other neuroinflammatory diseases as well since iNOS, TNFα and IL-1β expression and their related pathology is common to a number of CNS diseases.

Attenuation of Apoptosis and Demyelination by Attenuation of iNOS Gene Expression Post-SCI by PDMP.

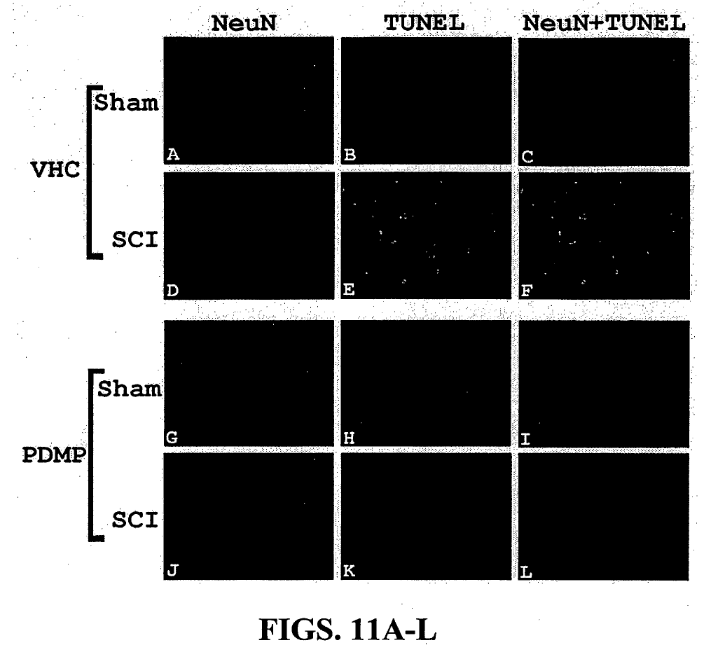

With respect to spinal cord impairment following trauma at the molecular level, NO has been reported to be closely involved in the development of post-traumatic cavitation, neuronal death, axonal degeneration and myelin disruption. Significantly numerous TUNEL-positive cells were scattered in the lesion epicenter post-SCI (FIG. 11E) which were identified to be neurons by double immunofluorescence staining using anti-neuronal nuclei (NeuN) antibodies (FIG. 11D and FIG. 11F). PDMP had a dual beneficial effect in the rat model of SCI. It could attenuate iNOS and pro-inflammatory cytokines expression post-SCI and furthermore as shown in FIG. 11J, FIG. 11K and FIG. 11L also provided protection against apoptosis of neurons. This is of significant importance as no adverse effect of PDMP was observed on neuronal survival in sham operated animals (FIG. 11G, FIG. 11H and FIG. 11I) showing that the dose administered effectively attenuated inflammation without any obvious adverse effects which also translates in reduced SCI-related pathology in terms of neuronal loss. Taken together these studies document the anti-inflammatory potential of PDMP in SCI and possibly other neuroinflammatory disorders since it can effectively block inflammatory events such as iNOS and cytokine expression thus providing protection against white matter vacuolization, neuronal apoptosis and demyelination.

### Discussion

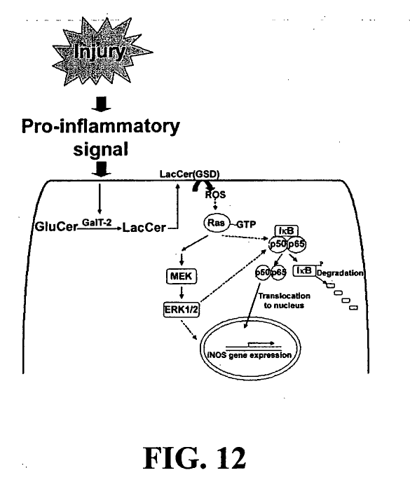

Nitric-oxide mediated pathophysiology is common to a number of neuroinflammatory diseases including stroke and spinal cord injury (SCI). Since the factors that induce and regulate iNOS gene expression in inflammatory diseases are not completely known, in the above experiments the inventors investigated the involvement of GSL and demonstrated a novel pathway of iNOS gene regulation by LacCer-mediated events involving Ras/ERK1/2 and the IκB/NF-κB pathways in primary astrocytes. These conclusions are based on the following findings. (1) LPS/IFNγ-stimulation induced the activity of GalT-2 and increased the production of LacCer. (2) The inhibition of GSL synthesis by PDMP or antisense oligoucleotides to GalT-2 inhibited iNOS gene expression which was reversed by LacCer but not other GSLs (GluCer, GalCer, GM₁, GM₃ and GD₃). (3) Inhibition of LacCer synthesis also inhibited the activation of Ras, ERK and NF-κB pathway. (4) LacCer-stimulated activation of the Ras/ERK signaling cascade was found to be necessary and ROS dependent as the presence of antioxidants, NAC and PDTC, abolished LacCer-mediated Ras activation as well as LPS/IFNγ-stimulated iNOS expression. FIG. 12 shows a schematic representation of the possible regulation of the Ras/ERK/NF-κB pathways by LacCer. Activation of the small GTPase Ras could be through the direct activation of Src kinases associated with the LacCer-enriched glycosphingolipid signaling domains (GSD) present on the cell surface. A number of studies have shown that several transducer molecules such as Src kinase, associate with these GSD and form functional units which mediate signal transduction and cellular functions (Brown and London, 1998). In particular, a Src kinase, Lyn, has been found to directly associate with LacCer resulting in superoxide generation via NADPH oxidase activation in neutrophils (Iwabuchi and Nagaoka, 2002). Src kinase activation possibly leading to ROS generation may be followed by Grb/SOS-mediated Ras activation that triggers the downstream, MEK1/2-ERK1/2 pathway. Activation of the small G-protein Ras and the downstream ERK1/2 has been demonstrated earlier to mediate cytokine induced iNOS gene expression and NF-κB activation (Pahan et al., 1998b; Marcus et al., 2003). Since Ras-mediated NF-κB regulation has been demonstrated earlier (Won et al., 2004), LacCer-mediated activation of the IκB-NFκB pathway could well be mediated by Ras activation. The critical role for NF-κB in the transcriptional regulation of iNOS gene expression via phosphorylation and degradation of Iκ-B has been demonstrated earlier (Pahan et al., 1998b). Furthermore, the potentiation of cytokine-mediated expression of iNOS by sphingolipids has been well documented (Pahan et al., 1998b; Giri et al., 2002). The data presented in this example identify a glycosphingolipid, LacCer, as a signaling molecule regulating iNOS gene expression. In addition, the blockade of SCI-mediated iNOS and pro-inflammatory cytokines' gene expression in the rat SCI model by PDMP further establishes LacCer, generated through GalT-2 stimulation, to be a potent signaling lipid molecule that triggers inflammation and mediates NO-mediated pathophysiology in various neuroinflammatory diseases.

Since the discovery of the sphingomyelin (SM) cycle, which involves sphingomyelin hydrolysis by sphingomyelinases (SMases) resulting in ceramide generation, several inducers (1a,25-dihydroxyvitamin D3, radiation, antibody crosslinking, TNFα, IFNγ, IL-1β, nerve growth factor and brefeldin A) have been shown to be coupled to sphingomyelin-ceramide signaling events (Hannun, 1994; Kolesnick et al., 1994; Kanety et al., 1995; Linardic et al., 1996). Ceramide thus generated plays a role in growth suppression and apoptosis in various cell types including glial and neuronal cells (Brugg et al., 1996; Wiesner and Dawson, 1996). Impairment of mitochondrial function results in enhanced production of reactive oxygen species (ROS) and decrease in mitochondrial glutathione levels. Depletion of glutathione has been established as one of the major causes of ceramide-induced cytotoxicity/apoptosis in CNS (Singh et al., 1998). Ceramide generated as result of neutral sphingomyelinase activation has been shown to potentiate LPS- and cytokine-mediated induction of iNOS in astrocytes and C6 glioma cells (Pahan et al., 1998b). Furthermore, ceramide generation and its mediated iNOS gene expression is known to be through the Ras/ERK/NF-κB pathway which is shown to be a redox sensitive process (Pahan et al., 1998b; Singh et al., 1998). Instead of viewing enzymes of sphingolipid metabolism as isolated signaling modules, these pathways are now accepted to be highly interconnected with the product of one enzyme serving as a substrate for the other. This is also true of ceramide generated through the SM cycle or de novo as ceramide can be converted into other bioactive molecules such as sphingosine, sphingosine-1-phosphate or glycosphingolipids. The complexity of these bioactive sphingolipids is accentuated by growing evidence of the presence of ceramide and other derivatives such as LacCer and gangliosides in lipid-enriched microdomains within membranes. These microdomains, called ‘lipid rafts’, have a number of receptors and signaling molecules clustered within or associated with them thus making them hotspots for signaling events (Hakomori and Handa, 2003). The metabolic interconnections of ceramide and other lipids mediators such as sphingosine, sphingosine-1-phosphate (S-1-P) and glycosphingolipids make predicting the specific actions of these intermediates and the enzymes regulating their levels rather complex. For example, while sphingosine has pro-apoptotic effects like ceramide depending on cell type (Spiegel and Merrill, 1996) its rapid conversion to S-1-P has proliferative properties antagonistic to those of sphingosine and ceramide (Spiegel and Milstien, 2000). Of the GSL, GluCer and LacCer, have been shown to promote the drug resistance state (Liu et al., 1999) and to mediate oxidized-LDL and TNFα effects on superoxide formation, the activation of MAP kinase and the induction of proliferation in aortic smooth muscle cells respectively (Bhunia et al., 1996; Chatterjee et al., 1997; Bhunia et al., 1998; Chatterjee, 1998).

Traumatic SCI results in pathophysiological changes, that can be characterized as acute, secondary and chronic, that extend from minutes to years after the injury. Numerous detrimental events occur in the acute phase that begins at the moment of injury and extends over the first few days. Mechanical lesions induce immediate damage to the neuronal tracts; blood flow is reduced creating substantial ischemia along with production of potent pro-inflammatory cytokines such as TNFα and IL-1β. In the secondary phase of tissue damage which occurs over a time course of minutes to weeks after injury, increased production of ROS and RNS (reactive nitrogen species), excessive release of excitatory neurotransmitters and inflammatory reactions occur. In addition to massive ischemic necrosis, apoptotic cell death is also observed. The size and GFAP content of astrocytes increases in a process of reactive astrogliosis (Bareyre and Schwab, 2003). Traumatic injury also leads to a strong inflammatory response with the recruitment of peripheral derived immune cells. As with most neurodegenerative conditions including SCI, therapies using NOS inhibitors and antioxidants aimed at preserving the spared tissue after injury along with blocking the ensuing inflammation and apoptosis have shown profound beneficial effects on the behavioral outcome and recovery following injury since these are able to suppress the acute inflammatory reactions and minimize secondary damage (Blight, 1983; Young, 1993; Liu et al., 1997). However, therapies so far have aimed at inhibiting individual events. In this report the inventors found that PDMP treatment post-SCI resulted in profoundly improved hind limb functional outcome (FIG. 8A). PDMP treatment was found effective in 1) blocking trauma-mediated iNOS gene expression in the spinal cord in the rat model of SCI 2) attenuation of pro-inflammatory cytokine production 3) attenuation of reactive astrogliosis evident by reduced GFAP immunoreactivity and 4) marked decrease in neuronal apoptosis and demyelination. Protection of neuronal apoptosis could well be due to inhibition of iNOS expression and NO production. In addition to that, protection against apoptosis may possibly be through depletion of GD₃, a LacCer derived ganglioside, as well. GD₃ is a minor ganglioside in normal adult brains however, its levels are elevated in activated microglia and reactive astrocytes (Kawai et al., 1994). Increased GD₃ has been found in multiple sclerosis plaques (Yu et al., 1974) and in brain tissue from patients with various neurodegenerative disorders, such as Creutzfeld-Jacob disease, and subacute sclerosis encephalitis (Ando et al., 1984; Ohtani et al., 1996). It is now known that GD₃ causes apoptosis of murine cortex neurons (Simon et al., 2002) and murine primary oligodendrocytes (Castro-Palomino et al., 2001). However, in contrast to the toxicity related to GD₃, GM₁-another LacCer derived ganglioside, is known to be essential for neuronal survival (Inokuchi et al., 1998). Since the inventors did not observe a reversal of PDMP-mediated iNOS gene expression by GM₁, the inventors expect PDMP therapy along with GM₁ administration will be effective in bypassing the inflammatory reaction while preserving the GM₁-mediated pro-survival signals.

In conclusion, the above experiments documents a tight link of LacCer with regulation of iNOS gene expression in inflammatory disease processes and unravels a novel, potential therapeutic target of glycosphingolipid modulation for amelioration of pathophysiology in neuroinflammatory disorders.

### EXAMPLE 3

### Lactosylceramide is Involved in Astrogliosis Following Neurotrauma

In this example the inventors investigated the role of two bioactive metabolites of ceramide, sphingosine-1-phosphate and glycosphingolipids (GSLs) in TNFα-induced astrocyte proliferation and reactivity. Results presented in this example demonstrate the involvement of both S1P and LacCer in TNFα-induced astrocyte proliferation. TNFα-stimulation induced LacCer synthase (GalT-2) activation and LacCer synthesis. LacCer-mediated proliferation was through activation of Ras/MEK/ERK pathway. TNFα-induced GalT-2 activation was regulated through SIP-mediated PI-3K activation. Furthermore, PDMP treatment was efficacious in attenuating pathological ERK1/2 activation and astrogliosis in a rat model of spinal cord injury (SCI). This example demonstrates the role of LacCer-mediated regulation of TNFα-induced proliferation of primary astrocytes and a phosphatidylinositol-3K (PI-3K)-mediated regulation of GalT-2 enzyme activity. These results exemplify the importance and efficacy of modulating the GSL pathway in suppressing astrogliosis in SCI which finds relevance in numerous other CNS disorders.

### TNFα-Induced Proliferation of Rat Primary Astrocytes is Mediated by GSL.

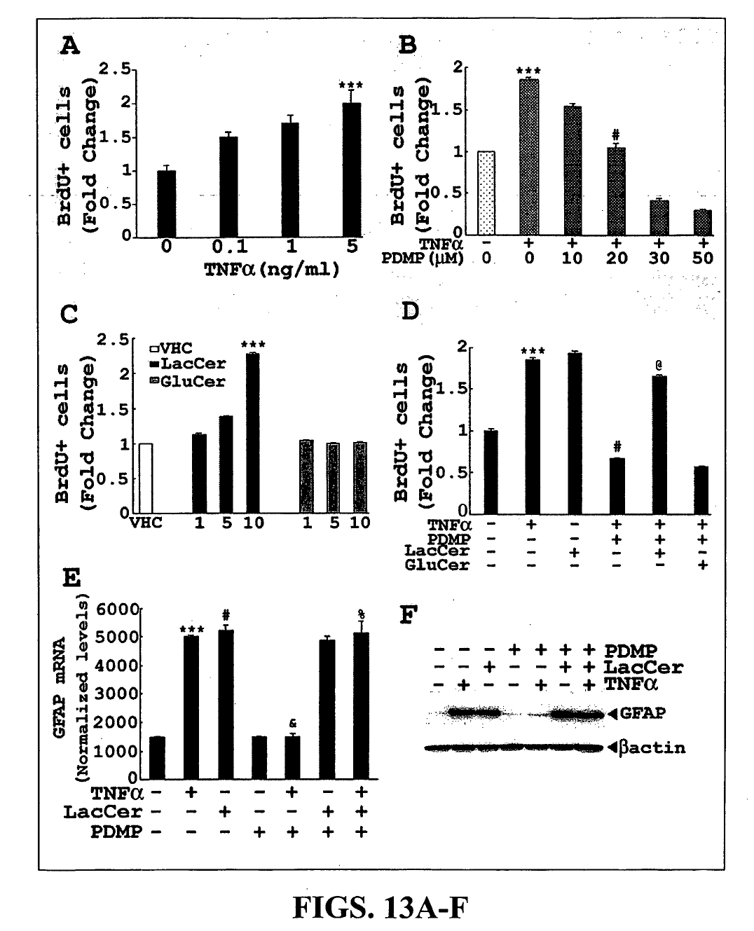

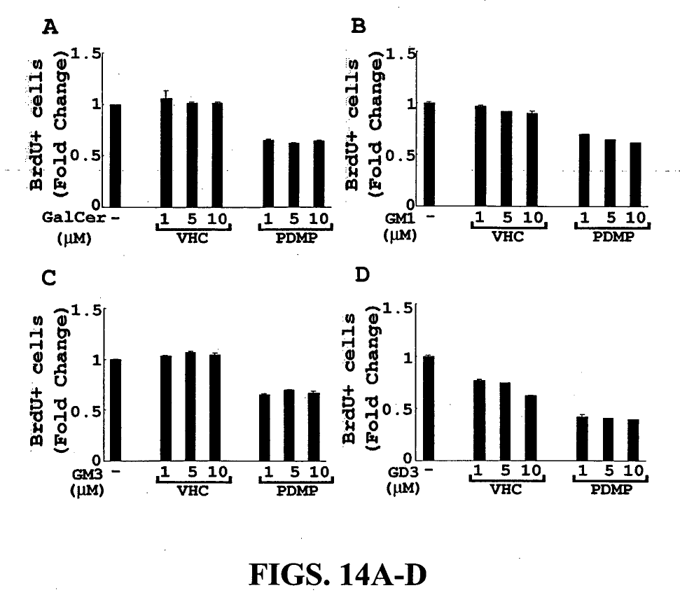

TNFα-stimulation of primary astrocytes resulting in proliferation of astrocytes and their reactive transformation characterized by increased glial fibrillary acidic protein (GFAP) expression is a complex multi-step process. In the present example, the inventors tested whether GSL were somehow involved in proliferation. Increasing concentrations of TNFα (0, 0.1, 1 and 5 ng/ml) induced proliferation of astrocytes which was assayed by BrdU incorporation (FIG. 13A). To address the involvement of GSL in TNFα-mediated proliferation, primary astrocytes were pretreated for 0.5 h with several concentrations of the glycosphingolipid inhibitor PDMP (0, 10, 20 and 30 and 50 μM) followed by stimulation with TNFα (1 ng/ml) for 18 h. PDMP dose dependently inhibited cellular proliferation assayed by BrdU incorporation (FIG. 13B). TNFα at a concentration of 1 ng/ml and PDMP (25 μM) were used for subsequent studies. Furthermore, increasing doses of lactosylceramide (LacCer) induced proliferation of astrocytes, however, glucosylceramide (GluCer) did not have a similar effect (FIG. 13C). Additionally, exogenously supplemented LacCer but not GluCer was able to bypass PDMP-mediated inhibition of TNFα-induced proliferation (FIG. 13D). A similar trend was observed with regard to GFAP gene expression. Pretreatment of astrocytes with PDMP inhibited TNFα-induced GFAP mRNA and protein expression which was reversed by exogenously supplemented LacCer (FIGS. 13E and F). Furthermore, as shown in FIG. 2, exogenous supplementation of other GSL metabolites such as GalCer (FIG. 14A), gangliosides GM1 (FIG. 14B), GM3 (FIG. 14C) and GD3 (FIG. 14D) neither induced proliferation themselves nor could they reverse the PDMP-mediated inhibition of TNFα-induced proliferation thus proving this to be a LacCer specific effect. Therefore, a metabolite of the glycosphingolipid pathway, LacCer, may play a role in the regulation of TNFα-mediated proliferation of astrocytes and GFAP expression, two processes which encompass astrogliosis.

### TNFα-Stimulation Results in Altered Levels of LacCer.

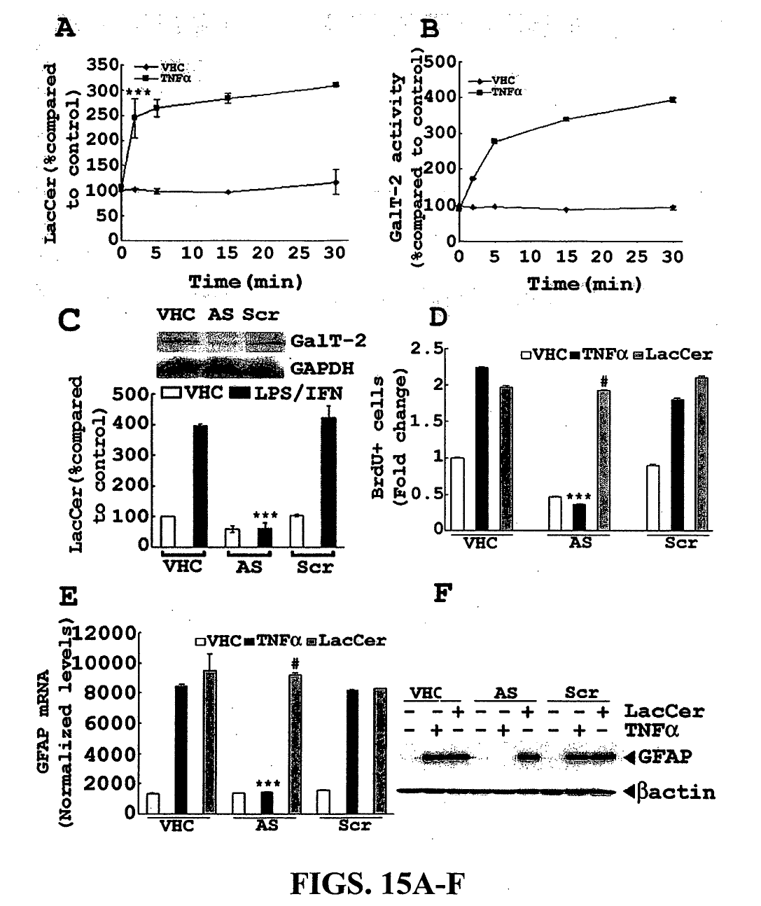

To understand the mechanism of TNFα-induced astrocyte proliferation mediated by LacCer the in situ levels of lactosylceramide were quantified. [¹⁴C]LacCer was resolved and characterized by Rf value using commercially available standard LacCer by HPTLC as described in Example 1. As shown in FIG. 3A, a sharp increase in LacCer levels was observed within 2-5 min following stimulation with TNFα. Upon TNFα-stimulation, LacCer levels increased ˜2.5 fold of those observed in unstimulated cells. Correspondingly, a rapid increase in GalT-2 enzyme activity was also observed upon TNFα stimulation (FIG. 15B). The role of GalT-2 and its product LacCer in cell proliferation was further confirmed by silencing GalT-2 gene using antisense (AS) DNA oligomers against rat GalT-2 mRNA and a sequence-scrambled (Scr) oligomer as a control. As shown in FIG. 15C, diminished protein levels of GalT-2 by AS GalT-2 oligonucleotides correlated with diminished synthesis of [¹⁴C]LacCer upon TNFα-stimulation. Silencing of GalT-2 with AS oligomers decreased the TNFα-induced astrocyte proliferation (FIG. 15D) whereas supplementing LacCer exogenously bypassed the inhibition, presumably because the signaling events downstream of LacCer can be triggered upon addition of LacCer. Correlating with decreased astrocyte proliferation, diminished GFAP mRNA (FIG. 15E) and protein levels (FIG. 15F) were observed upon GalT-2 silencing using GalT-2 antisense oligomers. However, in the presence of exogenous LacCer AS-mediated inhibition of GFAP expression was blunted, thus further establishing the involvement of LacCer in astrogliosis.

### Activation of Small GTPase Ras and ERK1/2 is Involved in LacCer Mediated Regulation of TNFα-Induced Proliferation.

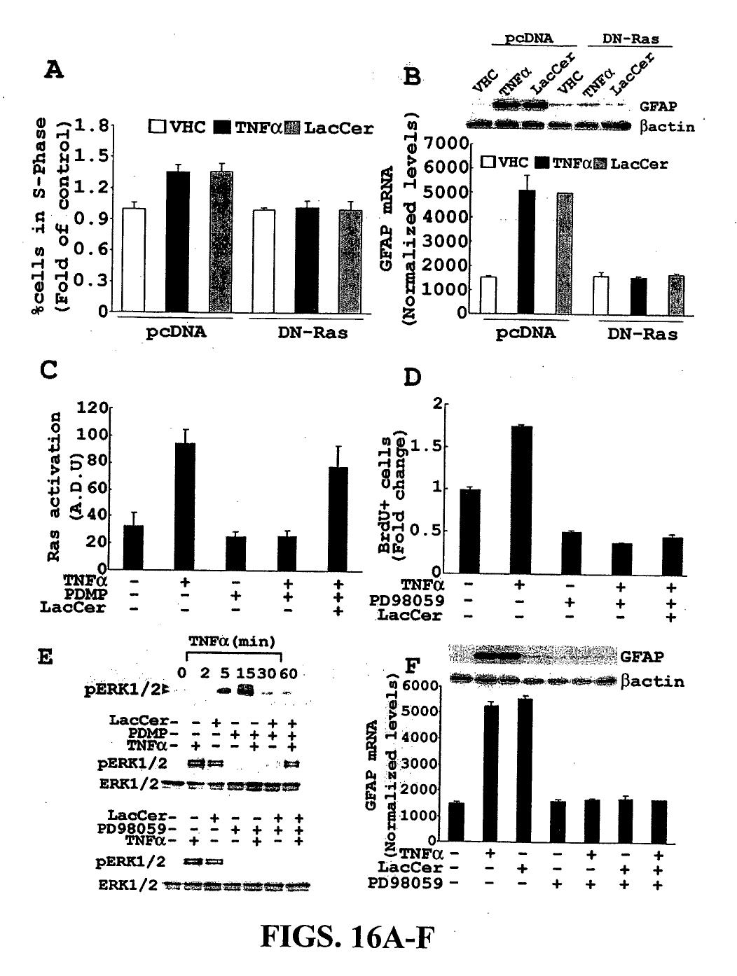

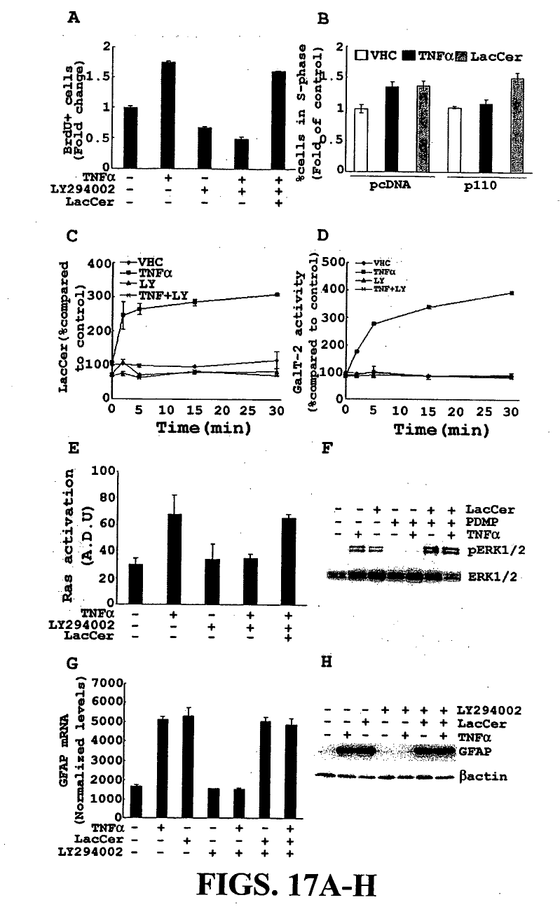

Because a redox-dependent regulation of small GTPase Ras by LacCer was previously observed by the inventors, the possible involvement of Ras in LacCer-mediated regulation of TNFα-induced astrocyte proliferation was investigated. Primary astrocytes were transiently co-transfected with dominant negative Ras; DN-Ras (hras N17 mutant) and pEGFP as a transfection marker followed by cell cycle analysis of the GFP gated cells by FACS. Upon TNFα and LacCer stimulation the percentage of cells in S-Phase was significantly increased in the mock transfected group, however, the DN-Ras transfected group significantly decreased the percentage of cells in S-phase (FIG. 16A). TNFα- and LacCer-induced GFAP mRNA and protein expression was also significantly attenuated in DN-Ras transfected cells and GFAP expression (FIG. 16B). These results show that Ras is involved and necessary for cellular proliferation as well as for GFAP expression. The inability of exogenous LacCer to bypass the inhibition by DN-Ras demonstrated that Ras is necessary for LacCer-mediated proliferation and GFAP gene expression and suggests that Ras is downstream of LacCer in the signaling cascade that induces astrogliosis. The role of Ras was further confirmed by assaying Ras activity using the GST-conjugated Raf-1 RBD (Ras binding domain). As expected, TNFα-stimulation enhanced the activation of Ras which was attenuated upon PDMP pretreatment. PDMP-mediated inhibition of TNFα-induced Ras activation was fully reversed by addition of LacCer further confirming LacCer-mediated regulation of Ras activation. To further examine the signaling events downstream of Ras which mediate proliferation and GFAP expression, the inventors investigated the involvement of two well established downstream effectors of Ras, the extracellular signal-regulated kinases 1 & 2 (ERK1/2) (FIGS. 16A-F) and the phosphatidylinositol 3-kinase (PI-3K) (FIGS. 17A-H). Pretreatment with PD98059 (25 μM), a MEK 1/2 inhibitor, inhibited TNFα-mediated astrocyte proliferation and this inhibition could not be reversed even by exogenous supplementation of LacCer, indicative of MEK-ERK1/2 being downstream of LacCer in the signaling cascade (FIG. 16D). The effect of MEK 1/2 inhibitor observed on cell proliferation was also confirmed by examining ERK1/2 activation using antibodies specific for the phosphorylated (activated) form of ERK1/2. TNFα-induced phosphorylation of ERK1/2 was inhibited both by PDMP and MEK1/2 inhibitor PD98059. However, exogenous LacCer supplementation could only reverse PDMP-mediated inhibition of ERK1/2 activity and not PD98059-mediated (FIG. 16E). This confirmed MEK1/2 and the ERK1/2 kinases to be downstream of LacCer in the signaling cascade that induces astrocyte proliferation. Since earlier reports have documented the involvement of ERK1/2 in regulation of GFAP expression (Zhang et al., 2000) the effect of PD98059 on GFAP expression was also analyzed. In correlation with the effect on proliferation and regulation of GFAP expression (reported earlier), inhibition of the ERK1/2 pathway by PD98059 inhibited GFAP mRNA and protein expression (FIG. 16F). These results establish LacCer-mediated regulation of astrocyte proliferation and GFAP expression to be through the small GTPase Ras/ERK1/2 pathway.

### The Role of PI-3K in TNFα-Mediated Regulation of Astrocyte Proliferation.

The involvement of the second effector of Ras, PI-3K, in astrocyte proliferation was also examined. PI-3K has been reported to be involved in cell survival pathways and proliferation in various cells types including primary astrocytes (Pebay et al., 2001). Pretreatment with LY (30 μM), a PI-3K inhibitor, significantly attenuated TNFα-induced proliferation of primary astrocytes (FIG. 17A). Transient transfection with p110*Δkin, a kinase deficient version of p110 [the catalytic subunit of PI-3K] (Tanti et al., 1996), significantly reduced the percentage of cells in S-Phase upon TNFα-stimulation (FIG. 17B). However, in the presence of LacCer, the LY and p110*Δkin mediated-inhibition of astrocyte proliferation was effectively blunted. The reversal of LY and p110*Δkin induced inhibition by exogenous LacCer shows a differential location of the ERK1/2 kinases and PI-3K in the signaling cascade triggered by LacCer resulting in astrocyte proliferation. Reversal of the effect of PI-3K inhibition by LacCer suggested PI-3K to be upstream of LacCer whereas the non-reversal of MEK1/2-inhibition by LacCer indicated ERK1/2 to be downstream of LacCer. To further understand the role of PI-3K in the mechanism of TNFα-mediated regulation of proliferation, the inventors examined the possibility that PI-3K might be involved in the regulation of LacCer generation in response to TNFα stimulation. Pretreatment with LY inhibited TNFα-induced LacCer synthesis (FIG. 17C) which correlated with inhibition of TNFα-induced GalT-2 activation as well (FIG. 17D). These results suggest two things, first, PI-3K is involved in TNFα-mediated astrocyte proliferation and second, PI-3K is involved in regulation of GalT-2 activity and LacCer synthesis. Since not much is presently known about the post-translational modifications of GalT-2 that might regulate its activity, the involvement of PI-3K offers some clues about the mechanism. Furthermore, pretreatment with LY inhibited TNFα-mediated Ras (FIG. 17E) and ERK1/2 activation (FIG. 17F) that was bypassed by exogenously supplied LacCer. LY also inhibited TNFα-mediated GFAP mRNA (FIG. 17G) and protein expression (FIG. 17H) which was effectively bypassed by exogenously supplied LacCer. These results taken together demonstrated the involvement of PI-3K in regulation of GalT-2 activation and LacCer biosynthesis in response to TNFα stimulation. Through the regulation of LacCer synthesis it regulates the downstream signaling events such as activation of the Ras/ERK1/2 signaling cascade which regulates proliferation and GFAP expression.

### TNFα-Induced PI-3K Activation is Mediated by SIP.

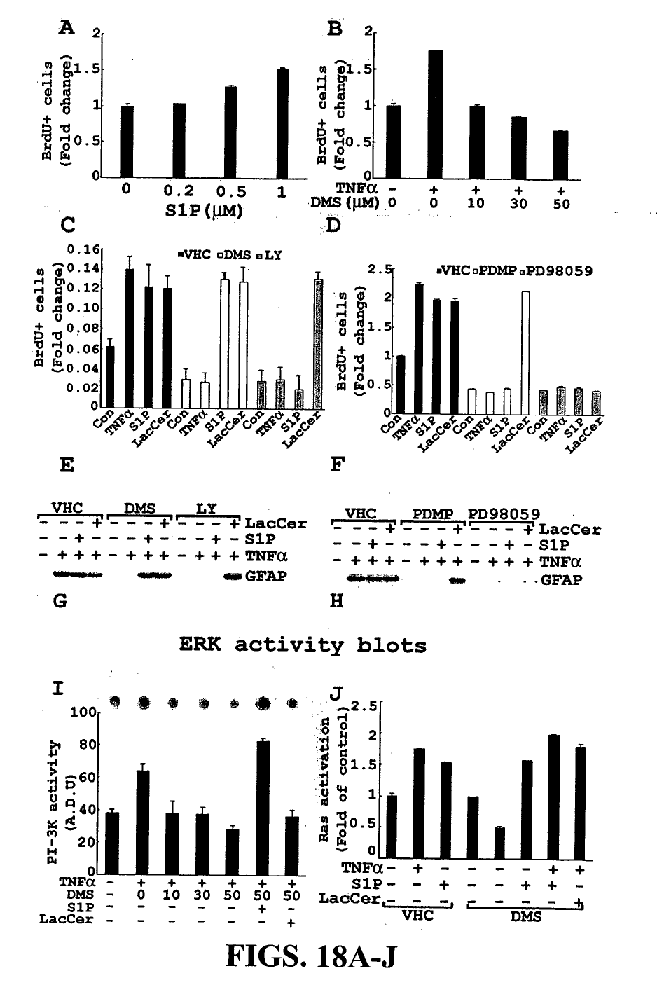

To further elucidate the mechanism of PI-3K activation in response to TNFα the possibility that SIP was somehow involved was investigated since SIP is known to be a potent activator of PI-3K in various cell (Banno et al., 2001; Osawa et al., 2001; Davaille et al., 2002). Increasing concentrations of S1P induced astrocyte proliferation (FIG. 18A). However, pretreatment with increasing doses of dimethysphingosine (DMS), a sphingosine kinase inhibitor, inhibited TNFα-mediated proliferation (FIG. 18B). Furthermore, as shown in FIG. 18C, DMS-mediated inhibition of TNFα-induced astrocyte proliferation was reversed upon exogenously supplementing SIP and LacCer indicating the possibility that LacCer is downstream of S1P. Additionally, LY-mediated inhibition of TNFα-induced proliferation could only be reversed by exogenously supplied LacCer but not SIP thus showing that PI-3K is downstream of S1P. Furthermore, PDMP-mediated inhibition of TNFα-induced proliferation could not be reversed by supplementation of S1P (FIG. 18D). This inhibition could, however, be reversed by exogenously supplementation of LacCer, thus showing that the proliferation observed in response to SIP is in fact mediated through LacCer since exogenous supplementation of LacCer could reverse PDMP-induced inhibition of TNFα-S1P-mediated proliferation. Finally, inhibition TNFα-induced proliferation was completely abrogated by PD98059 and could not be reversed by either SIP or LacCer thus proving that ERK1/2 is the effector downstream of all these bioactive mediators that mediates astrocyte proliferation (FIG. 18D). The trend observed for astrocyte proliferation correlated with GFAP expression whereby DMS-mediated inhibition of TNFα-induced GFAP expression was reversed by S1P and LacCer, however, LY-mediated inhibition was reversed only by LacCer (FIG. 18E). Furthermore, in the presence of LacCer PDMP-mediated inhibition of GFAP expression was blunted, however, it had no effect on PD98059-mediated inhibition (FIG. 18F). To more clearly establish the involvement of S1P in PI-3K activation, PI-3K activity was assayed as described in Example 1. Pretreatment with increasing concentrations of DMS inhibited TNFα-induced PI-3K activation. However, only exogenous supplementation of S1P was able to reverse DMS-mediated inhibition of PI-3K activation. Exogenously supplemented LacCer could not reverse DMS-mediated inhibition of PI-3K activity thus showing that LacCer is not involved in PI-3K activation and that this is a S1P specific effect. The correlation between S1P mediated PI-3K activation and the downstream signaling cascade was further established by examining the effect of DMS on Ras activation. As shown in FIG. 18G, TNFα-induced Ras activation was inhibited upon DMS pretreatment. However, supplementation of S1P reverses DMS-mediated inhibition since the signaling events downstream of S1P can be restored. Additionally, as expected DMS-mediated inhibition of Ras activation was reversed by exogenously supplemented LacCer as well. These studies clearly establish SIP-mediated activation of PI-3K which further regulates LacCer synthesis and initiation of the signaling cascade involved in triggering astrogliosis.

### Efficacy of PDMP in Attenuation of Astrogliosis in SCI.

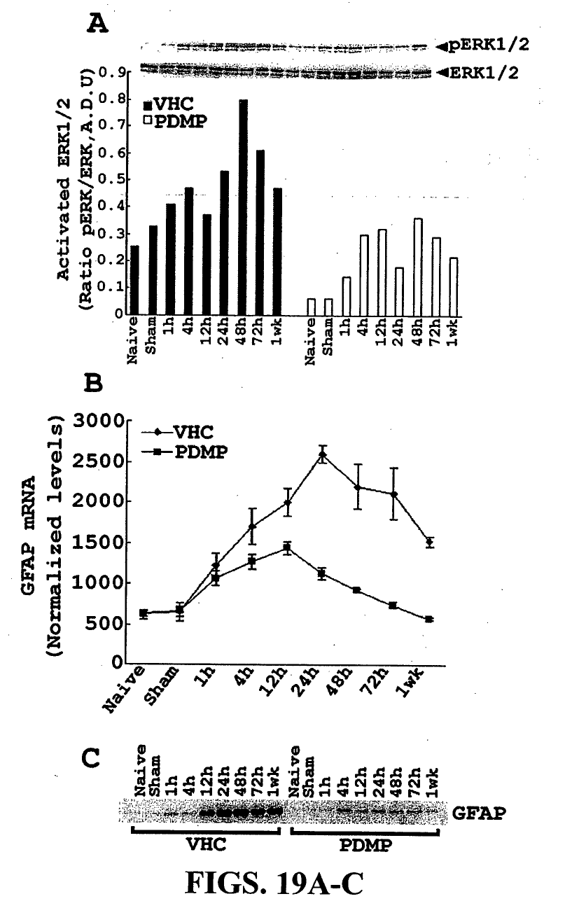

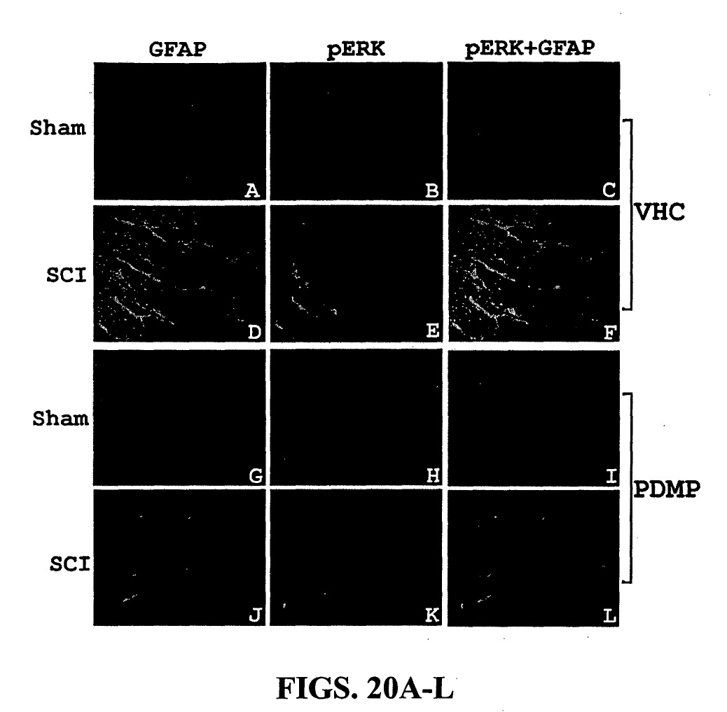

To test the physiological relevance of the above observations and further investigate the role of LacCer in astrogliosis in vivo, the inventors examined the effect of PDMP in the rat SCI model. Rapid and chronic activation of ERK1/2 has been proposed to be a mechanism that operates in astroglial activation following acute brain injury (Mandell and VandenBerg, 1999; Mandell et al., 2001). Furthermore, astrogliosis triggered in response to secondary inflammatory disease has been widely reported to be detrimental for axonal regeneration and recovery in SCI (Fawcett and Asher, 1999; Rabchevsky and Smith, 2001; Profyris et al., 2004). As shown in FIG. 7A, a robust activation of ERK1/2 is observed within 1 h post-SCI. Activated ERK1/2 levels steadily rise until 48 h post-SCI and remain substantially elevated even 1 wk post-SCI. However, PDMP (20 mg/kg) treatment post-SCI effectively attenuates chronic ERK1/2 activation (FIG. 19A). Additionally, PDMP treatment effectively attenuated GFAP mRNA (FIG. 19B) and protein expression (FIG. 19C) which was highly up-regulated in VHC-treated SCI. Furthermore double immunofluorescence analysis of spinal cord sections from the lesion epicenter of vehicle (VHC)-treated SCI rats showed a significant increase in GFAP (FIG. 20D) and activated ERK1/2 (FIG. 20E) levels and their co-localization (FIG. 20F) 24 h following injury, whereas PDMP-treated SCI rats showed significantly attenuated GFAP (FIG. 20J) activated ERK1/2 (FIG. 20K) and their co-localization (FIG. 20L), thus demonstrating the efficacy of PDMP in vivo in controlling chronic ERK1/2 activation and GFAP expression resulting in the attenuation of post-SCI astrogliosis. Thus these studies indicate the involvement of glycosphingolipids in astrogliosis at the site of lesion in an in vivo model of SCI. These observations find critical relevance in other neuroinflammatory diseases as well since astrogliosis and its detrimental effects are common to a number of CNS disorders.

### Discussion

Astrogliosis is a prominent and ubiquitous reaction of astrocytes characterized by proliferation of astrocytes with up-regulated expression of GFAP (Hatten et al., 1991; Eddleston and Mucke, 1993; Neary et al., 1994; Norenberg, 1994; Ridet et al., 1997; Profyris et al., 2004). Although the functional role of astrogliosis is not clearly defined, numerous studies have documented its pathological interference with the function of residing neuronal circuits, thus, preventing axonal remyelination and inhibiting axonal regeneration (Eng et al., 1992; Houle and Tessler, 2003). The inventors have previously reported the involvement of LacCer in inducible nitric oxide synthase gene expression in primary astrocytes and the anti-inflammatory efficacy of PDMP-treatment in protecting against white matter vacuolization, demyelination and neuronal apoptosis resulting in profoundly improved neurological outcome in a rat model of SCI (Pannu et al. 2004, in press). Since PDMP treatment profoundly attenuated the inflammatory disease process post-SCI including GFAP expression which is a characteristic feature of astrogliosis, in this example the inventors sought to investigate the involvement of GSL in proliferation of astrocytes and GFAP expression, the two processes that culminate in astrogliosis. This example demonstrates a novel pathway of S1P and LacCer-mediated regulation of TNFα-induced astrocyte proliferation and GFAP expression through signaling events involving PI-3K and the Ras/ERK1/2 pathway in primary astrocytes. These conclusions are based on the following findings. (1) TNFα-stimulation induced the activity of GalT-2 and increased the production of LacCer. (2) The inhibition of GSL synthesis by PDMP or antisense oligoucleotides to GalT-2 inhibited astrocytes proliferation and GFAP expression which was reversed by LacCer but not other GSLs (GluCer, GalCer, GM1, GM3 and GD3). (3) Inhibition of LacCer synthesis also inhibited the activation of Ras/ERK1/2 pathway. (4) TNFα-induced cellular proliferation and LacCer generation was found to be regulated by SIP through activation of PI-3K. (5) PI-3K through an as yet unknown mechanism regulated GalT-2 enzyme activity and LacCer production. (6) PDMP treatment effectively attenuated chronic ERK1/2 activation and GFAP expression in a rat model of SCI. FIG. 12 shows a schematic representation of the possible regulation of TNFα-induced astrocyte proliferation and GFAP expression by S1P and LacCer. TNFα, a pro-inflammatory cytokine is a well documented agonist of sphingosine kinase inducing rapid generation of S1P (Maceyka et al., 2002; Vann et al., 2002; Pettus et al., 2003). TNFα-generated SIP activates PI-3K, a major pro-survival and mitogenic pathway (Neri et al., 2002; Takeda et al., 2004) which results in the activation of GalT-2 resulting in LacCer biosynthesis. LacCer generation recruits and activates the small GTPase Ras that activates the downstream ERK1/2 pathway thus resulting in astrocyte proliferation and GFAP expression and triggering astrogliosis. Reports from our laboratory and others have reported the mechanism for the LacCer-mediated regulation of Ras to be dependent reactive oxygen species dependent in primary astrocytes (Pannu et al., 2004; in press) and other cell types (Bhunia et al., 1997). TNFα-induced activation of the small GTPase Ras could be through the direct activation of Src kinases associated with the LacCer-enriched glycosphingolipid signaling domains (GSD) present on the cell surface. A number of studies have shown that several transducer molecules such as Src kinase, associate with these GSD and form functional units which mediate signal transduction and cellular functions (Brown and London, 1998). In particular, a Src kinase, Lyn, has been found to directly associate with LacCer resulting in superoxide generation via NADPH oxidase activation in neutrophils (Iwabuchi and Nagaoka, 2002). Src kinase activation possibly leading to ROS generation may be followed by Grb/SOS-mediated Ras activation that triggers the downstream, MEK1/2-ERK1/2 pathway. The data presented in this example identify a glycosphingolipid, LacCer, as a bioactive signaling molecule regulating astrogliosis by mediating astrocyte proliferation and GFAP expression. In addition, the blockade of trauma-mediated ERK activation and GFAP expression in SCI model (as reported in this example) and the inflammatory process and neuronal apoptosis (as reported earlier) by PDMP further establishes LacCer, generated through GalT-2 stimulation, to be a potent signaling lipid molecule that triggers inflammation and astrogliosis in various neuroinflammatory diseases.

Glial cells can secrete TNFα, which, in turn, can act on these cells in an autocrine manner. TNFα can induce the proliferation of astrocytes (Barna et al., 1990; Selmaj et al., 1990) and overexpression of GFAP (Zhang et al., 2000), a process known as astrogliosis. Astrogliosis is a prominent and ubiquitous reaction of astrocytes to many forms of CNS injury, often implicated in the poor regenerative capacity of the adult mammalian CNS (Tatagiba et al., 1997). As in any other CNS injury, SCI initiates reactive gliosis as part of a response to restore homeostasis at the site of primary injury. However, with this comes the unfortunate burden of massive deposition of molecules that inhibit axonal growth and recovery (Fawcett and Asher, 1999). TNFα, a potent pleiotropic pro-inflammatory cytokine is generated during the inflammatory response in SCI. Within 15 mins the mRNA levels of TNFα are increased in most cellular components of the CNS (Arvin et al., 1996; Bartholdi and Schwab, 1997; Klusman and Schwab, 1997; Yan et al., 2001). Although the levels of other pro-inflammatory cytokines are barely detectable after 24 h the protein levels of TNFα continue to increase during the first week following SCI (Tyor et al., 2002) probably attributable to leukocyte infiltration and secretion of pro-inflammatory cytokines at the site of primary injury (Popovich and Jones, 2003; Popovich et al., 2003). Although the functional role of astrogliosis is not clearly defined, numerous studies have documented its pathological interference with the function of residing neuronal circuits, thus, preventing axonal remyelination and inhibiting axonal regeneration (Steeves and Tetzlaff, 1998). A number of strategies have been tested for modulation of astrogliosis following neurotrauma such as ablation of astrocytes (Yajima and Suzuki, 1979; Moon et al., 2000), alteration of the extracellular matrix (ECM) associated with the astroglial scar (Fichard et al., 1991) but with mixed results (McGraw et al., 2001).

The inventors have previously demonstrated the anti-inflammatory potential of PDMP, a glycosphingolipid synthesis inhibitor, for treating SCI-induced inflammatory disease in a rat model of SCI (Pannu et al.; 2004 in press). PDMP treatment post-SCI until 72 h after injury showed a profoundly improved neurological outcome post-SCI as compared to the untreated rats. The mechanism of protection was found to be through attenuation of astrocytes derived inducible nitric oxide synthase gene expression. Through in vitro studies, regulation of iNOS expression in primary astrocytes was found to be mediated by LacCer, a GSL derivative through the Ras/ERK/NF-kB pathway (Pannu et al. 2004 in press). The ERK pathway has been reported to be chronically activated in human reactive astrocytes in subacute and chronic lesions including infarct, mechanical damage, chronic epilepsy and progressive multifocal meukocephalopathy (Mandell et al., 2001; Yanase et al., 2001). Since neurons, oligodendrocytes and most inflammatory cells showed little or not detectable activation, the activation for the ERK pathway has been deemed obligatory for the triggering and persistence of reactive astrocytes (Mandell and VandenBerg, 1999). Furthermore, this example established ERK activation observed in astrogliosis to be SIP- and LacCer-mediated. Activated ERK co-localized with GFAP over expressing reactive astrocytes in spinal cord sections post-SCI (FIG. 8F). The fact that the chronic activation of ERK following SCI was markedly attenuated by PDMP treatment (FIG. 7A). it further established LacCer as a bioactive signaling lipid involved not only in iNOS gene expression but also capable of inducing astrogliosis in SCI.

Since the discovery of the sphingomyelin (SM) cycle, which involves sphingomyelin hydrolysis by sphingomyelinases (SMases) resulting in ceramide generation, several inducers including TNFα have been shown to be coupled to sphingomyelin-ceramide signaling events (Hannun, 1994; Kolesnick et al., 1994; Kanety et al., 1995; Linardic et al., 1996). Instead of viewing enzymes of sphingolipid metabolism as isolated signaling modules, these pathways are now accepted to be highly interconnected with the product of one enzyme serving as a substrate for the other. This is also true of ceramide generated through the SM cycle or de novo as ceramide can be converted into other bioactive molecules such as sphingosine, sphingosine-1-phosphate or glycosphingolipids. The complexity of these bioactive sphingolipids is accentuated by growing evidence of the presence of ceramide and other derivatives such as LacCer and gangliosides in lipid-enriched microdomains within membranes. These microdomains, called ‘lipid rafts’, have a number of receptors, including those for TNFα, and signaling molecules clustered within or associated with them thus making them critical for signaling events which mediated numerous cellular processes and at the same time are imperative for cellular proliferation as reported for oligodendrocytes (Hakomori and Handa, 2003; Decker and ffrench-Constant, 2004).

The metabolic interconnections of ceramide and other lipids mediators such as sphingosine, sphingosine-1-phosphate (S-1-P) and glycosphingolipids make predicting the specific actions of these intermediates and the enzymes regulating their levels rather complex. For example, while sphingosine has pro-apoptotic effects like ceramide depending on cell type (Spiegel and Merrill, 1996) its rapid conversion to S-1-P has proliferative properties antagonistic to those of sphingosine and ceramide and has been implicated in proliferation of various cell types including primary cortical astrocytes (Spiegel and Milstien, 2000; Osawa et al., 2001; Yamagata et al., 2003). In pathological situations resulting from brain trauma and haemorrhage, platelets and infiltrating immune cells have been shown to be a source of S1P which contact the nervous cells (Pebay et al., 2001). TNFα has been shown to potently induce SIP generation through a sphingosine kinase dependent manner (Pettus et al., 2003). In this example a complex interconnection between the SIP pathway and GSL pathway was established whereby LacCer production was found to be SIP-dependent. SIP was found to mediate TNFα-induced astrocyte proliferation by activation of the PI-3K which in turn was responsible for GalT-2 activation resulting in LacCer generation and astrogliosis through the ERK pathway. So far substantial attention has been focused on trying to map the interconversion of the various metabolites of the sphingolipid pathway which has a profound effect on the outcome of a certain stimuli. However, the regulation of LacCer synthesis by SIP brings attention to the fact that these bioactive molecules are not simply being interconverted but are involved in synthesis of other sphingolipid derivatives through signaling events triggered by them.

In conclusion the inventors have step-by-step dissected signaling pathway involved in TNFα-induced astrocyte proliferation and GFAP expression and established the connection between two major mitogenic lipids, SIP and LacCer in mediating these processes. The results presented further establish LacCer as a significant bioactive lipid molecular capable of mediating inflammatory disease process in SCI as opposed to earlier perception of LacCer as simply a precursor for complex gangliosides. At present the ongoing challenge for research focused on spinal cord regeneration is to modulate astrocytes' response to injury so as to gain from its potential neurotrophic effects while at the same time tempering its scarring effect. This report proposes GSL modulation as a potential tool to attenuate astrogliosis and the inflammatory disease processes in neuroinflammatory diseases.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Ser. No. 10/273,557
U.S. Ser. No. 10/429,231
U.S. Pat. No. 5,041,441
U.S. Pat. No. 5,696,109
U.S. Pat. No. 5,707,649
U.S. Pat. No. 5,707,649
U.S. Pat. No. 6,117,447
U.S. Pat. No. 6,511,800
U.S. Pat. No. 6,511,800
U.S. Pat. No. 6,569,889
U.S. Prov. 60/066,839
U.S. Prov. 60/531,828
U.S. Prov. 60/559,112
U.S. Appln. 2001/0041735
U.S. Appln. 2002/0198240
U.S. Appln. 2003/0069200
U.S. Appln. 2003/0073680
Abe et al., Curr. Drug Metab., 2(3):331-338, 2001.
Akiyama et al., Alzheimer Dis. Assoc. Disord., 14(1):S47-53. 2000.
Alter, Ann. NY Acad. Sci., 845:391-401, 1998.
Andersson et al., J. Exp. Med., 176:255-259, 1992.
Ando et al., Jpn. J. Exp. Med., 54:229-234, 1984.
Arvin et al., Neurosci. Biobehav. Rev., 20:445-452, 1996.
Bagasra et al., Proc. Natl. Acad. Sci. USA, 92:12041-12045, 1995.
Bal-Price and Brown, J. Neurosci., 21:6480-6491, 2001.
Banno et al., J. Biol. Chem., 276:35622-35628, 2001.
Bareyre and Schwab, Trends Neurosci., 26:555-563, 2003.
Barna et al., J. Neuroimmunol., 30:239-243, 1990.
Bartholdi and Schwab, Eur. J. Neurosci., 9:1422-1438, 1997.
Basso et al., Exp. Neurol., 139:244-256, 1996.
Beasley and Brenner, Kidney Int., 42(38):S96-S100, 1992.
Beckman et al. Proc. Natl. Acad. Sci. USA, 87:1620-1624, 1990.
Berisha et al., Proc. Natl. Acad. Sci. USA, 91:744-749, 1994.
Bethea et al., J. Neuroimmunol., 30:1-13, 1990.
Bhunia et al., J. Biol. Chem., 271:10660-10666, 1996.
Bhunia et al., J. Biol. Chem., 272:15642-15649, 1997.
Bhunia et al., J. Biol. Chem., 273:34349-34357, 1998.
Blight Neuroscience. 10: 1471-1486, 1983.
Bo et al., Ann. Neurol., 36:778-786, 1994.
Bonfoco et al., Proc. Natl. Acad. Sci. USA, 92:7162-7166, 1995.
Boughton-Smith et al., Lancet., 342:338-340, 1993.
Bredesen, Ann. Neurol., 38:839-851, 1995.
Breitner et al., Neurology, 44(2):227-232, 1994.
Brown and London, Annu. Rev. Cell Dev. Biol., 14:111-136, 1998.
Brugg et al., J. Neurochem., 66:733-739, 1996.
Buisson et al., Br. J. Pharmacol., 106:766-767, 1992.
Bull et al., J. Invest. Derm., 103:435, 1994.
Busse et al., FEBS Lett., 275:87-90, 1990.
Cartier et al., Proc. Natl. Acad. Sci. USA, 92:1674-1678, 1995.
Castro-Palomino et al., Chemistry, 7:2178-2184, 2001.
Chatterjee et al., Glycobiology, 7:703-710, 1997.
Chatterjee, Arterioscler. Thromb. Vasc. Biol., 18:1523-1533, 1998.
Chen and Rosazza, Biochem. Biophys. Res. Commun., 203:1251-1258, 1994.
Chiueh et al., Free Radic. Biol. Med., 13(5):581-583, 1992.
Corbett and McDaniel, Methods Enzymol., 268:398-408, 1996.
Cross et al., J. Clin. Invest., 93:2684-2690, 1994.
Cuvillier et al., J. Biol. Chem., 275:15691-15700, 2000.
Davaille et al., J. Biol. Chem., 277:37323-37330, 2002.
Dawson et al., NIDA Res. Monogr., 136:258-271, 1993.
Dawson et al., Proc. Natl. Acad. Sci. USA, 88:6368-6371, 1991.
Decker and French-Constant, J. Neurosci., 24:3816-3825, 2004.
Devlin et al., Transplantation, 58:592-595, 1994.
Dignam et al., Methods Enzymol., 101:582-598, 1983.
Dihne et al., Brain Res., 902:178-189, 2001.
Drapier et al., J. Immunol., 140:2829-2838, 1988.
Eisieik and Leijersfam, Diabetes Metabolism, 20:116-122, 1994.
Eng et al., Prog. Brain Res., 94:353-365, 1992.
Evans et al., Infec. Imm., 60:4133-4139, 1992.
Fawcett and Asher, Brain Res. Bull., 49:377-391, 1999.
Fenyk-Melody et al., J. Immunol., 160:2940-2946, 1998.
Fern, Prog. Brain Res., 132:405-411, 2001.
Fichard et al., Dev. Biol., 148:1-9, 1991.
Galea et al., Proc. Natl. Acad. Sci. USA, 89:10945-10949, 1992.
Geisler, Ann. NY Acad. Sci., 845: 374-381, 1998.
Geng et al., J. Cell. Physiol., 163:545-554, 1995.
Gilg et al., J. Neuropathol. Exp. Neurol., 59:1063-1069, 2000.
Giri et al., Faseb J., 16:661-672, 2002.
Goureau et al., Biochem. Biophys. Res. Commun., 186:854-859, 1992.
Green et al., Anal. Biochem., 126:131-138, 1982.
Gruner, J. Neurotrauma., 9:123-126, 1992.
Haga et al., Brain Res., 601:88-94, 1993.
Hakomori and Handa, Methods Enzymol., 363:191-207, 2003.
Hamid et al., Lancet., 342:1510-1513, 1993.
Hannun, J. Biol. Chem., 269:3125-3128, 1994.
Hardy et al., J. Immunol., 153:1754-1761, 1994.
Hashmi et al., FEBS Lett., 196:247-250, 1986.
Hatten et al., Glia, 4:233-243, 1991.
Herrmann et al., J. Biol. Chem., 270:2901-2905, 1995.
Hooper et al., Proc. Natl. Acad. Sci. USA, 95:675-680, 1998.
Hooper et al., Proc. Natl. Acad. Sci. USA., 94:2528-2533, 1997.
Houle and Tessler, Exp. Neurol., 182:247-260, 2003.
Hu et al., Glia, 15:491-494, 1995.
Ialenti et al., Br. J. Pharmacol., 110:701-706, 1993.
Inokuchi and Radin, J. Lipid Res., 28:565-571, 1987.
Inokuchi et al., Acta Biochim. Pol., 45:479-492, 1998.
Inokuchi et al., J. Cell. Physiol., 141:573-583, 1989.
Issazadeh et al., J. Neurosci. Res., 40:579-590, 1995.
Iwabuchi and Nagaoka, Annu. Rev. Cell Dev. Biol., 11:417-440, 1995.
Joshi et al., Res. Commun. Mol. Pathol. Pharmacol. Mar., 91:339-346, 1996.
Kanety et al., J. Biol. Chem., 270:23780-23784, 1995.
Kaurs and Halliwell, FEBS Lett., 350:9-12, 1994.
Kawai et al., Neurosci. Lett., 174:225-227, 1994.
Kernie et al., J. Neurosci. Res., 66:317-326, 2001.
Khan et al., J. Neurochem., 71:78-87, 1998.
Kharitonov et al., Lancet., 343:133-135, 1994.
Kiernan, Trends Pharmacol. Sci., 11:316, 1990.
Kilbourn et al., Proc. Natl. Acad. Sci. USA, 87:3629-3632, 1990.
Klinkert et al., J. Neuroimmunol., 72(2)163-168, 1997.
Klusman and Schwab, Brain Res., 762:173-184, 1997.
Kolb-Bachofen et al., Lancet., 344:139 1994.
Kolesnick et al., Biochem. Cell Biol., 72:471-474, 1994.
Koprowski et al., Proc. Natl. Acad. Sci. USA, 90:3024-3027, 1993.
Kracun et al., Periodicum Biologorum, 97(2):113-118, 1995.
Kroncke et al., Biochem. Biophys. Res. Commun., 175:752-758, 1991.
Kubes et al., Proc. Natl. Acad. Sci. USA, 88:4651-4655, 1991.
Kubota et al., Acta Neurochir. Suppl., 76:339-341, 2000.
Lachman et al., J. Immunol., 138:2913-2916, 1987.
Lander et al., J. Biol. Chem., 270:21195-21198, 1995.
Lassmann and Wisniewski, Arch. Neurol., 36:490-497, 1979.
Lazo et al., Proc. Natl. Acad. Sci. USA, 85:7647-7651, 1988.
Lee et al., NeuroReport, 3:841-844, 1992.
Leist et al., Eur. J. Neurosci., 9:1488-1498, 1997.
Li et al., Cancer Res., 56:4602-4605, 1996.
Linardic et al., Cell Growth Differ., 7:765-774, 1996.
Liu et al., J. Biol. Chem., 274:1140-1146, 1999.
Liu et al., J. Neurosci., 17:5395-5406, 1997.
Lowick et al., J. Clin. Invest., 93:1465-1472, 1994.
Maceyka et al., Biochim. Biophys. Acta, 1585:193-201, 2002.
Maimone et al., J. Neurolmmunol., 32:67-74, 1991.
Mandell and VandenBerg, Neuroreport, 10:3567-3572, 1999.
Mandell et al., Glia, 34:283-295, 2001.
Mandia et al., Invest Opthalmol., 35:3673-3689, 1994.
Marcus et al., Glia, 41:152-160, 2003.
Marietta, Trends Biochem. Sci., 14:488, 1989.
Matsuyama et al., J. Spinal Disord., 11:248-252, 1998.
McCartney-Francis et al., J. Exp. Med., 178:749-754, 1993.
McGraw et al., J. Neurosci. Res., 63:109-115, 2001.
McGuiness et al., J. Neuroimmunol., 75:174-182, 1997.
McGuinness et al., J. Neuroimmunol., 61:161-169, 1995.
Meller et al., Europ. J. Pharmacol., 214:93-96, 1992.
Mendez-Otero and Cavalcante, Prog. Mol. Subcell. Biol., 2:97-124, 2003.
Merrill and Benveniste, Trends Neurosci., 19:331-338, 1996.
Merrill et al., J. Immunol., 151:2132-2141, 1993.
Miller et al., Br. J. Surg., 77(8):895-897, 1990.
Miller et al., J. Pharmacol. Exp. Ther., 264:11-16, 1993.
Miller et al., Lancet., 34:465-466, 1993.
Mitrovic et al., Neurosci., 61:575-585, 1994.
Mizutani et al., Biochem. Biophys. Res. Commun., 222(2):494-498, 1996.
Moncada et al. Biochem. Pharmacol., 38:1709-1715, 1989.
Moon et al., Exp. Neurol., 161:49-66, 2000.
Moore et al., Brit. J. Pharmacol., 108:296-297, 1992.
Moore et al., Brit. J. Pharmacol., 102:198-202, 1991.
Moser et al., Ann. Neurol., 16:628-641, 1984.
Moser, J. Neuropathol. Expt. Neurol., 54:740-744, 1995.
Mosser et al., Nature, 361:726-730, 1993.
Mucke and Eddleston, Faseb J, 7:1226-1232, 1993.
Muhl et al., Br. J. Pharmcol., 112:1-8, 1994.
Mulligan et al., Br. J. Pharmacol., 107:1159-1162, 1992.
Nagafuji et al., Neurosci., 147:159-162, 1992.
Nathan, FASEB J, 6:3051-3064, 1992.
Nathan, J. Clin. Invest., 79:319-326, 1987.
Neary et al., J. Neurochem., 63:2021-2027, 1994.
Neri et al., Biochim. Biophys. Acta, 1584:73-80, 2002.
Norenberg J. Neuropathol. Exp. Neurol., 53:213-220, 1994.
Ohtani et al., Neurodegeneration, 5:331-338, 1996.
Olesen et al., Trends Pharmacol. Sci., 15:149-153, 1994.
Osawa et al., J. Immunol., 167:173-180, 2001.
Pahan et al., Free Radic. Biol. Med., 24:39-48, 1998a.
Pahan et al., J. Biol. Chem., 273:2591-2600, 1998b.
Pahan et al., J. Biol. Chem., 274:7528-7536, 1999.
Pahan et al., J. Neurochem., 74:2288-2295, 2000.
PCT Appln. WO 84/03564
PCT Appln. WO 99/26657
Pebay et al., Eur. J. Neurosci., 13:2067-2076, 2001.
Perry et al., Neurobiol. Aging, 22:873-883, 2001.
Petros et al., Lancet., 338:1557-1558, 1991.
Pettus et al., Faseb J, 17:1411-1421, 2003.
Popovich and Jones, Trends Pharmacol. Sci., 24:13-17, 2003.
Popovich et al., Exp. Neurol., 182:275-287, 2003.
Powers et al., J. Neuropathol. Expt. Neurol., 51:630-643, 1992.
Powers, J. Neuropathol. Expt. Neurol., 54:710-719, 1995.
Profyris et al., Neurobiol. Dis., 15:415-436. 2004.
Rabchevsky and Smith, Arch. Neurol., 58:721-726, 2001.
Radi et al., J. Biol. Chem., 266:4244-4250, 1991.
Remington's Pharmaceutical Sciences, 18th Ed. Mack Printing Company, 1289-1329, 1990.
Renno et al., J. Immunol., 154:944-953, 1995.
Ridet et al., Trends Neurosci., 20:570-577, 1997.
Rizzo et al., Neurology, 36:357-361, 1986.
Rizzo et al., Neurology, 39:1415-1422, 1989.
Rogers et al., Neurology, 43(8):1609-1611, 1993.
Rogers et al., Proc. Natl. Acad. Sci. USA, 89(21):10016-10020, 1992.
Rosner, Prog. Mol. Subcell. Biol., 32:49-73, 2003.
Rudick and Ransohoff, Arch. Neurol., 49:265-270, 1992.
Ruuls et al., Clin. Exp. Immunol., 103:467-474, 1996.
Ruzicka et al., J. Invest. Derm., 103:397, 1994.
Saklatvala et al., Philos. Trans. R Soc. Lond. B. Biol. Sci., 351:151-157, 1996.
Saran et al., Free Rad. Res. Commun., 10:221-226, 1990.
Satake et al., Brain Res. Mol. Brain. Res., 85:114-122, 2000.
Schilling et al., Intensive Care Med., 19:227-231, 1993.
Schneider, Ann. NY Acad. Sci., 845:363-373, 1998.
Selmaj et al., J. Immunol., 144:129-135, 1990.
Sequeira et al., Nitric Oxide, 1:315-329, 1997.
Shafer and Murphy, Glia, 21:370-379, 1997.
Simmons and Murphy, J. Neurochem., 59:897-905, 1992.
Simon et al., Cell Death Differ., 9:758-767, 2002.
Singh et al., J. Biol. Chem., 273:20354-20362, 1998.
Singh, Mol. Cell. Biochem., 167:1-29, 1997.
Spiegel and Merrill Jr., Faseb J, 10:1388-1397, 1996.
Spiegel and Milstien, FEBS Lett., 476:55-57, 2000.
Stadler et al., J. Immunol., 147:3915-3920, 1991.
Steeves and Tetzlaff, Ann. NY Acad. Sci., 860:412-424.
Stevanovic-Racic et al., Arth. Rheum., 37, 1062-1069, 1994.
Suzuki et al., Naunyn Schmiedebergs Arch. Pharmacol., 363:94-100, 2001.
Takeda et al., Pancreas, 28:353-358, 2004.
Tanti et al., J. Biol. Chem., 271:25227-25232, 1996.
Tatagiba et al., Neurosurgery, J40:541-546, 1997.
Taupin et al., Eur. J. Immunol., 27:905-913, 1997.
Thiemermann and Vane, Eur. J. Pharmacol., 211:172-182, 1992.
Thompson, Science, 267:1456-1462, 1995.
Trifiletti et al., Europ. J. Pharmacol., 218:197-198, 1992.
Tsukada et al., J. Neurol. Sci., 104:230-234, 1991.
Tyor et al., Neurol. Sci., 200:33-41, 2002.
Uemura et al., J. Biochem (Tokyo), 110(1):96-103, 1991.
Usuki et al., J. Neurochem., 67(5):1821-1830, 1996.
van der Veen et al., J. Neuroimmunol., 77:1-7, 1997.
Vann et al., J. Biol. Chem., 277:12649-12656, 2002.
Villarroya et al., J. Neuroimmunol., 64:55-61, 1996.
Vodovotz et al., J. Exp. Med., 184:1425-1433, 1996.
Wada et al., J. Neurosurg., 89:807-818, 1998a.
Wada et al., Neurosurgery, 43:1427-1436, 1998b.
Ward et al., J. Biol. Chem., 267:23862-23869 1992.
Weber et al., Br. J. Pharmacol., 123(5):906-910, 1998.
Welsh et al., Endocrinol., 129:3167-3173, 1991.
White et al., Proc. Natl. Acad. Sci. USA, 91:1044-1048, 1994.
Wiesner and Dawson, J. Neurochem., 66:1418-1425, 1996.
Wink et al., Science, 254:1001-1003, 1991.
Winlaw et al., Lancet., 344:373-374, 1994.
Won et al., Brain Res., 903:207-215, 2001.
Won et al., J. Neurochem., 88:583-593, 2004.
Wrathall et al., Exp. Neurol., 137:119-126, 1996.
Wrathall, J. Neurotrauma., 9(1) 1: S129-134, 1992.
Xie et al., J. Biol. Chem., 269:4705-4708, 1994.
Yajima and Suzuki, Lab. Invest., 41:385-392, 1979.
Yamagata et al., Glia, 41:199-206, 2003.
Yamagishi et al., Eur. J. Pharmacol., 462(1-3):53-60, 2003.
Yan et al., J. Neurotrauma., 18:563-568, 2001.
Yanase et al., Pigment Cell Res., 14:103-109, 2001.
Yeh et al., J. Vasc. Res., 38:551-559, 2001.
Young, J. Emerg. Med, 11(1):13-22, 1993.
Yu et al., J. Neurochem., 23:169-174, 1974.
Zang et al., J. Exp. Med., 177:511-516, 1993.
Zembowicz and Vane, Proc. Natl. Acad. Sci. USA, 89:2051-2055, 1992.
Zhang et al., Neuroreport., 11:409-412, 2000.
Zielasek et al., Cell Immunol., 141:111-120, 1992.

1. A method of treating type I diabetes in a subject, comprising administering a biologically effective amount of a glycosphingolipid inhibitor to the subject in need of such treatment.

2. The method of claim 1, wherein the glycosphingolipid inhibitor is an inhibitor of glucosylceramide synthase.

3. The method of claim 1, wherein the glycosphingolipid inhibitor is an inhibitor of GalT-2.

4. The method of claim 1, wherein the subject is a mammal.

5. The method of claim 4, wherein the mammal is a human.

6. The method of claim 4, wherein the biologically effective amount is administered to said mammal.

7.-14. (canceled)

15. The method of claim 1, wherein the glycosphingolipid inhibitor is a PDMP derivative.

16. The method of claim 15, wherein the PDMP derivative is D-threo-3′,4′-ethylenedioxy-1-phenyl-2-palmitoylamino-3-pyrrolidino-1-propanol or D-threo-4′-hydroxy-1-phenyl-2-palmitoylamino-3-pyrrolidino-1-propanol.

17. The method of claim 1, wherein the glycosphingolipid inhibitor is N-butyldeoxynojirimycin.

18. The method of claim 1, wherein the glycosphingolipid inhibitor is Miglustat.

19. The method of claim 1, wherein the glycosphingolipid inhibitor is an inhibitor of sphingosine kinase or sphingosine-1-phosphate phosphatase.

20. The method of claim 1, wherein the glycosphingolipid inhibitor is PDMP.

21. The method of claim 20, wherein the PDMP is in a pharmaceutically acceptable excipient.

22. The method of claim 21, wherein the PDMP is administered with a second pharmaceutical preparation.

23. The method of claim 22, wherein the second pharmaceutical preparation enhances intracellular cAMP.

24. The method of claim 23, wherein the second pharmaceutical preparation is Rolipram.

25. The method of claim 22, wherein the second pharmaceutical preparation comprises GM1.

26. The method of claim 22, wherein the second pharmaceutical preparation comprises an inhibitor of mevalonate synthesis, an inhibitor of the farnesylation of Ras, an antioxidant, an enhancer of intracellular cAMP, an enhancer of protein kinase A (PKA), an inhibitor of NF-κ.beta. activation, an inhibitor of Ras/Raf/MAP kinase pathway, an inhibitor of mevalonate pyrophosphate decarboxylase or an inhibitor of farnesyl pyrophosphate.

27.-43. (canceled)
